SEQUENCE LISTING (52 sequences)

SEQ ID NO: 1 - DNA, 4751 bp, Homo sapiens
  source 1..4751: mol_type = genomic DNA
  misc_feature 1..50: note = flanking sequence
  exon 51..323
  intron 324..2182
  exon 2183..2227
  intron 2228..3101
  exon 3102..3209
  intron 3210..4148
  exon 4149..4701
  3'UTR 4149..4701
  misc_feature 4702..4751: note = flanking sequence
  misc_feature 1..4751: note = TRAC gene sequence, ENST00000611116, taken from ensembl.org
caaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttcattaaaatgatttggaagagcagagactgtgcctctgtttgactgggtttggtaggagtcattttctgcttgct

SEQ ID NO: 2 - RNA, 20 nt, synthetic construct
  source 1..20: mol_type = other RNA
  misc_feature 1..20: note = Nucleic acid guide #1 (against TRAC)
ttcggaacccaatcactgac

SEQ ID NO: 3 - RNA, 20 nt, synthetic construct
  source 1..20: mol_type = other RNA
  misc_feature 1..20: note = Nucleic acid guide #2 (against TRAC)
tgtggtccagctgaggtgag

SEQ ID NO: 4 - RNA, 79 nt, synthetic construct
  source 1..79: mol_type = other RNA
  misc_feature 1..79: note = Guide RNA scaffold
ggaaccattcaaaacagcatagcaagttaaaataaggctagtccgttatcaacttgaaaaagtggcaccgagtcggtgc

SEQ ID NO: 5 - DNA, 20 bp, synthetic construct
  source 1..20: mol_type = other DNA
  misc_feature 1..20: note = TRAC Forward Primer
tgagcacctaccccatcccc

SEQ ID NO: 6 - DNA, 21 bp, synthetic construct
  source 1..21: mol_type = other DNA
  misc_feature 1..21: note = TRAC Reverse Primer
agtaggagagtttggtgggct

SEQ ID NO: 7 - DNA, 20 bp, synthetic construct
  source 1..20: mol_type = other DNA
  misc_feature 1..20: note = TCRbeta1 Forward Primer
accctggctccaacccctct

SEQ ID NO: 8 - DNA, 21 bp, synthetic construct
  source 1..21: mol_type = other DNA
  misc_feature 1..21: note = TCRbeta1 Reverse Primer
tcatggtgtgcgctggttcct

SEQ ID NO: 9 - DNA, 21 bp, synthetic construct
  source 1..21: mol_type = other DNA
  misc_feature 1..21: note = PD1 Forward Primer
ttgctgcccgagggatgtgag

SEQ ID NO: 10 - DNA, 22 bp, synthetic construct
  source 1..22: mol_type = other DNA
  misc_feature 1..22: note = PD1 Reverse Primer
ctgatcctgtgcaggaggggac

SEQ ID NO: 11 - DNA, 19 bp, synthetic construct
  source 1..19: mol_type = other DNA
  misc_feature 1..19: note = Forward primer (expression vector site)
tcaggtgtcgtgaggatcc

SEQ ID NO: 12 - DNA, 22 bp, synthetic construct
  source 1..22: mol_type = other DNA
  misc_feature 1..22: note = Reverse primer (expression vector site)
cataaggtcatgtactgggcac

SEQ ID NO: 13 - DNA, 20 bp, synthetic construct
  source 1..20: mol_type = other DNA
  misc_feature 1..20: note = Target sequence of nucleic acid guide #1 (in TRAC)
gtcagtgattgggttccgaa

SEQ ID NO: 14 - DNA, 20 bp, synthetic construct
  source 1..20: mol_type = other DNA
  misc_feature 1..20: note = Target sequence of nucleic acid guide #2 (in TRAC)
ctcacctcagctggaccaca

SEQ ID NO: 15 - DNA, 20 bp, synthetic construct
  source 1..20: mol_type = other DNA
  misc_feature 1..20: note = Target sequence of nucleic acid guide #3 (in PD1)
cacagaggtaggtgccgctg

SEQ ID NO: 16 - DNA, 9707 bp, synthetic construct
  source 1..9707: mol_type = other DNA
  regulatory 3..229: note = Rous sarcoma virus enhancer/promoter; regulatory_class = promoter
  misc_feature 230..410: note = truncated 5' long terminal repeat (LTR) from HIV-1
  misc_feature 457..582: note = HIV-1 Psi; note = packaging signal of human immunodeficiency virus type 1
  misc_feature 1075..1308: note = RRE; note = The Rev response element (RRE) of HIV-1 allows for Rev- dependent mRNA export from the nucleus to the cytoplasm
  misc_feature 1803..1920: note = central polypurine tract (cPPT) and central termination sequence (CTS) of HIV-1
  regulatory 1965..2930: note = strong constitutive promoter for human elongation factor EF-1-alpha; regulatory_class = promoter
  intron 2196..2930: note = intron upstream of the start codon of human EF-1-alpha
  intron 2931..3134: note = truncated intron upstream of the start codon of human EF- 1-alpha
  regulatory 3157..3162: note = vertebrate consensus sequence for strong initiation of translation (Kozak, 1987); note = Kozak sequence; regulatory_class = other; standard_name = Kozak sequence
  CDS 3163..3165: note = start, codon_start = 1; translation = M
  misc_feature 3232..3252: note = Landing pad
  misc_feature 3253..3255: note = protospacer adjacent motif (PAM)
  misc_feature 3256..3276: note = SV40NLS
  CDS 3277..3993: note = codon_start = 1, mammalian codon-optimized (SEQ ID NO: 29); product = The original enhanced GFP (Yang et al., 1996); protein_id = 48; standard_name = no-ATG-eGFP; translation = VSKGEELFTGVVPILVELDGDVNGHKFSVSGEGEGDATYGKLTL KFICTTGKLPVPWPTLVTTLTYGVQCFSRYPDHMKQHDFFKSAMPEGYVQERTIFFKD DGNYKTRAEVKFEGDTLVNRIELKGIDFKEDGNILGHKLEYNYNSHNVYIMADKQKNG IKVNFKIRHNIEDGSVQLADHYQQNTPIGDGPVLLPDNHYLSTQSALSKDPNEKRDHM VLLEFVTAAGITLGMDELYK
  misc_feature 4006..4766: note = CBh
  CDS 4779..5486: note = codon_start = 1, mammalian codon-optimized (SEQ ID NO: 32); product = monomeric derivative of DsRed fluorescent protein (Shan er et al., 2004); protein_id = 46; standard_name = mCherry; translation = MVSKGEEDNMAIIKEFMRFKVHMEGSVNGHEFEIEGEGEGRPYE GTQTAKLKVTKGGPLPFAWDILSPQFMYGSKAYVKHPADIPDYLKLSFPEGFKWERVM NFEDGGVVTVTQDSSLQDGEFIYKVKLRGTNFPSDGPVMQKKTMGWEASSERMYPEDG ALKGEIKQRLKLKDGGHYDAEVKTTYKAKKPVQLPGAYNVNIKLDITSHNEDYTIVEQ YERAEGRHSTGGMDELYK
  regulatory 4779..4782: note = Kozak sequence; note = vertebrate consensus sequence for strong initiation of translation (Kozak, 1987); regulatory_class = other; standard_name = Kozak sequence
  misc_feature 5552..6140: note = woodchuck hepatitis virus posttranscriptional regulatory element (WPRE)
  misc_feature 6023..6034: note = CDS complement, Factor Xa recognition and cleavage site, codon_start =1, translation: IEGR
  misc_feature 6228..6461: note = 3' LTR (Delta-U3), self-inactivating 3' long terminal repeat (LTR) from HIV- 1
  polyA_site 6533..6654: note = SV40 polyadenylation (poly(A)) signal
  rep_origin 6694..6829: note = SV40 origin of replication
  misc_feature 6850..6868: note = promoter complement, T7 promoter, promoter for bacteriophage T7 RNA polymerase
  rep_origin 7036..7491: direction = right; note = f1 bacteriophage origin of replication; arrow indicates direction of (+) strand synthesis
  regulatory 7517..7621: note = AmpR promoter; regulatory_class = promoter
  CDS 7622..8482: gene = bla; note = codon_start =1, confers resistance to ampicillin, carbenicillin, and related antibiotics (SEQ ID NO: 33); product = beta-lactamase; protein_id = 47; translation = MSIQHFRVALIPFFAAFCLPVFAHPETLVKVKDAEDQLGARVGY IELDLNSGKILESFRPEERFPMMSTFKVLLCGAVLSRIDAGQEQLGRRIHYSQNDLVE YSPVTEKHLTDGMTVRELCSAAITMSDNTAANLLLTTIGGPKELTAFLHNMGDHVTRL DRWEPELNEAIPNDERDTTMPVAMATTLRKLLTGELLTLASRQQLIDWMEADKVAGPL LRSALPAGWFIADKSGAGERGSRGIIAALGPDGKPSRIVVIYTTGSQATMDERNRQIA EIGASLIKHW
  rep_origin 8653..9241: direction = right; note = high-copy-number ColE1/pMB1/pBR322/pUC origin of replication
  protein_bind 9529..9550: bound_moiety = E.coli catabolite activator protein; note = CAP binding site - CAP binding activates transcription in the presence of cAMP
  regulatory 9565..9595: note = promoter for the E. coli lac operon; regulatory_class = promoter
  protein_bind 9603..9619: bound_moiety = lac repressor encoded by lacl; note = The lac repressor binds to the lac operator to inhibit transcription in E. coli. This inhibition can be relieved by adding lactose or isopropyl-beta-D-thiogalactopyranoside (IPTG).
  regulatory 9664..9682: note = promoter for bacteriophage T3 RNA polymerase (SEQ ID NO: 34); regulatory_class = promoter
  misc_feature 1..9707: note = SuperCAR Expression Vector Sequence with TRAC molecular switch and eGFP cassette
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcatatgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccccgtacggccaccatgggaagcggagccacgaacttctctctgttaaagcaagcaggagatgttgaagaaaaccccgggcctttcggaacccaatcactngacaggccaaagaagaagcgtaaggtagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggatatcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagttaattaagtcgactctagaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 17 - DNA, 10430 bp, synthetic construct
  source 1..10430: mol_type = other DNA
  regulatory 3..229: note = Rous sarcoma virus (RSV) enhancer/promoter; regulatory_class = promoter
  misc_feature 230..410: note = 5'LTR (truncated) - truncated 5' long terminal repeat (LTR) from HIV-1
  misc_feature 457..582: note = HIV-1 Psi - packaging signal of human immunodeficiency virus type 1
  misc_feature 1075..1308: note = RRE, The Rev response element (RRE) of HIV-1 allows for Rev-dependent mRNA export from the nucleus to the cytoplasm
  misc_feature 1803..1920: note = central polypurine tract (cPPT) and central termination sequence (CTS) of HIV-1
  regulatory 1965..2930: note = EF-1-alpha promoter, strong constitutive promoter for human elongation factor EF-1-alpha; regulatory_class = promoter
  intron 2196..2930: note = EF-1-alpha intron A,intron upstream of the start codon of humanEF-1-alpha
  intron 2931..3134: note = EF-1-alpha intron A, truncated intron upstream of the start codon of human EF- 1-alpha
  regulatory 3157..3162: note = Kozak sequence, vertebrate consensus sequence for strong initiation of translation (Kozak, 1987); regulatory_class = other; standard_name = Kozak sequence
  CDS 3163..3165: note = start; translation = M
  misc_feature 3232..3252: note = TRAC Guide
  misc_feature 3256..4716: note = CAR
  misc_feature 4729..5489: note = CBh
  CDS 5502..6209: note = mammalian codon-optimized (SEQ ID NO: 32), codon start 1; product = monomeric derivative of DsRed fluorescent protein (Shaner et al., 2004); protein_id = 49; translation = MVSKGEEDNMAIIKEFMRFKVHMEGSVNGHEFEIEGEGEGRPYE GTQTAKLKVTKGGPLPFAWDILSPQFMYGSKAYVKHPADIPDYLKLSFPEGFKWERVM NFEDGGVVTVTQDSSLQDGEFIYKVKLRGTNFPSDGPVMQKKTMGWEASSERMYPEDG ALKGEIKQRLKLKDGGHYDAEVKTTYKAKKPVQLPGAYNVNIKLDITSHNEDYTIVEQ YERAEGRHSTGGMDELYK
  regulatory 5502..5505: note = vertebrate consensus sequence for strong initiation of translation (Kozak, 1987); regulatory_class = other; standard_name = Kozak sequence
  misc_feature 6275..6863: note = woodchuck hepatitis virus post Transcriptional regulatory element (WPRE)
  misc_feature 6746..6757: note = CDS complement, Factor Xa site, Codon start =1, translation: IEGR; product = Factor Xa recognition and cleavage site
  misc_feature 6951..7184: note = 3' LTR (Delta-U3), self-inactivating 3' long terminal repeat (LTR) from HIV- 1
  polyA_site 7256..7377: note = SV40 polyadenylation (poly(A)) signal
  rep_origin 7417..7552: note = SV40 origin of replication
  misc_feature 7573..7591: note = T7 promoter, promoter for bacteriophage T7 RNA polymerase
  rep_origin 7759..8214: direction = right; note = f1 bacteriophage origin of replication; arrow indicates direction of (+) strand synthesis
  regulatory 8240..8344: note = bla, AmpR promoter; regulatory_class = promoter
  CDS 8345..9205: gene = bla; note = codon_start=1, AmpR, confers resistance to ampicillin, carbenicillin, and related antibiotics (SEQ ID NO 33); product = beta-lactamase; protein_id = 52; translation = MSIQHFRVALIPFFAAFCLPVFAHPETLVKVKDAEDQLGARVGY IELDLNSGKILESFRPEERFPMMSTFKVLLCGAVLSRIDAGQEQLGRRIHYSQNDLVE YSPVTEKHLTDGMTVRELCSAAITMSDNTAANLLLTTIGGPKELTAFLHNMGDHVTRL DRWEPELNEAIPNDERDTTMPVAMATTLRKLLTGELLTLASRQQLIDWMEADKVAGPL LRSALPAGWFIADKSGAGERGSRGIIAALGPDGKPSRIVVIYTTGSQATMDERNRQIA EIGASLIKHW
  rep_origin 9376..9964: direction = right; note = high-copy-number ColE1/pMB1/pBR322/pUC origin of replication
  protein_bind 10252..10273: bound_moiety = E. coli catabolite activator protein; note = CAP binding site, CAP binding activates transcription in the presence of cAMP
  regulatory 10288..10318: note = lac promoter, promoter for the E. coli lac operon; regulatory_class = promoter
  protein_bind 10326..10342: bound_moiety = lac repressor encoded by lacI; note = lac operator, The lac repressor binds to the lac operator to inhibit transcription in E. coli. This inhibition can be relieved by adding lactose or isopropyl-beta-D-thiogalactopyranoside (IPTG)
  regulatory 10387..10405: note = T3 promoter, promoter for bacteriophage T3 RNA polymerase (SEQ ID NO: 34); regulatory_class = promoter
  misc_feature 1..10430: note = SuperCAR Expression Vector Sequence with TRAC molecular switch and CAR cassette
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcatatgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccccgtacggccaccatgggaagcggagccacgaacttctctctgttaaagcaagcaggagatgttgaagaaaaccccgggcctttcggaacccaatcactngacaggatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggatatcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagttaattaagtcgactctagaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 18 - DNA, 10043 bp, synthetic construct
  source 1..10043: mol_type = other DNA
  regulatory 3..229: note = Rous sarcoma virus (RSV) enhancer/promoter; regulatory_class = promoter
  misc_feature 230..410: note = 5' LTR (truncated), truncated 5' long terminal repeat (LTR) from HIV-1
  misc_feature 457..582: note = HIV-1 Psi, packaging signal of human immunodeficiency virus type 1
  misc_feature 1075..1308: note = RRE, The Rev response element (RRE) of HIV-1 allows for Rev-dependent mRNA export from the nucleus to the cytoplasm
  misc_feature 1803..1920: note = central polypurine tract (cPPT) and central termination sequence (CTS) of HIV-1
  regulatory 1965..2930: note = EF-1-alpha promoter, strong constitutive promoter for human elongation factor EF-1-alpha; regulatory_class = promoter
  intron 2196..2930: note = EF-1-alpha intron A, intron upstream of the start codon of human EF-1-alpha
  intron 2931..3134: note = EF-1-alpha intron A, truncated intron upstream of the start codon of human EF-1-alpha
  regulatory 3157..3162: note = vertebrate consensus sequence for strong initiation of translation (Kozak, 1987); note = Kozak sequence; regulatory_class = other; standard_name = Kozak sequence
  CDS 3163..3165: note = start, codon_start =1; translation = M
  misc_feature 3232..3252: note = Landing Pad, TRAC guide
  misc_feature 3253..3255: note = protospacer adjacent motif (PAM)
  misc_feature 3256..4329: note = HLA-E
  misc_feature 4342..5102: note = CBh
  CDS 5115..5822: note = mCherry, codon_start=1, mammalian codon-optimized" (SEQ ID NO: 32); product = monomeric derivative of DsRed fluorescent protein (Shaner et al., 2004); protein_id = 51; translation = MVSKGEEDNMAIIKEFMRFKVHMEGSVNGHEFEIEGEGEGRPYE GTQTAKLKVTKGGPLPFAWDILSPQFMYGSKAYVKHPADIPDYLKLSFPEGFKWERVM NFEDGGVVTVTQDSSLQDGEFIYKVKLRGTNFPSDGPVMQKKTMGWEASSERMYPEDG ALKGEIKQRLKLKDGGHYDAEVKTTYKAKKPVQLPGAYNVNIKLDITSHNEDYTIVEQ YERAEGRHSTGGMDELYK
  regulatory 5115..5118: note = vertebrate consensus sequence for strong initiation of translation (Kozak, 1987); note = Kozak sequence; regulatory_class = other; standard_name = Kozak sequence
  misc_feature 5888..6476: note = woodchuck hepatitis virus posttranscriptional regulatory element (WPRE)
  misc_feature 6359..6370: note = Factor Xa site, codon start = 1, translation: IEGR; product = Factor Xa recognition and cleavage site
  misc_feature 6564..6797: note = 3' LTR (Delta-U3), self-inactivating 3' long terminal repeat (LTR) from HIV- 1
  polyA_site 6869..6990: note = SV40 polyadenylation (poly(A)) signal
  rep_origin 7030..7165: note = SV40 origin of replication
  regulatory 7186..7204: note = T7 promoter, promoter for bacteriophage T7 RNA polymerase; regulatory_class = promoter
  rep_origin 7372..7827: direction = right; note = f1 bacteriophage origin of replication; arrow indicates direction of (+) strand synthesis
  regulatory 7853..7957: gene = bla; note = AmpR promoter; regulatory_class = promoter
  CDS 7958..8818: gene = bla; note = AmpR,codon_start=1, confers resistance to ampicillin, carbenicillin, and related antibiotics" (SEQ ID NO: 33); product = beta-lactamase; protein_id = 50; translation = MSIQHFRVALIPFFAAFCLPVFAHPETLVKVKDAEDQLGARVGY IELDLNSGKILESFRPEERFPMMSTFKVLLCGAVLSRIDAGQEQLGRRIHYSQNDLVE YSPVTEKHLTDGMTVRELCSAAITMSDNTAANLLLTTIGGPKELTAFLHNMGDHVTRL DRWEPELNEAIPNDERDTTMPVAMATTLRKLLTGELLTLASRQQLIDWMEADKVAGPL LRSALPAGWFIADKSGAGERGSRGIIAALGPDGKPSRIVVIYTTGSQATMDERNRQIA EIGASLIKHW
  rep_origin 8989..9577: direction = right; note = high-copy-number ColE1/pMB1/pBR322/pUC origin of replication
  protein_bind 9865..9886: bound_moiety = E. coli catabolite activator protein; note = CAP binding site, CAP binding activates transcription in the presence of cAMP
  regulatory 9901..9931: note = lac promoter, promoter for the E. coli lac operon; regulatory_class = promoter
  protein_bind 9939..9955: bound_moiety = lac repressor encoded by lacI; note = lac operator, The lac repressor binds to the lac operator to inhibit transcription in E. coli. This inhibition can be relieved by adding lactose or isopropyl-beta-D-thiogalactopyranoside (IPTG).
  regulatory 10000..10018: note = T3 promoter, promoter for bacteriophage T3 RNA polymerase (SEQ ID NO: 34); regulatory_class = promoter
  misc_feature 1..10043: note = SuperCAR Expression Vector Sequence with TRAC molecular switch and CAR (HLA-E) cassette
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcatatgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccccgtacggccaccatgggaagcggagccacgaacttctctctgttaaagcaagcaggagatgttgaagaaaaccccgggcctttcggaacccaatcactngacagggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggatatcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagttaattaagtcgactctagaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 19 - DNA, 9761 bp, synthetic construct
  source 1..9761: mol_type = other DNA
  misc_feature 1..9761: note = SuperCAR Expression Vector Sequence with TRAC molecular switch and Neomycin Resistance cassette
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcatatgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccccgtacggccaccatgggaagcggagccacgaacttctctctgttaaagcaagcaggagatgttgaagaaaaccccgggcctttcggaacccaatcactngacaggcttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgcactcggatggaagccggtcttgtcgatcaggatgatctggacgaagagcatcaggggctcgcgccagccgaactgttcgccaggctcaaggcgcgtatgccggatggtgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggatatcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaaaaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagttaattaagtcgactctagaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 20 - DNA, 10228 bp, Homo sapiens
  source 1..10228: mol_type = genomic DNA
  intron 1..600
  exon 601..746
  intron 747..6528
  exon 6529..6888
  intron 6889..7155
  exon 7156..7311
  intron 7312..7526
  exon 7527..7560
  intron 7561..8211
  exon 8212..9628
  intron 9629..10228
  misc_feature 1..10228: note = PD1 gene sequence, ENSG00000188389, taken from ensembl.org
cgattagccatggacagttgtcattcagtagggtcacctgtgccccagcgaagggggatgggccgggaaggcagaggccaggcacctgcccccagcaggggcagaggctgtgggcagccgggaggctcccagaggctccgacagaatgggagtggggttgagcccacccctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggagttctgtcctgtctggtggctgtgggggcagtgggcaggagggagggaggcctccagggagggacaaggcggcagccaggctgaggctgtgtgtggagggtgctggtggctcccctggatcctggcctgagtcaggccttcccctcgctgtcccagaaacacacaggccctcctcgccctgtttgccccactcacatgtagagccctccggtgaaaaattcctccaacatcacggggctggggacaccatcttccccaagcccaggccttttaggggggtcccacggccccctggccccaggcttcctggccaggcatgggtgtccctgagtgactgtgcctgctccatagcccccgtgtccccatgcctaccagtccctccgggcaggggtggtacctggtgccagggcgtccacagcggggtgggagggctgccctgggctatgagggacacagcatctgcgggtccctccctaagtacacagggtgggggcagcgggggaggcggggtcacgtctgctggagccctatcaccctgctgcggtccttggcagctagcagcagggacggtgcagggaaaggagccctgccaggccctcg

SEQ ID NO: 21 - DNA, 2908 bp, Homo sapiens
  source 1..2908: mol_type = genomic DNA
  intron 1..600
  exon 601..987
  intron 988..1503
  exon 1504..1521
  intron 1522..1664
  exon 1665..1771
  intron 1772..2062
  exon 2063..2308
  intron 2309..2908
  misc_feature 1..2908: note = TRBC2 gene sequence, ENSG00000211772, taken from ensembl.org
tcccagattccccttcattcctgagcaccttagatttggtataaataacctgcttgggagggggctttttgaattcgtacataatttaaccttcacacagtttctgcaaagtcagaatggtgattattacctcacatgcagaaaaaagtgataggaatttctgtcttaaaagtcttgttggtggacaaaggaagttctaggatttggatcttgtttttttgggttccaatcccttgctccagttaaaaaactaccacataaaatggtgagaagtaggtaggcaagtttttattgatagagaggaaatcaaataatggcaatgaggagacatcacctggaatgttaggcagtgcctaactgggggatggacagacaatgggcagtgccaacccatagggtggatacaaaagacaggcaaggaaggggtagaaccatcaaagaggaataggctggtgaccccaaagcaaggaggacctagtaacataattgtgcttcattatggtcctttcccggccttctctctcacacatacacagagcccctaccaggaccagacagctcttagagcaaccctagccccattacctcttccctttccagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggtgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatttcaggagtgtctgtggagttctgctcatcactgacctatcttctgatttagggaaagcagcattcccttggacatctgaagtgacagccctctttctctccacccaatgctgctttctcctgttcatcctgatggaagtcctcaaacaccatttccatacccaggcattctgggtccccactggagggttagtctgaagggcaatggctgggctttggaaaaccagcaagatgaggacagagaggaaggcacacagcaaaccataagcccttacccagtgcaggacagaggatgcgggcagacctatgggttacaatgtctggtcatttcccaattccagattaaactgtcacctgttttacctttagttttattagtttgtagtcttaacacctccagcttctcttgtttcaggatttgggcttaaaattgagtgctactctgcatgtctagtttgaaatactagagaaggcagagttgagacaactgatatgtaaagcctggggaagagtgatttctcaggagcgagacacactaagtcaggagcaatgggatatagggcccagtgggggctgaagtgctatgttcagagtagcccttc

SEQ ID NO: 22 - DNA, 20 bp, synthetic construct
  source 1..20: mol_type = other DNA
  misc_feature 1..20: note = Target sequence of nucleic acid guide #4 (in PD1)
tccagagagaagggcagaag

SEQ ID NO: 23 - RNA, 20 nt, synthetic construct
  source 1..20: mol_type = other RNA
  misc_feature 1..20: note = Nucleic acid guide #3 (against PD1)
cagcggcacctacctctgtg

SEQ ID NO: 24 - RNA, 20 nt, synthetic construct
  source 1..20: mol_type = other RNA
  misc_feature 1..20: note = Nucleic acid guide #4 (against PD1)
cttctgcccttctctctgga

SEQ ID NO: 25 - RNA, 20 nt, synthetic construct
  source 1..20: mol_type = other RNA
  misc_feature 1..20: note = Nucleic acid guide #5 (against TCRbeta1/TCRbeta2)
tgggaaggaggtgcacagtg

SEQ ID NO: 26 - RNA, 20 nt, synthetic construct
  source 1..20: mol_type = other RNA
  misc_feature 1..20: note = Nucleic acid guide #6 (against TCRbeta1/TCRbeta2)
aaaggccacactggtgtgcc

SEQ ID NO: 27 - DNA, 20 bp, synthetic construct
  source 1..20: mol_type = other DNA
  misc_feature 1..20: note = Target sequence of nucleic acid guide #5 (in TCRbeta1/TCRbeta2)
cactgtgcacctccttccca

SEQ ID NO: 28 - DNA, 20 bp, synthetic construct
  source 1..20: mol_type = other DNA
  misc_feature 1..20: note = Target sequence of nucleic acid guide #6 (in TCRbeta1/TCRbeta2)
ggcacaccagtgtggccttt

SEQ ID NO: 29 - DNA, 20 bp, synthetic construct
  source 1..20: mol_type = other DNA
  misc_feature 1..20: note = Target sequence of nucleic acid guide #7 (in TRAC)
atcactgacaggttttgaaa

SEQ ID NO: 30 - DNA, 20 bp, synthetic construct
  source 1..20: mol_type = other DNA
  misc_feature 1..20: note = Target sequence of nucleic acid guide #8 (in PD-1)
ctcagccgtgcctgtgttct

SEQ ID NO: 31 - protein, 238 aa, synthetic construct
  UNSURE 1..238: note = eGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 32 - protein, 236 aa, synthetic construct
  UNSURE 1..236: note = mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 33 - protein, 286 aa, synthetic construct
  UNSURE 1..286: note = AmpR
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 34 - DNA, 10043 bp, synthetic construct; identical to SEQ ID NO: 18
  source 1..10043: mol_type = other DNA
  misc_feature 1..10043: note = T3 promoter

SEQ ID NO: 35 - DNA, 2875 bp, synthetic construct
  source 1..2875: mol_type = other DNA
  intron 1..600
  exon 601..987
  intron 988..1428
  exon 1429..1446
  intron 1447..1598
  exon 1599..1705
  intron 1706..2027
  exon 2028..2275
  intron 2276..2875
  misc_feature 1..2875: note = TRBC1 gene sequence, ENSG00000211751, taken from ensembl.org
aaagagggttacatgcaagaaaataaaattttttaaaaaggtgctgggcaggtgggggactcagatgtaatggaaaagtgtcttttctagaaaagaaaagctaattctaatatgtgtcactaccccacgagacaaatatatacatcttgatttaaaaaaggaaaattataattagaaaaagtcaatttagttattgtaattataccactaatgagagtttcctacctcgagtttcaggattacatagccatgcaccaagcaaggctttgaaaaataaagatacacagataaattatttggatagatgatcagacaagcctcagtaaaaacagccaagacaatcaggatataatgtgaccataggaagctggggagacagtaggcaatgtgcatccatgggacagcatagaaaggaggggcaaagtggagagagagcaacagacactgggatggtgaccccaaaacaatgagggcctagaatgacatagttgtgcttcattacggcccattcccagggctctctctcacacacacagagcccctaccagaaccagacagctctcagagcaaccctggctccaacccctcttccctttccagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccagccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgactctgacttgtgaatgtctggatagctccttggctgtctctgaactccctgtgactctccccattcagtcaggatagaaacaagaggtattcaaggaaaatgcagactcttcacgtaagagggatgaggggcccaccttgagatcaatagcagaggttaattcagcgtgaaaggcagtgatgggagctgaagaggttacttctagaacagtctaggaagacacagatgttgagtataggaattttctatatccaactatactgttctgcccaggaaagacgtgctcagaggaagagccaacctatacaggtgtgttcaccctccagctggccatgtccccgtgactaacaaagctggattccataagcatcacccaccttccttgcagctttcttattgagcactccattcatcttcattggttcaccaagttgatttcccagctccaaagaagagaggctctgacttgcaaacttattttcaatggaagatgtgtcttccggtttaagttacccatctgtttataaatctctctctagtgaattaaaccagaatgaaaatgtcccctaatcattcctggaaggttagaaaataaaggta

SEQ ID NO: 36 - RNA, 20 nt, synthetic construct
  source 1..20: mol_type = other RNA
  misc_feature 1..20: note = Nucleic acid guide #7 (against TRAC)
tttcaaaacctgtcagtgat

SEQ ID NO: 37 - RNA, 20 nt, synthetic construct
  source 1..20: mol_type = other RNA
  misc_feature 1..20: note = Nucleic acid guide #8 (against PD1)
agaacacaggcacggctgag

SEQ ID NO: 38 - DNA, 20 bp, synthetic construct
  source 1..20: mol_type = other DNA
  misc_feature 1..20: note = TCRbeta2 forward primer
tggtcctttcccggccttct

SEQ ID NO: 39 - DNA, 20 bp, synthetic construct
  source 1..20: mol_type = other DNA
  misc_feature 1..20: note = TCRbeta2 reverse primer
atctccccaggccccactca

SEQ ID NO: 40 - DNA, 36 bp, synthetic construct
  source 1..36: mol_type = other DNA
  misc_feature 1..36: note = Stuffer sequence
gtactaacaagttgtatggtactaacaagttgtatg

SEQ ID NO: 41 - RNA, 20 nt, synthetic construct
  source 1..20: mol_type = other RNA
  misc_feature 1..20: note = Nucleic acid guide #9 (against TIM342)
tgtgtttgaatgtggcaacg

SEQ ID NO: 42 - DNA, 20 bp, synthetic construct
  source 1..20: mol_type = other DNA
  misc_feature 1..20: note = Target sequence of nucleic acid guide #9 (in TIM342)
cgttgccacattcaaacaca

SEQ ID NO: 43 - DNA, 21 bp, synthetic construct
  source 1..21: mol_type = other DNA
  misc_feature 1..21: note = TIM342 forward primer
agcgaatcatcctccaaacag

SEQ ID NO: 44 - DNA, 22 bp, synthetic construct
  source 1..22: mol_type = other DNA
  misc_feature 1..22: note = TIM342 reverse primer
tggggcctgttaaactttaggt

SEQ ID NO: 45 - DNA, 58646 bp, Homo sapiens
  source 1..58646: mol_type = genomic DNA
  intron 1..410
  exon 411..650
  intron 651..694
  exon 695..1069
  intron 1070..4021
  exon 4022..6046
  intron 6047..28568
  exon 28569..28696
  intron 28697..30273
  exon 30274..30373
  intron 30374..33720
  exon 33721..34534
  intron 34535..36119
  exon 36120..37349
  intron 37350..38534
  exon 38535..38681
  intron 38682..38719
  exon 38720..38803
  intron 38804..44270
  exon 44271..44610
  intron 44611..48008
  exon 48009..48778
  intron 48779..54490
  exon 54491..54527
  intron 54528..56173
  exon 56174..58046
  intron 58047..58646
  misc_feature 1..58646: note = TIM342 gene sequence, ENSG00000135077, taken from ensembl.org
gttgcagcgagccgaaatcgcgccactgcagtccagcctgggcgaaagagaaagactccgtctcaaagaaaaaaacaacaaaaaaaccctgcatgtctatattttcgaatttgtatgtaagacgtcttaattgtatgcgttaaattaaaaaaggacaaaaggaatggaactaccagcagcgactaagtcagtaaaagagtggtaatggggaagggggtggaatatacgcaggcgcaatagcaagcgtttcttttggaggttattttcgtgtcataaatttctaaattcacgttaagtcttacaagagcatgttaaacattaaaacataagattgtttctggtcaataaggggtctataaaaaataaagcaataaggtgtcttgttcaattttatgccttaattcaaaaagaaacgaagaaacggaaccctcagctacgacttttaaaagtcagtaacgggtggatgggaggagtcaaacatacgtaggcgcaacagcgagggatcggtctgaggcggagctagaatttcgttaaaggacagctcttacagccaatcaaggacagagtcggtggcttcttcccggaggagaccggaagttggtttcggctgcagaggggaaggcggctaccagtgtaaagccagagctgaggtaagtgggcggcaacggtttggggccgccgcctgtgtgcgtttttttcttccggaccccgcagcttcctctgtgctgggctgggaactggggaggcatggctgagttcggcctccaaaggcggtctttcgtctttctctcccgagtctctccgagagtgcggtctctccgagagcgcggaggcctcggggtggacagcctggaggagggggctctaagcgtggcagtctcggggcttcctccgtgttgggacgccgagccggagtggaggttgggagacctggcgtctagtccccgcgccgctgtttttctaagctgtgtggccttgggcgagtcatttgccctctccggctctttgtaacatgagagtagccctccctgcccttccttcttctgggagcagttatgaaacagctgaggtatgggggcaggcgagggcggtagatcccaagcctaatcgtcaggtccatctggagaggttttaaaagcgacattctagggcccattcacagtagacagaatgaatcaaatctctggggctgggaccctggaatgtctgtcgtcttctggtgaagctccttctcccccagtagaacaaacactagatgttggatcttgagttaagaaccagctctgatgcttggctctgcttttattaaaaataagacctccatcacctttgaaaagttccatctagagagacagtagagaaagaaatataatttttgtgatatgccagtccttattgtacagttagatgcttctcggcttagggatataagagagcttgccctttggagctaaagggacctgggataagaattcctgttgtgccatttattaaccttttgatctggggcaacacttcccctcattgagacttcggatcctcacttctaaaatgaaattgataatagaacttaattcgtgggaccgtgtggtggctcacgcctgtaatcccagcactttgggaggccgaggtgggcggatcacttgaggtcaggagttccagaccagcctggccaacatggtgaaaacctgtctttaccgagaaatacaaaaattagctgggcgtggtggtgcgcacctgtagtccccagctactcaggaggctcaggcaggagaatcccttgaacccgggaggcagaggttgcagtgagccctgatggtaccactgcactccagcctgggagacagattgagcccttgtctcaaaaaacaaaactaacaacaaaaaccccaaaaaccttaattcatagaattgttaatgaagattatgttacagaggccataaaaaatggtacctgtcatgtagttggccttaggaaaatggcagccgcaatacaggaatgcacatcgtgtaggaaagtacagagcagcactcctcacctaaaaaggtttgagtgggatgaaacgtgagctgtcaaaaattgtattttctgccgggcacggtggctcacgcctgtaatcccagaactttgggaggtcgaggcaggtggaacacgaggtcaggagatcgagaccatcctggctaacacgatgaaatctcgtctctactgaaaatagaaaacattagccgggcgtcgtggcgggcgcctgtagtcctagctactcaggcggctgaggcaggagaatggcgtgaacccgggagtccgagcttgcagtgagccgagatcgtgccactgcactccaacctgggtgacagagcgagacactgtctcagggaaaaaaaaaatgtattttgaagtaccagatcactgttaattctttgattttggtaattgtgctttgggtagaagaaaacattcttgtcacgagaaaacacactgaagtattcaggggtaaaggaatgaaaaaccaataaagcaaatgaaggaaaatgttaacatttggagaatctgagtgaagaatatgaagtgttttttgtgtattcatgcactttttctgtaatctgaatttatgttaaaagttttaaaatgtctgggtgtggtggctcacgcctgtaatcccagcactttgggaggccaaggcaggaggattgcttgagctcaggagtttgaaacaagcctgagtaacatggcaaaactctgtctctacaaaatatacaaaaattagccgggcttggtgacatgcctgtagtcccagctgcttgggaggttgaggtgggaggatcgcttgagcctaggagggcgaggctgcatcatcctatgcaccactgtggcatagtggaaactcagggctgcgccacagctgccctcagtacctcacgaattctaagtataatattgcttctagtaggatacttgccccacccgatgttagagtgtgttatatcttgaaactctgataacaccaatctaaaggagcacttaaggatcttctcattggatggtatacctaggcttaaggtagaataagtagaggttattatggccatagatactgggtttgaacctggggtttgacatttctgtgatagttgtagagaagttaatgtgtccatacaggccaggcgtggtggctcacgcctgtaatcccagcactttgggaggccaaggtgggcggatcacctgagatggggagtaaagagaccaacctggccaacatggcaaaatcccgtctctactaaaaatacaaaaattagccagacatggtgcacgcctgtaatcccatctacttaggaggctgaggcaggagaattgcttgagcccaggaggcggaggttgcagtgagctgagatctgagattgcaccactgcactctagcctggacatcagagcaagactccatctcaaaaaacaaacaaaagttaatgtgtccatataaaattgcatagtatactgtgacaaaaagggtatggaactgaatcatcatttcattcaggaactttggataataatgtgtttaggatttagcaatgttaattttgtcagagtgtttatgagagatacaagtaagtgtttgctattggaaataaatctataacataaaacaaaagtgattgtgatatgagattgggagacagcctgtgtagtttggttatggaagagtaaagaaatggggcataagtggtgaggggcatggagtccagaagattttctttagagatgagaggatgctacatattttttaatgctgatgaaattattagagagagaatggaaaatatgtgaatggttgagaatataatgcctacttagaaaagatggcagactttccctatcataaatgttagttttagaaaccaaggagatcatcttgtcttcggccccaaactgctggaagaaaaggaagaaaatattccatctgtagttatgaaatgtaacaggtttgtcttgtggctctaataaatctttggtctgtgtccataggttcttgatagtccacaatgggtgaaccacagcaagtgagtgcacttccaccacctccaatgcaatatatcaaggaatatacggatgaaaatattcaagaaggcttagctcccaagcctccccctccaataaaagacagttacatgatgtttggcaatcagttccaatgtgatgatcttatcatccgccctttggaaagtcagggcatcgaacggcttcatcctatgcagtttgatcacaagaaagaactgagaaaacttaatatgtctatccttattaatttcttggaccttttagatattttaataaggagccctgggagtataaaacgagaagagaaactagaagatcttaagctgctttttgtacacgtgcatcatcttataaatgaataccgaccccaccaagcaagagagaccttgagagtcatgatggaggtccagaaacgtcaacggcttgaaacagctgagagatttcaaaagcacctggaacgagtaattgaaatgattcagaattgcttggcttctttgcctgatgatttgcctcattcagaagcaggaatgagagtaaaaactgaaccaatggatgctgatgatagcaacaattgtactggacagaatgaacatcaaagagaaaattcaggtcataggagagatcagattatagagaaagatgctgccttgtgtgtcctaattgatgagatgaatgaaagaccatgaaagatgtttctttttctttttttccttttgataatagcatcatatattagttcattttcttttggacagtcttaagagaagtttcactaaaaatgtaaacagctttaatcttgactccaaatttttcaattatgagatgtcataggcagtaatttcgctgtataacaagcatagacaaatgagtgtccctgcactaagaagaatcactttaaaaagcaaagtgttagctgctgttgtatgggacattcctatgttttagagttgcagtaaaactttgatgataacctcaataatagcaaagttttcgtctttgaaaaggggatttagcatttgctttaagaatgatagataaatggatattaagctctctacatgtaaaactatgaaatctttagacttattccattaaaaattttgcttaagctccaaaaagtagcataacatgttgatagagaggagcccagtagagttataaaatagaaacttcattttttcctcatgactgcttctgtaaacccactagctcagtcttttctccctatcctgaatggactcttgcagggaagtcccataaattgttgtttttttttccagtcactcaggtaataagtcctttggtcactttaagttacagacattaattttagtaattaagatgtctgtgtcattagctatacattttctttaaagctaagctcagacttgtttaagtccacaatgggaaaggggtttgatggcagagactgctaggtgtctaccaaagattcttgctctttgaagatatgttgtcaagatgtggcaatccagccagggacatttccctactcccagcatctctgtgagaacatatgactaagtcgtaggcggtgaaatgtgggcataggaatccaggcttgccccataaacatttcttgcatgatcctccattctcttctcccagctgctggttggacggtcgtatccaggataagtttggaagccaccgaagatggcaaagattgtcagtctggttctttgaatgaccgtgaggactaagtcttcctttcagtaactccactctgcccctagctaattggactttatatgagcactttcttctcttcttcccccgactgtccatctgctcttccaccaaggtgttagtggagagttgaccttgtgtctgaggatctctgcctgccgttttcttaatgtaggcccgtggttcttaaagtgtgtggtcctggaccagcagcattggtatcacttggggacttactagaaataaaagttttagtataatccgtaggttcagaaaaataaaaaataaatgagggttctctggctccatcccagaccttttgaaccagaaacttctgggtgtaggtcccagcaagctgtattttaagaagcctatgtgattatgatacatgctcaagattaagaaacattgaggctgggtgcagtggcccactcctgtaatcccagcacttcgggaggctgaggctggtggatcacctgaagtcaggagttcgagaccagcctgaccaacatggggaaatcccatctctactaaaatacaaaattagctgggcttggtggcgcatgcctgtaatcccagctactcgggaggctgaggcagggagaatcacttgaatctgggaggcagaggttgtggtgagctgagattgcaccattgcactccagcctaggcaacaagagcgaaactccatctcaaaaacaaaaaagaaccattgagaggcaatgcctttccccagatgtctacttttttatttcttccttagcccctggtttctagcatagggatttgctcacttaaactttcattggtcctagagctagcaaagttcacattaaaatgattataaggggcctggcgcagtggctcatgcctgtaatcctagcactttgggaggctgaggtgggtggattgcttgaggtcaggagtttgataccagcctggccaacatggcaaaaccccgtctgtactaaaaatacaaaaattagccgggcatagtggcaggcgcctgtaatcccagctactcgggaggctgaggcacgagaattccttgaacccaaggggtggaggttgcagtgagccgagatcgtgccactgcactccagcctgggtgacagcaaaactctgtctaaaatatatatatatatgtgtatgtatgtatatatgtatgactataggtcagttctctatgaaatgtgcatgctgtgctttcacagatccagggactgtaagttatccctaggtagacacttctttatcagataggcagttggttggctagtctcaacttttgtttttttctggtacgtaatgtgtttggtatagacctttgggcctcagagactccagaggacatagttcaggctctcaagcttaaattgaagtagggagcatcaccttgtgagggagaaaggtggaaattgcttagcacttgaacaactctccaaagaaaattctctttctttgactctgagaaccttgaggaaggtgttgggttgaggtgcaaaaccagtttttggccactttgaaaatctttgcatgtaaaaaacagaatcacataagagccacaacactacttatatatgaacgtgtgtgtgttggggggaggggcgttgagggattaaaatgccactggggggccaggcacagtggcttatgcctgtaatcccagcactttgggaggctgaggtaggaggatggttttaggtcaggagtttgagactagcctaggcaacatagtgagaccgtgtctctacaacaaattagaaaaaaaaatgccattggggaggatctgatcttgcaggtctgtgactaggacctgacataaattcctaaagtgttctgatatgaactcttgctattagagtggaatgaaaaaaaagctctagtggccttatataaggtctttttaatcttgtgttaaaatatatctctgcacacctacttataatgaatattcttgaaagattgattcattgaagtgaaatatctggaaggatcacctttttaagcagcttttgaaatgactaatgcccaggtcccacactgcggagattctgatttcattttttggactggggtactagcattggttttaagaaatcagaagatgattctcttgtgcagtggttacagaatgttgcattaatcagtgctctgttactgctgacaaaaaccctacttgaactaatttaagcaaaaagatgaatttatataaaggatatggggtatgggctgggtacggtggctcacacttataatcctagtgctttgggaggttgatgtgagaggatcacttgagctcaggaatttgagaccaatgggggcaacatagtgagaccttctctctcttacaatttaattaaaaaaagaaaggatatgggatatgttgattgaaggataagttgaatatataaaccatgagaaggtcagaaagtggatgaagttttaagttagtagccctggagaccatctttcctgttagcttcattctctttacagatgaacttctttaatgatgtaggtggagaacatggcagctgacagttcctaagggttaagtatttctatgtttaccactgtctgggagttccctctcaaatgcaaacttaaaaaaaatgaggggaggttaattaacgagctagacatgaggaattaggggttgtatacttgattgcaactgggcaaatcaactgggacttggatcccataagaatttcacagtccccgctatgtctatttatggaagagtcagtctgtctctcagaggaaggaagcggtgctgagctaacgataacgagagatctactacaaatcaggcacatgtgaacttaatataaataacaaacataaacggattgtacaaatttgtattccctttatgagagtagtttttccccagctattcatttattcatgatattcatttgatgaatacttactgtggtctgcactctgcctgatactagtttaactgttggatgtgtgggaagggcgaaggaaaatgtaagaaagataataagacgagttaatttttgaaggtgataagtttgtgtcttaaccactcagccattacagcaccttcagaagaatcttgcttccagttatgttcctattgcctgttcaaagcaccttgtccacattatcttcccttttcaaggaatgtgtacatttttttttgtcttttaaaactttttttttttttttgagatggagtctcactctgtcaccaggctggagtgcagtggcgcaatcttggctcactgcaacctgcaactccctggttcaagtgattctcctgcctcagcctcccgagtagctgggactacaggcgtgcaccaccatgcccagctaagttttgtatttttagtagagaagaggtttcaccatgttgaccaggatggcctcgatctcctgacctcgtgatccacctgccttggcctcccaaagtgctgggattacaggcgtgagccaccatgcccggcctaaaactttttttttctttttaagtagagatgaggtctcactatgttgcccaggttggtttcaaactcctgagctcaagtaatcctccagcctcccgaagtgctgggattacaggtgtgagccaccatgcctaacctcacatttgttaaggaatttagaaggcaactttgctaggcacggtgactcacacttgtgatcccatcactgagaaactgaggcaggagtatcacttgagtccacgagttcgagaccagcctgggcaacatagggagaccccatctctacaaaaatatttaaaaattcgctggatgtgatggcacatgacgggttccagccattccagaggctgaagtagaagggacactggagtccaggaggtcaaggctgcagtgagccacagtcatgccactgcactccagcttgagtgacagagtgagaccatgtctcaaaaacataaagaaaggcagcttcaactcatgcactgagccctgaggttactgggaacggcaggtgcaaatatctcatgaggagatagaaagttggcataatttggatgagaaaggctactatggctgcaatatggtgagggaggaggttggagcttcagaagcaggtggaggacagatgtgcatgatttagagaaccaagggaaggaattgggtttttatctgaaatctgatgagaaattatgtttgcaattgatttctcatcagattttactaaaatacagagaatcagtaggaagggtactagctgtactctgatagatttctaagtactgcaaatggtgctaagtgctttgtagctgttctcttatttaatccttagaacaatcctgtgaagaaccttctaaatctgatggtatttatggctgtaagttgctggcaatgttttaatctctttgtataaataaagtcattttagccttacaactactgaatgaaatcagtattctttttacactcatatttcagatgtggaagtggaggctcagagaagttatgtaatttacccaaggttaactgactagtgggtggcaaagccaggatggaaactaggcattctagttttccaagcctgaacactcaaccttcatacttcattgcctctccattccctcatgttgttactaagtcacgtaggtaagtaagtagcgaaaccaagagtggagccaatattttgaccctaaaatccatgcttgcaaatactacttgatactgggctgaacagatttatagatggaaactatgagttccctttgaacatattaaggtgcttatgtcttagcttagacttctatgagtatcttaaggagattctgtgcttataattctaatcttcctaaaaactcttagtaggtattatccccattttatgagtaatgaaaattgaggattagagtttaggtttgctcaggttggaatctaggtcaggaattggcaaactcttttgtaaagattcaattagtaaatattttaggcattgaggactgtacagtttccattgcagctactcaactgctgtgggtcgaaagcagccatagacaatgcataaacaagagagctcggttccaatacagctttctatatggacattgaattttgaatttcaataattttcatatgtgaattattctgattttttccaactatttagcatgtacaattattcttagatgtgagagtacaaaatcagacagcaggctggatttggctggtgggctatgatgtaggtaattggattctaaactttggagtgttttcacaaagctactaataaaaaatcacagaaatataaattacagaagcttcttcataggattaatccaaggattaaataggagaatggctacaaagcacttaagttttattttatttttaattgacaaattatttatttgtgaggtacaatgtaatgttttaatatatgcataccgtactgagccttttttcttttgcatgccactggcacttagaattaaatttgtgagcgggtttcaagttgcgttgtgatgaggctaccagtaagtggcactcttacctcagatttaagagctgtagctggttgcagagtagactaaaatgaagaaactcagaggtactgcttaccctaacttaactctgggtagttagtgtcagaattaaattgatcattagatacgtactttgtctgttgaattagagaaatggttggtgttggaaaacaccccagaatcctagggctctactgtttaatcagctgtatatctcggtttcgtatattaacttcctgctgggcggcgtggctcatgcctgtaatcccagcactttgggaggccgaggcaggcggatcacctggggtcaggagttcatagaccagcctgaccaacatggtgaaatcatgtctctactaaaaatacaaaattagccgggtccatcctggctaatacggtgaaaccccgtctctactaaaaatacaaaaaattagctgggcgtgatggcaggcgcctgtagtcccagctactcgggaggctgaggcaggtgaatggcgtgaacccgggaggcggagcttgcagtgagccgagattgtgccactgcactccagcctgggcgacagagcgagactccgtctaaaaaaaaaaaaaaaaaaaattaaccgggcatggtggcgcatgcctgtaattccagctactcgggaggctgaggcaggagaattgcttgaacccgtgaggcagaggttgcagcgagctgagattgcaccattgtactccagcctggacaacaggagaaaaactacgtctcaaaaaagaaaaaacttccataaaactcaatttccttaacaatggtaaacataatacacatctccgcatagcgtaggtaccatctaacgctgagaatgaaagagaagacccatcctaattcttttagtttttttttattatacttattatattaagttctggggtatatgtgcagaacgtgcaggtttgttgcataggtaacacgtgccgtggagacccatcctaattctttctgaaactcatttgcctcgaagcgacatactcagggcccgctaaacatctgtgcagaagcatctgatctgaaagggcagcaggagtgccctggaggcttaagtggtgcttgattctggggctctgagtcactgctacttgcaggggagactgataacgaaaaaaaaaaaaaaaaaaagaggaaggcacaaataatagcagaaatgaaaggggggacattattatagtaaaaaagtaaaatatggattttttaataagttagaaaatttagacaaaatggacacattcttagaaaattaccaatacagattgaggaataggaaaccaggataattctctaacaattaaaataaatggatcaatagtcaaaatcttctcacaaagaaaacttaaaagttcaaatagctatactggtgaatcttaaagtcagctgattagcaaaagcaaataagcaaacaaaaacacaaaaccaaacaaacaaaaaatagctttgctggcaagtctgccaaacattgccagaaaaaaatacaaaaataaatgaataaataaataaataatgatttcagtcttatacagactctttcagagaatagaaaaacaggaagtatgtcccaactgtttttatgaggctagcacaaaatactagcaaattggacccaataatatgtaaaaataaaaggtaatgtattatggttaatctgtgtttatctcaggaatatagaatagataatatgtcacaacaatataaattgaagaaaaatcacttggtcatctcaaaagatatgcaaaaggcagttataaaatttagcattggccaggcgtggtggctcatgcctgtaatcccagcattttgagaggctgaggcaggtggatcgcttgaggtcaggagaccaagaccagcctggccaacatggcaaaaccgtgtctctactaaaaatacaaaaaaaattagctgggcgtggtagcccacacctgtaatcaatcccacctacttgagaggctgaggcaggagaattggatgaacccaggtagcggaggttgcagtgagccaagatcgtgccactgtactccagcctgggtgacagagtgacactctgtctcaaaaaataaataaataaataaataaattagcgtctatccatgaaaaagaaaaaacctttagaacattaggaatagaagaagacttccttaatataataaatagtagctccataaaacctatagcatgtattatactcaagatgaaaacctgtaaatttcctttgagagctggagtaaaacaaggatgcctgctatcaaaatgtctattaaatacgtactagatttcctaaccagcatggtaaggaaaaaaaaaagtagtattgaaaaggaaggaacaaaactgtcgttatttgcagaaaatggttgttaacatagaaaattcagtgtaacccacagataacttattaatgagtttactgattgctgaggaaaaaatattcaaaaatcaattttattctggtataccagcaacaaacaataacttaaaaatgagatactcagccaggagtggtggcttatatctgtaatctcagcactttgggaggctgaggcaggagaatcgcttgaacctggaaggtggaggttgaggtgagccgagattgcaacattgcactccagcctgggcaacaagagcgaaactccgtctcaaaaaaaaaagtttttttcctctacttcctcaccagcatttatgttttctttttttcgtaattgctgtcttgaggtgagatgatatctcattatagttttgctttgcatttctttttcttttttttttttttgagatggagtttccctcttgttgcctaggctggagtgcaatggtgtgacctcagctaactgcaacctctgcctctgggattatagatgtgtgccaccacacctgattaattttgtatttttagtagagatgggatttcaccatattggtcaggctggtctcaaattcctgacctcaagtgatccacctgcactggcctcccaaagtgctgggattataggcatgagccactgagcctggactgcatttctctgataattagtgttgttgagcatttttttcatatacttgttggccatctttatgtattttcttttgagaaatgtttgtacagatcatttgttcattgtaaaatcagattgtttattttttgctattgagatgtttgagttccttgtgtattctggacatttatcctccactagatgaatgaatagcttgcaaacactttctcccattctgtaggttgtcttttcattcttttgattgtttcctttgatgcgcagtttttgtttgtttgtttgttttttgagacagagtctcactctgtcacccaggctggagtgcagtggcacgatcttggttcactgcaacctccgcctcccaggttcaagtgattctcccacctcagcctcctgagtagctgggattacaggctcatgccatgatgtctggctaatttttgtgtttttagtagagacagggttttgccatattggccaggcttgtctcgaactcctgacctcaggtgatccacgccccctggcctcccaaattgctgggattacaggcatgagccaccatgcctggccctgatgtacagttttttagtttgatataatcccatttgtttatttttgcttttgttgcctgtgtggttgaggttttattcataaaatattttctcagaccaacgccttgaagcaattcccttatgttttcttctagtagttttatagtttggggtcttatatctaggtctttgatctattttgagtttatttttgtatagggttataggtgagggtctagtttcattcttctacatatggatatccagttttcccagcaccatttattgaagagactatcctttccccaatgagtattgtcaaaaatcagttggctgtggatacatggatttatttctgggttctccattatcttccactggtctatgtgtctgtttttgacacacagtatcatgctgttttggttacaaccgctttgtaatatgttttgaagtctggtagtgtgatacccccagctttgttctttttgttcagagggtcttttgtggttctatgcaaattttaagatttttaaacaatttctcagccaggcgtggtggctcatgccggtaatcccagcactttgggaggccgaggcaggtggatcacaaggtcaggagattgacaccatcctggctgacatggtgaaaccccgtctccactgaaaatgcaaaaaaattagctgggtgtggtggtgcgcacctgtagtcccagctgcttgggaggctgaggcaggagaatggcctgaacctgggaggcagaggttgcagtgggccaagatcgtgtcactgcactccagcctgggcgacagagcgagactccgtctcaagaaaaaaaaaaaaaaaaaaaaaaacaatttctctgaagaatgtcattggtattttggtaggcattgcattaaatctgtaggttgctttgggtagtatgatcattttaacaatataaattcttctgatccattagcatgggatgcctttacatttattttatccttttcactttttttcatcaatgttttgtagttttccttgtagaggtctttcatatccttggttaaatttattcctaggtatgtttttgtaactactgtaagtgggattgactttaaaatttttttttgatgagtttgttgtttttatataaaaatgtgaccgatttttgtatgttgatttgtgtcctgcaactttactgaatgtgtttagcactatgttgagtaaaagggcatccttgtttttttccaattattagaggaaaaactttcagcttttccccattcagtatgatcttagctgtgggtttgtcatgtatagcctttattgtgttgaagtacttttatttatttttcttttgagacaaggtcttgctctgttggccaggctggagtgcaagggcacaatcttggctcactacaacctcctactcctacgctcaggtaatcctaccacctcagcctcctgagtagtgggactacaggcatgggccatcgtgctcacctaacttttagaacactttttgtacagacggggtttcaccatgttgtccaggctggtctcaaactcctggactcacacgatccaccctcccaaagtgctgagattacaggcatgagccaccatgcccagcctggtacttttctactatacctaattatttagagtttttgttatgagggtgtgttgaattttaccagatgctttttctgcttctattagatgatcgtgtggtttttgtccttcattcttttaatgtaatatattacatttattgatttatgtatgttgaaccagccttgcatccctggggtaaatcccacttgatcatggtgtattatctttttgatgtattttggattcactgtgctagtattttattgaggattttagatctgtgttcatcaggaatattggcctgtggttttctttttttgttgtgtccttgtctagttttgttattagttatgctagcctcatataatgagttaggaagaattccctctgctttaatttttaggaatagtttgagaagaattggtttgaattatcatttaaaggtttctgtagaattcagtggtaaagccctctgatcctggacttttctttgttgggagaattcccttccttcgccttcccctccccttcccctcccctcccctcccctcccctccccccttcctctgtctttctttttccctttcttctgttttttttaaagaaatgggcttttgcttagttggcccaggctgctcttgaacttttggcttcaagcaatgttcccatcttgatctcccaaagtgatagaattacagatgtgagccaccatgcccagtctgttgggataatttttcttactgattcaatctgttaatggtctgtttgggttttctgtttcttcttggttcaatattgataggttgtatatgtccagaaatttatccatttcctctaggtttttgtctctttatacagcttttgacttgaagtctgttttgtctggtataaggatagctactcctgcttgcctttgctttcaggagcatatagttgttaataatagtctctaatgatccttttaaaaaattttggccgggtgcagtggcacatgcctgtaatctcagcactttggaaggctgaggtgggtggatcacttgaggtcaggagttcaagaccagcctggccaatatggtgaaaccccatctctatgaaaaatacaaaaattagccaggcatggtggcaggcacctgaaatcccaactacttgggaggctgagacaggagaatcacttgaacctgggagggggaggttgcagtgaaccgagatcgtgccactgcatgccagccagggaaacaaagtgagactccatctcaaaacaacaacaacaaaatattttttttatttactaatttttttgtagagatagagtctcactctgttgcccaggctggaggctgttctcaaactcctagctacaagcgattgtcttacctcagcctcccaaagtgttgggattaccagtgtgagccaccacacctggctctaatgatcctttgtatttctgtggtattcattgtgacatctcgttttttatttctgattttatttgggtcttctctctttttttcttagtcggtctagctaatggttcattaagtttgtttatcttttcaaaaaatcaccttttgttttattaatcttttgtatttttatttttgctctggtatttattatgtctttccttctaattttggatttggtttgttcttcctttagtagttctttgagatatgtcatgaagttgtttattgaaatccttctttttttcttttctctctagaatttattttgtaggatagcatatttgaaatctttctagtttaattttttttaatttttattattgctgttttttttgaggcaggttcttactctgtcaggttggagtcaatcatggctgcattgttggcacaatcatggctcattgcaaccttaaattcctgggctcaagggatccttccacctcagcctcctgactagcagtactacaggtatgcaccaccatgcccagctaataaagttttttatttttgtagagatagggtctcactatgttgcccaagctggtcttgaactcctggcctcaagcaattctcccacctcggcctcccagcgtgctaggattacagatatgtgccaccatgcctggccctctttctagtttttcaatgtaggcatttattgctataaacttgcctcttaatggaggttgcagtgagccgagatcacaccactgcacttcagccttggtgacagaatggagactctgtttcaaaaaagcccccaaaaaacaacaacaaaaaacttgtctcttaatactgctttacctgtgttccacaggttttgatgtgttgtgtttctattttcatttgtttcaaggaaacaaagagagatatatatatacatatatatatatatatttttttttgagacagagtctcgctctgttgcccaggctggaatgcagtggcatgatctcaactcacctcaacctctgcctccagggttcaagcgattctcctgcctcagccccccaagtagctgggattacaggcatgcactaccatgcctggctaaattttttgtatttctagtagagatggagtttcaccatgttggtcaggctggtctcaaactcctgaccttaggtgatctgcctgcctcagcctcccaaagtgctgggattacaggcatgagctaccgtgcccggccaagagtgatatatcttttatatatatcttttgttccttaatttctctcttaatttgttttacttgctctttaatttgtttcatttgttttgcaatttcattcttggccaggtgcggtggctcatgctgtaatcccagcacttggggaggatgaggcaagagatgcttgagcccaggagtttgagaccagccaaggtaacatagcaagaccctgtctctacaaaaaatagccaagcgtggtggtgcacacctgtatttccaggtactcagtgggctgaggcaggaggatcccctgagcctgggaggtaaaggctgcagtgagccatgatcacaccactgcactcctgcctggaagacagatggagacttggtcataaataaataaataaataaataaattttaaaaattttattcttaatttcttcttcatgcattagtcattcaggagcgtgtcttttagtttccctctatttgtatcattttgaatattcctctttttattgatttctagttttattccatgtggtcaggtaagatacttgatatgattttgattttgaaatttttttttagccaggtgtggtggctcatgcctgtaatcccagcactttgggagcccgaggcaggtagataaccttaggtcaggagttcgagaccagcctggctaacatggcgaaaacccatctctactaaaaataataataataaaaaaagtagctgggtgtggtggcaggtgcctgtaatcccagctacttgggaggctgaggcagaagaattgcttggacctgggagtcagaggttgcagtaaactgaatcttgccattgcactccagcctggctgacagtcctggagaatgttccatgtgctgatgagaagaatgtgtattctgcagctatctagtgaaatgttctgtaaatgtctgttaggtccatttggtttatggtgcaatttatatccagtgttcctttgttgattttctttctagaaatctgtccaatgctgagagtatagtgtcgaagtccccaactgctttgtattgggatcgatctttccctttagatctgacaatatttcctttgtatatctgggtgcatggtgttgggtgcatatatatttacaattgtttgattttcttgatgaattgatccatttattattatataatgtccttctttgtctctttatacagctttgacttcaagtctgttttgtctgatataagtatagctactctctcttgctttggctttccaaatcagaaatccttaacttctctcttactatttttgcagttgagtggttttctctagtgataagatattattcctttctatttcttctttgggtattggctctaccaatgagttttatatttttgcttttcaaaagcaagcaggagtaaaaaagaaaaagaatatctttttccatgagcaaaaatctttgtccaaaagcaaaacaatatctttttcaatcctttcatttccaatctatgtctttacatataagatgagtttcttgtaggcagcctatagttgggccttgttttattatccattcagccagtctttatcttttaaatatggaatttaattagtttacattcaagattattattgatagatgagaacttattcctgccattttattgactttttttgttttgtgtatcttttgttccttaatttctctcttactatttttgcagttgagtggttttctctagtgataaggtattattcctttctatttcttctttgggtattggctctaccaatgagttttatatttttgtatctttccatgatggtggttcttgtctttcacttccagatgtaagactcccatgaacatttcttataaggctcatctagtggtgatgagttccctcaatttttgcttgtctgtgaaagattgtatttctttttcatttctgaatgatagctttgctgggtatgaagtttttggctggcaggctttttttttttttctttcaaatccttaaatcattctctcctggcccataaagtttctgctgagaaattcactgttagtcttatagagattcccttacatatgacccaacacttctctcttgctgtttttagaattctgtatttatctttgacttttgataatttgacataatgtgccttggagaggacctgtttgaatctaatgtatttggggttctttgagcttcctagatctggatgtccatctttctcccaagacttggaatgtttcattaaatatgtttttttaatataacttttcccttttcttctgcttctggaatgcttgtaatgcaaatatttgtttgcttagtgatggctcataaaccctgtaaactttctattctttctttcttcttcttttttttttttctgtctgcctatattatttcaaaagacctgtcttcaagttcagggatttttttttttttcctttggctcagtccattctattgtcagaactctcaattgtattttttattttattcattgaattctttagctttagaatttctatttggttcttttttaaatgatatatatctgttgaatttctcattcaaattatgaactgttttcctgatttcattgaattgtatatctgtattctcttgtatctcactgagttctattagaattattattttgaattctttttctggcctttttaatatttccttagtattggggtctgttattggagaactattgtgttctttgggaagagacatgttttcttgcttttccttacaatgatttctatgcaagtcagtgaaacatttgcctcttccaattctatgcagtaggtttagtaggcaatgacttatttgcatagatggtcttggagtgttggttcaatggggtgcactggctttggttctaggtggatgcagtagtgcagtgtccatgtagtttcttcagctgtaatccatgttagtgatgtttgcatgtgtctcagtggcataggctaagagagtttgtggcagtggtggtgcagctctgctggaagtgggctttcaggttatttcaggttatttctcaggtcagtgtgcctacatgcccatggtgggttggccaacttggggtctcactcatcagggttgggaccacagagcttttactttaactaagggcgtcagtgtgctgtttcatggctagcctgaaggccgatcatccaggggtggcccattgtgctgtttcttgagtccagtacactggttgctttgctggcctgggggtgtgtgtctcctggggatggtccttggggctgttttttagttctgagtagtgagtaaaggttatttgggctggcctggggggtgtctccttagggtggtccacagggctgtttcttaggcctgggatgtggccatatggctttttggctggccatatggtatggtaaaggtggaccacagggctatttctcaggcccaggatggggtgcatagctgctcagctggcctgggtgtgtgtctggtgggggtggaccatggggctgtttctcaggcctaggatgtgaggggacacacagttgcttggccggcctggaggtgtgtctgccaggagcagcctgcaaggctatttcttaggccctggacatgggcacatagcagcttgatcagcctgggagcatgccagacaggagtggcctgtggggctgtttccctggcccaagctgctcagttggcctgggggcttgactgccaggaatggctcattgggctgttttctaagcctgagacatggtcacacagctgcttgttctgcctgggtatgcgcctgccatgggcagtcaacagggctatttctgaggtcctgatttggggtgcagggcctttgggcaggctagaggtatgtctgcaattgtaggagggggtgctagatgccacagggctgtttttcaggctttggatgcaggcatgtagccaccctgctagcttacaaatgtgtcagctgttggatggctcagggacctctcttgctcaggggagggcctacagtagtttggctggctttagggtgggtttgccgggggcaggactgccagactatttttctagctgaatatgtgagcagcaggggttggtttccctgctgggcaaggccagagtcagagctaatcctgggcccagcctctgcagagttggggttgtgacatttagtcacctgtgtgggcttggcagaatgaagatggagccctagctggagagatgtagtggctactggcccgcagaggaggatgcactccagatgtggctctggtctcaagatggcactatgctgcaccagtttggctcacaccaggttgggggaccgagtaggggtggggagtgcataccttgtatcccaatccagagcacacagctgcattaattccctacacctctcctaactgggctcagggcttgtgaggaccttgtaggattctcctgtagtaaggactgtaggtatttttggtggcagtgggggctgctggggttcttctacttactgttgccttgcaagggaaagtctcttctgtttcttggccagtctgggctggggagatgggggctgcagaagctgggtgcacccatagtgtcctcctgggtttctagtcatcacaggtgcctctctactttcccactgtactctagtgctgtccctttgacatctggtcaaatctcagctattatttattgccttggtcctttcttttgggggagaggagtgccaggcatctctagtcagtcatcttgcactatgtgatttctgaaataggatagagatgtcattacaaagtagtgggaaaatgatgaaatttccaataaatggggctgggataaaacacgcacaaataaattccaggaagattaaaggcttaagcatggaaggcaaaattataaagatttaagttaatgtagtcaaatatttttataatctctggataagaggagaatttcttaaaaagtactaatactaatgtactactcagaaaaggaaagattagtcaactttactatattaaaattaaggaagttcaagaaaacataccgtatatttccacgtggatagtgtaaaaaggaaaaaaaaagaaagaaaaaaaagaaaacacagtgtaaagtgaaaagcccagtgtgggagaaaatatttgcaatggatttaaacagaagataactagtaccagaatatataaattatttgcataataagaaaaatatagaaactcgatgggaaaatgggcaaaagagttgaataagcatttgtccaaaaaagtccaaatagttcctaatcaaaataaaggatcctgactgggcacggtggctcatgcctgtaatcccagcactttgggaggccaaggtgagtggatcacctgaggtcagaagttcgagaccaggctagccaacatggcgaaaccctgtctctactaaaaatacaaaaattggccaggtgtagtggtgggtgcctatagtctcagctacttgggaggctgaggcaggagaatcgcttgaacccaggaggcagaggttgcaataagccaagattacaccactgtactccaacctgggtgacaaagcaagactctgtctcaaaaagaaaaaaaaaatggccagacacagtggctcacacctgtaatcccagcactttgggaggccgaggtgggtggatcacctgaggtcaggagttcgagacctacctgtccagcatggtgaaaccacgtctctagtaaaaatataaaaaccagctggatgtggtggcacacccctgtaatcccagctacttgggaggctgaggcaagagaattgcttgaatctgggaggtggaggttgcagtgagccgagatagtgccactgcactccagcctagacagaagagtaagactctgtttcaaaaaacaaaaaatgaaaaaagttaaaaaaataaaaataagaaacaaaagattcttaacttattaataactatgaagattcaaattaaaaccacagtgagatgttattatacctttaccaatttggcaacaaacaaacaaaaagacaaaaaagccaactttgataataccaagcgttgctgagaatgtaggataaagaaattgctgtatactgctgctgggagtcttagaaactaattaggcattctctagtaaggttcaagatgtttataccttctgacacagtaattctgctctagaaatgtagtccagagaagcttctctatttgtgtaccaggattcatctatgagaatgtgcagaatggcattgtttatgataactaaacaaaagataaagtagaatgaacgtggccgggtgtggtggctcatgcctgtaatcccagcactttggaaggccgaggtgggcagatcacctgaggtcaagagtttgataccaactggccaacatggtgaaaccctatctctaccaaaaatacaaaaattagcttgtcgtggtagcacacacctgtaatcccagctactcgggaggctaaggcaggagaatcgcttgaacctgggaggtagaggtagtactgaggtgagacttagccactgcactgcagcctgggagacagagcgcaagattctgtctctaaataaataaataaataaattagaatgaacaaagaatatgaggtatattcattcccagaaaaactgtttagtaaaaatacataaacaacctacagggatacaaacagcatgaatggatttcacaaatatgctattgacttaaataaaacacaaaagactacagactatatgattttattcacataatgttcaaacccaaggaaaactatacatataattttgggatacacacatagctggtaaagctataaaaaagactataattgcaaaattcagggtgatttcctgtaggagagagggaagatttttataaataatttttttttttttgtagagatgagatctcactctgttgtccaggtcttgaactcctggcctcaagcagtcctcccacttggcttcccaaagtgctgagattacaggtgtgagccattgcacccagctactttgttttggagagacagtcttgccctgttgcctaggctggccttgaactcctggcttcaagcaatcctcctacctcagcctcccaggcatgagccactgtgccaagctgggaagattttgtgatcaggaagaagtacatggtcttggcatggggaatagaagagacttatggagactagcagtgtttttttttttttttttggagtcctagttacttgagtttttgcttcaaagtatttataaactataaatttatgtttatataacgtcctatattctgttatatttccattaaacagtagtttgaccttaaagccatattgtgacatcattacatatccattgttttggaaaaaatgaaatagcctgatggtactgatattggcagcagcatagaacatcaaaagagttctcactgtcggtgagaatgtgagcttttgaaagctgctcaacattcccctgtaaggtggatgcctttgagaagagaaaaatgagaatgtgggtcagagacaatgcgggggaaatagtaaagtgaaaaaaaaaaacaaaaacaaaaacaagcaaaggacctcaagggactgacgatgagtacgtccatgtattgaagactatgataagcttaactatctgaacccggggtaaaaggctaataaccagcaaacagaaagataagggcgatgccacactggaaaccatgctcagaatgagagaccactcatgctgctcccccaccagtggggcctctgtaggaggttgtgggaccttcactttcaagatgagactgcagataagatttcccacagtaactcacatagctcagctgaattttttgggtgaggaaccccttttcattcttttcattttcatcattgggagcctagctgagctattaagggtgtagtatttctatacccctctccatttcctctactcgaaagacatttagttactcaggcaggagcagcatcaatttcccttagaagttacttaatggataaggctgggtgcggtggctcacacctgtaatcccagcactttgggaggctgaggtgggaggattgcttgaatcctggagctcaagatcagcttgggcaacaaagtgagaccccttctgtacaaaaataaaaataacaaacagggtgtggtagctcatgcctgtggtcccagctgctcaggaggctgaatagggaagataagtatttatcttgctccatgacactgccagtattactaccatcagactttttaattttttataaaacaatggatatgtagatgtcacattatggctttaaggttaaactattgtttaatggaaaaatataacagaataaagaacattacatagcataaatttatagtttgtaagtacttataaactgtaagtattgagcttaagcccaggagtttgaggctacagtaagtaatgcttgcactactgcacttcagcatgggtgacagagagagactctgtcactaaaataatttttaaaaaattacttgctggataaatgttgtgatagctcagaatgctgacttgtccttcctgggccactgtattctatattggaaggggctttaagctggccttgcactcacttgagtgtgacctcaagcttaatctctctggcatccattcatggttactaaataaaaaactctctataaacacaggagctctttatcatctcttcagggttgtgaagagcatgtccttgtaacttaaaaaacgctgcattagtaaaatgtcatttttaggtaagaaaaaaaccatatatacttggaggagaaagtttctttccccatctcctgcctcttccctttttgtgtaaggggtctctagtggatgtttgtttttgttgtccaacttctgtctctcctcctgaaatgcactgcagtttttatttaggggaatcactcttctctcatttcagaacatgaagtgtggggaggtgacccaggcctaagcctattaatgctttttgaccctttggggacagtgatttttcaggaatgagtctgtgactcagacccagacttgggcctttgaaagagtataaatcccttaaccaataggggagtgattcacacagatgtgtgctctgtgggtgcaagagaggggcactgtgtagaatctgagaaaagagacagccctggaggcaactgaatgttaacatcagtcagaaactaggttcttggtgacctctctgagagccactgaatcaagccttgcctgaacctggatttttccataatgtcagttaataaattcactttatagttgaagccagcttgagttgggttttctctcattggcagcataataatgtatggagtctttggtaagagagttttgttggcatatgggtgcatcaaaatcaatataagaacacgccatagtcatgctgtgagaggtagtcaatagcaaaggggaaatttatttatctttgtaaacacgttattctgaagaccctgaaaaacctgaagtttcttccaggtatgcccaggatcttggtggtctgacgtagccctcttagtgctgggacctggtgaggagggcaaaagtgttgaaagttaacttaagctgggcccagtagctcacacctataatcccagcactttgggaggctgagatgggcagatcacttgaacccaggagttcaagaccagcctgggcaacacagtgagattcccatctctacaaaaaaaaaaaaaaaaagaatctgaaagaagactcattaagttgaaagtggctatttcatggtactgagattatgacaacatttgttttatttatttattttttaattcgtatttttcaaaattttctgcagtgaatatgatttatataattttttaaaaaggtttttaaagtactgtacaaacacacaaggtgacagtacccttcatgggaactcacgtggctagttacaatctggaagtcagaacatctggctcacaattgcttaatttaacctcttacccttaatattcaacctgtttcttcctttgcaaaattttgaactaatctcagttaggtctatgtaacctgagatgtgatttaaccaagttaattatcaactcaatttaagtacatatagggtccccaccttaagtggtcattgtggttactccccagtatctactccactgcacttcccaattgtgtagggaagattctagttggactgagattttccggatgctgccatctaaataacccatattatcctgtctcctgatctggctctaacttccattttatgaacttttgcataattagagttacactctatgcacactgtgcctggcccagtgagttcatctaatggagtctatgtagctgtaataaaaaaatgaggacattcggccaggcgcggtggctcacgcctgtaatcccagcactttgggaggccaaggcgggcggatcatgaggtcagaagatcgagaccatcctggctaacacgatgaaacccagtctctactaaaaatacaaaaaactagccgggcgtggtggcaggcacctgtagtcccagctactcgggaggctgaggctggagaatggcatgaatctgggaggcagagcttgcagtgagccgagatcgcgccattgcactccagcctggggacagagcgaaaaaataaaaaagaatgaggacattctacatgagctgaaacaaaagattttaggctgggcgcggttgctcatgcctgtaatcccagaactttgggaggccgaggtagttggatcacttaaggtcaggagttcgagaccagcctggtcaacatggtgaaaccttgtccctactaaaaatacaaaaaatttgctgggcatggtggctcgtgtttgtaatcccagctactcaggaggctgaggcaggagaactgcttgaggcagagtttacagtgaaccgagtttgcaccattgtgctctgacctgggtgatggagcaagacttcgtctcaaaaaaaaaaaaaaaaaatttaaatacatttaaaaaagaaggttgcatgctgtggagcaaccagacaattgtgatgaaatgtgaagcacaaggcaccagctgtgacgtgtttttgccaagaagtcaaaccacgttccaactaaacctctagagcaaactttcattttcagcaaattcgaagaaaagaggaataatgtaaatgaccccacagggaaacagacaaaccctgaatgtggagcatttcacaggacaaaacctggtttgtataactggtttgtataaccaatggcaagggggtggagggcagactgtcttcgattaaaagagacttgagggacatagcaaacaaatgcaatgctgggacttgtttggatctggatttaaacaaactgattgaaaaaagacatttttgagataatcagggagatttggctatagactgaagtactaggtgcttctaaggcatcattaaatttgttatgtgcgataatgatattatgattatattagcagaatgtccatttttgtttgttggttggtttgttttgagacgaaattttgcgcttgttgtccaggctggaatgcagtggcacaatctcggctcactgcaacctctgcctccctggttcaagcgattctcctgcctcagcctcccaagtagctgcgattacaggcacccaccaccatgcctggctaattttttgtatttttagtagagatggggttttcaccgtgttagccaggctggtctcgatcttctgacctcaggtgatccacctgcctcggcctcccaaagtgctgggattacagccgtgagtcaccacacccggcttcagaatgcccattttttaaaaaggaagattgcacactaaagtacgtaaaagtgaaatagcacgatgtctgggattcgtttaaaaatatttcagcagactcggcatggtggctcacacctgtaatcccagcactttgggaggctgaggcaggaggatcccttgagcccaggagtttgagaccagcctgggtaacctagtgagacccttgtctctaaaaataaattaaaacaaatttaaaaaaaaataaagggccaggtgcggcggctcctgcctgtaatcagcaatttggaatgctgggacgggcagatcacctgaggtcaggaatttgagaccagcctggccaacatggtaaaaccctgtctctactaaaaatacaaaaattaaccgggcatggtgacaggtgcctataatcccagctacttgggaggctgaggggaggcagaggttgcagtaagccgagatagcaccattgcactccagcctgagagacaagagtgaaactccatctcaaaaaataaataaataaataaataaaaataaaataaataaaataaataaaattttaaaaataaatatttcagcaaataacaataggaaaatgcaagtttcaaaaaaggtatatggtattttgtctaagaaaagagagtatgagaatatatattttattttcttgggtttgcattgggagtatacacaagaaacttatgaaaatgaaaacctggtcgggtgcagtggctcaagcctgtaatcccagcactttgggaggctgaagtgggcagattgcaaggtcaggagttcgagatcagcctgaccaacatggtgaaaacccatctctactaaaaatacaaaaattagctgggcacggtggcaggtgcctataatctcagctactcaggaggctgaggcaggagaatcgcttgaacttgggagtcgcaggttgcagtgagctgagatcgtgacgctgcactccagcctgggtgacagggcgagactctatgtcaaaaaaaaaaaaaaaagaaaacctatagcataggtgggagaaatgggggtaaacatggaaggatgggatcatgggatcaaggcttcttgagctttttatattccttttactttttaactatttgaatgtattatctaaagtttttaagaaaataaacaggaaataaaagttaagtgaacatgaaaatgttcagaaccagctccctacacacacaagagagttaaaggagatctctagaccccttcatatctagactttaatgaactaataaattgttaatgttttataaagctggtctcagtagcaattctagaaatgagcagaaaacaaagtggtaaaaacaaaacatcgctgggcacggtggctcatgcctgtaatcccagcattttgagaggccgaggcaggtagatcacctgaggtcagaagttcgagaccagcctgaccaatatggtgaaaccctgtctctactaaaaatacaaaaattagccgggcatggtggtgtgcgctcttagtcccagctacttgggaggctgagacaggagaattgcttgaacacaggaggcggagtttgcagtgagccgagatcgcgccactgcactccagcctgggcgacagagcaagactctgtctcaaaacaaaaacaaaaacaaaacaaaacaaaacacagaaactgagggctgatgcttttaagagccttgaccaagttcatgctgctaataaaaataaccccagaccccatatttaatctattctaccttatgcttgaatatggtcatttgctggttattagttatgtgggaagacaaacattaaattaataaaacataatttatagtggaaaaaatctgtcacttagggggcttatgctctcattctaaatcaccaatcatgtgcctttcggcaaatcaattttctgaagtctcagattccttgtctataaaatgagaaattggccgggcgcggtggctatgcctgtaaacccagcactttgggaggccgaggcgggtggatcgcctgaggtcgggagttcaagaacagcctgaccaacatggagaaaccccgtctctactaaaaatacaaaattatccggggtggtggcacatgcctgtaatcccagctactcaggaggctgaggcaggagaatcacttgaacccgggaggcagaggttgcagtgagccgagatcatgccattgcactccagcctggacaaaaagagcgaaactccatctcaaaaaaataaataaataaataaaaataaaataaaataaaatgagaaattgattatgatctcttgagtaactgccactctgcaatgctatgggaagtacacacattgaacaagttgcatatttatattaaatgccatttgtttatattgaacaaatttggtatttatacccaatgcatttaatggcataaatgtgaatgacaaagaaactcaggaaggcaagacatagaggaaggccactggcttttcagaaaccactcagcctgtgagctcacttcccttttataaaaaaaagcttttgcttttaaggtgtccagataaaggtcacactcccagagctgaggctacgtgagtctaagctgttacctcagttatgggagtgtggctggaactcaacactttctgatcattcatggccaaagcctcttggggtaggggagagggggcaccttgtatgcatgagatgaaacaggataccaagatccccttaattctactcagagtagaggaaaggaactgttgccaaaaggaacatgttttttctaaattgaagagttggaagtttgaaggtcaaagtcagaatattagaacagagagccatgcatgtattatctaatacaattttctcattttataaattatataaatgaggaaactgagactcagcaaggttataggatacaacctaatctgtgttagaagaaggatgagagtgaggcttatgctgggagttgctatggtctgtaaatgtgaggataaggaacatgccattcactgttgtgttccacttcctagtccagtgcctggtgcaaagtaggggcttcataaactttggtcacaggcctgatgaagggactgatttgagtgaatgaatccatggatgatgcatctgtactttgtgttccccgctagtcagatggactcggacatgctccatttcaggtgtatctcatctgagacattattagagggttctctttatgtgccatgactgagtagcgttttcttccactgaagtattgcttcctctttgttgatatcacaggacagacatcagaacacttacaggatgtgtgtagtgtggcatgacagagaactttggtttcctttaatgtgactgtagacctggcagtgttactataagaatcactggcaatcagacacccgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaagtaagtctcggcatggatatttacaatgacataatggtgctgtacagaggacaagggagagatacaaggatggccattgtaatgttgtttgtaatagcaaaagcttggaagcaacctaaatgtccgtcgatgagggagtgggtaaataaatgtgatataatcatgatacaaacagtgagtctgacaaaacatactggtatagaatgtctcaaaattataatgttattaaagaaaaaaagttgcagaataatgcatatattaggcttctatttgtgtgtgtgcattgtcggggagcaggaatatgtggacatttcttaatgtataagctctaggaaaaggaaccagggaatggtaaagagttgggagagtaaggcatatttttcttttcttttctttcctttttttctttttcttttctttctttttctttttttcaattgagatggagtctcactctattgcccaggctggagtgcagtggtggcaccattttggctcactgcaatctccgcctcccaggttcaagtgattctcctgccttaggcaccggagtagctgggatcacaggcgtgcaccaccacacccagctaatgtttttaatttttagtagagacagggttttaccatgttggtcaggctggtctcaaactcatggcctcaagtaatctgcccatctcagcctcccaaagtgctgggattacaggcgtgacccaccgtgtgcccggccactttttcatacacctttgatagtgtttgaatgtttttactgtagttgagtcatatgaaaacaaaggggacataaaaaatcaaaagaaaatatataatgaatgttttatcatatataaaatatatatagtttttttttaatggaattaactttgtcatttgaggaaaatgaggagacaagtctcaatcattttaggagatttgtttgccaaagttaaggaggcacctgagagacaggtctatgactttctcagaagatgattttgagggctccagatttaaaggggacaaggtgggatattgagaagcactcagttttcacataaaaaaggggggggggcagaggaaaaatatggggaatctgcattttacattagataacacagacaaaatggggtagggaaacaatcagataacgcatttgtgtctggccagggtgaccgcaccggtaaagatgagctatcaatctgcattgcacagctgaccaggaatttccttgtgggcaaaatatgggggaggtatgcagcctttcatcctgtagccatcttattcaggaatcaaaaagaggaggcaggtttctgtgacccagttcccagcttgacttttccctttgactgaatgagtttggggtcccaaaatttaatttcttttcacaactttaaaccttaaaacaactccatgagtagtttcaaatacacaagatccttatttaaccaagggtttgtatttactgtgcacttgctgcttaaaaagagaagtatgttaggagagcctccctttgttgatgaacaagcaagtagcccagatgggcgggccgtttcctggctgaccatgactaattttctgattgtctgtttccatcagccctgttctcccgtgttcacagaattgggccacaattctctcctagggcagtgtttctgaaagtgaggttctgagaccagccacttcagcaacacttgagaacttgttagaaataaaagttctcaggctctaccacaggccaactgagtcaggaactctagcagttgagcccagcaatctgtgttttcgcaaggcttcccagtgattctgatggccttcacatctgagaagcattgtcacagcgaatcatcctccaaacaggactgcagcagtagcttcctctttattctgtaagacatggcttgcagttttcctgaaatggagtaacctcactcaccgcttgagtcttggctctccttctctctctatgcagggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgccatctttatgaataagatttatctgtggatcatattaaaggtactgattgttctcatctctgacttccctaattatagccctggaggagggccactaagacctaaagtttaacaggccccattggtgatgctcagtgatatttaacaccttctctctgttttaaaactcatgggtgtgcctgggcgtggtggctcacacctctaatcccagcactttgggaggctgaggccggtggatcatgaggtcaggaattcgagaccagcctggccaacatagtaaaaccttgtctccactaaaaatacaaaaaattagccaggcatggttacgggagcctgtaattctagctacttggggggctgaagcaggagaatcacttgaacctgggagtcggaggttgtggtaagccaagatctcgccattgtactccagcctggctgacaagggtgaaactctgtctcaaaaaaaaaaaaagaaaagaaaagaaaagccaaaactcatgggtggaatgtatagatgggatgaaaggcctagatcagtggttagagaccctgactctcattagaatcacatagagagtttttaaacaactatagatgccaggtacagtggctcatgcctataatcccaatactttgagaggctgaagcaggaggatcacttgaggtcaggagtttgagaccagcctggctgatatactgtggctacccctatctcttaaaaagcagaagaaaaaaccccactaccaaaaaccagagaaatcagcatctctggaggtagatgctaagcattagctttttacagagtctacctcaggtgattttaccccaggtgcagccagcatggtgaactgctgggttatgcatgtatgctgggaggtgaacagacttaagatttctataacatgtactttgtgcccagatagagctgagtttacatctaactctgcctcttactagctatataatcctgagcaagttattgaaacgctgtgagctcctcttctataaaatatgacaataataccgatcatgtgggccattagaaggattttatgtgataatattttattaaaagcctttataggttgggcatcccaaatctgaaaatctaaaacctaaaatgcaccaaaatctgaaatgctctaaaatccaaaactttttggacactgacatgacattcaaaggaaaagctcattggaacataagtataggcagatactaaaaaataaaaaaataaaaacatggctgggcgtggtggctcatgcctataatcccagcactttgggaggctgaggtgggtggatcacctgaggtcgggagttcgagaccagtctgaccaacatggagaaaccctgtctctactaaaaatacaaaattagccaggtgtggttgcacatgcctgtagtcccagctactcaggagtctgaggcaggagaattgcttgaacccaggaggcggaggttgcagtgagccgagattatgccattgcactccagcctgggcaacaagagcaaaactccgtttcaaaaaaaaaaaaaaaattcaaaatctaaaacatttctgatctcaagcatttcagataaggagtcttcaacctgtaattatggccatttgtgtggctgttagttccgctattaataatcagaatgtaacaatgatgactgcatcttacctttgtcaaacatttgaagtgtgtgatacactctcaacgtaggtatccaggcaggtttggaagctgagggtgtatttctctttcttaattgtctttcgcatttttttcttgctgcccctttgattccctgaaataagctttctgctctcagataattttcatccttatgttgtttctgacattagccaaggtcacccctgcaccgactcggcagagagacttcactgcagcctttccaaggatgcttaccaccaggggacatggcccaggtaactatgcatggggcagaggagggaatctttagctggctctgaattcagaggattttgaatgtgggaaaaaaagatgagaaaacagtgtgagagtggaggcatggatatctccgtgcacctgaaatagtgtttgaaaagagggaagtggtggagacacacctgtgctgggaaaaagatggtctggcttaggattattttacttaaattttaactggaaaaaagttaataaactttatagtctttaattttacttaaatattaactaaattgaatgaaatgctcattactttgtccacattttctgactgaccctggactggtgatgtttggtcatgaactggtgtcagtctgtgtttgggaactgacacattggattaggtgatttctaggatgccttctagctagaataaagcccatcaccttccctggattccagccacagaaagaatagttaaattcttcatgtaattcagcaaggacgtactcagggtgctccagaagagaacactgggcataaagcagaagtcaagtcacattgatcatgaaggttaattttaggtatttcttgtcagtgtcctggtctcttctaaactctctggagctccattttttttttcttctgtaaaatgaggttaataggtgagtcccacctcataatgtcataaatctcataaatcacaccttatgggctttaaataagaaaatgtctaaaacgtttatcccaatgtctgaccacaatttgtacatgttagctgttgtcgttaatattataagatcaggaggcacagtgagattgtggttaaagtacttcagtgacaggtggaatgggttctaatttcagctgttcttgatagctctgtgacctcagtcaagttgctcaaattctctgaagctcagtttgctcatctctacaatgaggataattccagtgcccatgtcatgaggtggttgtaatgtataaacatgataatgtatgtaaaatgctcagcatagtacctggcccataaaaagcactcaactgtttattattactatagttactggttttagctctccaaagtttcagtctttggttagtctttagtgattggtagccaacatcttttatgcaattagagaggaaactaaatatggtggggacagaaaataataaagttataccagatacttattgtatgccattgttatattttaaagaatcttacaaccatagtgctagaagggacttcaagggtcatttagtctcactttcccatcctgtgcttcagtacttttttttttttttttaattgagacggagtcttgctctgtcacccagactagagtgcagtggcgcaatctcggctcactgcaagttccgcctcctgggttcacgccattctcctgcctcagcctcccaagtagctgggactacaggcgcccaccatcaagcccagctaatttttttcttttttgtatttttagtagagacagagtttcaccgtgttagccaggatggtctccatctcctgaccttgtgatcctcccgcctcggcctcccaaagtgctgggattacacgcgtgagccaccgcgcccggccagtactttttactacatcaccactgaatgggcatttgctgtagacggagttcactccctgtcgaggtggtgcatttccttatacttgtctctaccacagacagaagtcttctttcctttagtttttctttcctttttttttttttttgagacggagtctcactctctcacccaggctggagtgcaatgacacgatcttggctcactgcaaactctgcctcccaggttcatgccattctcctgcctcagcctcccaagtagctgggattacagacgcgcgccaccacgccccgctaatttttgtatttttattagagacggggtttcaccatggtggccatgctggtctcgaactcctaacctcaggtgatccctctgcctcggcctcccaaagcgctgggattacaggtgtaagccaccacgcccagccttccctttagtttttgtttgttagttccttccctctagggcaacaaaaatgggaatataggaataatacctcgaacctctgagcttctcaagggcctgactgagaaaaaatattgaaacaaccaaaaataggtcagtagctcctaaataggaaaataaaataaaaaattgaaatgaatgaatatttaatggtttgtctataatatgtaacattatgtaaattggctgctggcaaaccaactctaaatgtgggatgtgggagcatttaatataaaaatggggctgggcacagtggctcacatgtgtaatcccagcactatgggaggccaaggcaggcagatcactggagctcaggagttcaagaccagcctgggcaacatggcaaaaccccatctctactaagaatacaaaaaattaggctgagtgcagtggctcacacctgtaatcccagcactttgggaggccaaggcagatggatcacctgaggtcaggagtttaagccagcctggccaagatggtgaaacccctgtctctactaaaaatacaaaaattagctggtcatggtggtgcatgcctataattccagctactcaggaggctgaggcaggagaattgtctgagcctgggaggagtgaaccgagatctcaccactgtcctgaagcctggaaaacagagcgagattccatcccaaaaaaaaaaaaaaaaaaaaaaatagccgggcatggtggcatgtgcctgtagtcccagctacttggcagggctgaggcaggaagattgcttgaactcaggaggttgaggattcactgagccgagttggtgccactgtactccaaacctgggcaacagagcgagaccctttctcagaaaaaaaaaaaaaaaaaatggagtagagcctttaaaagtaagagatccaagggcctggaatttgaacacagaacaaatgcaacttgtcttctacctgaaggagtaaggactgggtcacaggtgagtcctcagctctagagtagatctgctttaaatcctagctttcccaattaatagctgtgtgatcttgggcaaagtggctctctcagactcagtatcttcatctgaaaaatggggaaaatagtagtacctacttattgtggttgaggattaaatgagattgtatgtatatggtgctaagcaccatgtctggcacctaataagtaccaaatagatggtaactactaaatttatacactgttaaggccatcacatctcacctccgctgctacctgccccaccaggcaaatcttggtttctccagaaagaacattgccactcccttcaactgtcccatccatcttggttggtggccaaatctgtgtgaaaaaaagctgtaatgacacaacagtgcttgattttactgatcattttcatagcaaaatgaagtttttaaaaagttagttttaccatttttatcaagaaaaataattataaggagtctgaaactggatttacatttaaaatcttatgtgcacttaagattcatgtttacttagcagttgtactgatgagggtcttttttttatttttttgatatggagtcttgctctgtcaccaggctggagtgcaatggtgtgatctcggctcactgcaacctccacctcctgggttcaagcgattccactgcctcagcctcctgagtagctgggactacaggcgtacaccaccacgcccggctaattttttgtattttagtagagatggggtttcaccatattggccaggatggtctcgatctcctgacctcgtgatctgcctgccctcggcctcccaaagtgctgggattacaggtgtgagccactgcaccaggcctgtggtttttatattttaaatttttttctggtaattagggcaaaagtaattggaacataatctttttttcttcaataattttaggctgaggctatgcctttataatatttaaactatagtttgctaaaccagcatttagtcaaaccctttatacacattaaggcttccagaaggtgacagtgacatttaagaagtttcaacatctctgactcagccataaaaatgagtaagcatgcttagttttccaaaagctggcaatgtacttatttttagaattcagtgttcatgaggtcagggcctgtgcccctcttgtttcctgctctgtcccctgtgcctagagtaagggcatagtgcatattcatggaatggatcagcagatgaagaaataaacaaatgaacaggaaggaaagatggtgagtgggtggctgggcaacgtgaccctcttcataatgacagcactgaggtaagggctggtgaaggcagtataattaagaatgtgcatttaaggctatgcagacctaaattggaaggccattttgtcatttgctaagtcatgtgaccttgggttagtcacttcacttccctaattttcagtttcttcatctgtgcaatgggactaacagtctctactttagaagcgtagttctaaagtaagtgcttctaagtatctctaaaatgagcttcttcactgaaagagtttatttagcacaaggcctggctcctattacattgtgaccacagtgattattttcatttgttggcaaccctgtctcccttctcatgagtaaatgcaaaacttatttaagaaaatggaggctgggcacggcacggtggctcacgcctgtaatcccagcactttgggaggccgaggcgggtggatcacttgaggtcaggagttcgagactagcctggccaacatggcgaaaccccgtctctactaaaaatacaaaaattagctgtgtgtggtggtgtgcccctgtagtcccagctaccagggaggctgaggcaggagaatcgcttgaactcaggagatggaggttgcagtgagcagagatcatgctactgcactccagcctgggtgatagagtgagattctgtctcgcaaaaagaaaaaaaagaaaaagaacatggaggttgcagtgagttgagattgtgccactgcactccagcctgggcaacagagtgagactgtctcaaaaaaacaaaaaagaaggaaatgaatttttgttgtcttttaaaatctaattagaatataatgcctgaggccaagcctggtcgttcacacctgtaatcccagcattttgggaggctgaggtgggcagatcacctgaggtcaggagttcacaaccagcctggtcaacatggcctcgtctctactaaaaatacaaaaattagctgggcgtggtggtgcatgcatgtagtcccagctacttgggaggatgagggatgagaattgcttgaaccagggaggccgaggttgcggtgagctgagattatgccactgcactccagcctgggtgacagagtgaaattctgtgtctaaataaataaataaataaataatgcctgagtcttatcaatattgctagatgtttgtagaactcattagggtcctgtgaaaaaaacaaagcacatatttaatgtaggacttcctggcagaagtctctatttcataaaacagtacggttcattatctagatgaaaaaatgtggccatcctaaagttgattcctgggtgttcagtaaattttgtctgtctctccattaattattgtgacaagtacggagtagaattcatttcaaataggattataggcatcattaagattttttaaaaacttagataaaagaaaacgttcttaactatactattggctatttttcctctctctctctctctctctctttttatagcagagacacagacactggggagcctccctgatataaatctaacagtaagtaactttttttgtgagctatgttgtactcaacttggaggggaaatcatggctttagacttccttcatgcccaccttgtccttggggtaaaaagccttaagttcccttagcttcagattcctgggctgtaacatgttgatgatgacagttctaacctcataggcctgctgtaatgaaggactgacatgatgtgtgtaaagtgcctggcatatagtaagagcttgaaaaccacaaagaagaaacattagcattttgtccctcttattcttcaatttcacaactgtctaccctgtggaaggttcaaagattcttcatctccctagaataccattcttcttgcttccattgcaatcctaatgggaaagagtatttaaatttatctctttaaatatggcaagacaccagtcaggaatattcaaagtgaagaggttttttttttttttttttagacagaatttcgctcttgttgcccaggctggagtgcaagggtgtgatcctggctcacctcaacctccgcctcccgggttcaagcgattctcctgcctcagccttccgagtagctgggattacaggcatgagccaccacacccggctaatttttttatttttagtagagacggagtttcaccacattggccaggctgatcttgaactcctgacctcaggtgatccacctgcctcggcctcccaaagtgctgagattgcaggcgtgagccatggcacctggcctaattttgtatttttagtagagacagggtttctccatgttggtcaggctggtcttgaactcctgacctcaggtgatccgcccaacttggcctcccaaagtgctgggattacaggcatgagccatggtgcccagccagttttatgtatttaactataaataaagccataccttaataaacctcaattttactgtccctgctaccttctcagaataaaagaaaaactggctgggcgcagtgactcacgtctgtaatcccagcactttgggaggctaagtagggtggatcacctgtcaggggtttgagaccagcctggtcaacatggtgaaaccccatctctactaaaaatacaaaaaattagccaggtatggtggtgtgtgcctgtgtagtctcagctactcaggaggctgagacaggagaatcgcttgaatccaggaggtggaggttgcagtgagctgaggtcgcgccactgcacaccagcctgggcaacaagagcgaaattccctcataaaagaaaaaccactagaaggcaagcttgcagcaatgacttaaaaattctttgaggaggccgggcacagtgtctcatgcctgtaatcccagcactttaggaggccaaggtggaaggaatgcttgagaccaggagtttgagactagcctgggccacatagtgacaccctgtctctacaaaaaatattttttaaaatttggccaggcatggtgctgcaaacctgtagttccagctacttgggaggctgaggcaggaggatcacttgagcccagtaggttgaggctgcattgagctgtgatcatgcccctgcactccatcctaggtgacagggcaaggccttgtctcaaaaaaaaaaaaaaaaaaaaaattacttgagggcatatccaggagcccacatttatttgactcagtttcttgcttgtgtattcacaactgatgctcagcatcttgtatttgctccttctctctttgcaatcaaacagtgctttttaaggacctgtatgtgcccacaatgcttaatgcacaatatcttgcatataggactcctgttattgcaggagtaatgtgtattgaatcaatttgtaaatgtatattgaataaatggaagatttaacttccttaaattcattgttttaaaaatgcttataattatatgtgtgtgtgtgtgtgtgtgtgtgtgtgtgtgtgtgttttgtataaatggggtctcaatattattaggctggtccctaactcctggtctcaagtgatcctcccatctcagcttcccaaagtgctgggattataggtgtgagcctggcctataattattatttatttattaatttattatttatttttgagatggagttttgctcttgtcgcccaggctggagtacaatggcacaaccttggctcactgcaacctctgcctcctgggttcaagcgattgtcctgcctcagcctcccaaatagctgggactataggtgcccgccaccacacctggctaatttttgtatttttagtagagatggggtttcaccatgttggccaggctggtcttgaactcctgacctcgtgatctgcctgccttggcctcccaaagtgctgggactacaggcgtgagccaccgcacctggcctaatcattgtttttaaagagagaaaaagagagaacccatagactgaaattttagaaacaggatttattttgtgtggctctcagttatcagaaaatcaagttaactagagcaatagcaataatagctacgattgattgaacatttattatgtaccaggccctgtactaagcattttatattttctctcacttagttttcctgataactctacaggattactcagcccttagtggcagagctgggaattttttttttttttttttttttgagatggagtctcgctctgttgcccaggttggcgtgcagtggcatgatctcaactcgagtactccagtctttgcctcctgggtttaagtgattctcctgcctcagcctccccactagctaggattacaggcatgtgttaccacgcctggctaagttttgtatttttagtagagacggggtttcaccatgttgtccaggctggtctcaaactcctgatgtcaggtgatccgcctgcctaggcctcccaaagtgctgggattacaggcattagccaccgcgcccggcctcagagctgggatttgaatccacgaccgtctggctgtaaagtcttggctattgaatcactgtctttttgtgaaaaatagacgtttcatagcaagacactaaatgtaaactactctaaaactctctggggaatcttaggggatcttgaaccaacaagactccactctaaagccaatttttgcaggggcagggaaagggtcttaagagctccttctctctttttttttttttttttttaagagctccttctctacccacaacacagaagggcacattttgtttccttgctaactccaaaggttaagggagaccctttcttgggttggcttgagtgttggctacaaggtgacagatcaaaggtgatttgaaggactcagccatcctgtgatgttgtaaaagggatggtccaattcacaaatcttgaagctgattacacaagcgatttctagctgttttctgcttaaatcctgtttgtgtttccatagcaaatatccacattggccaatgagttacgggactctagattggccaatgacttacgggactctggagcaaccatcagaataggcatctacatcggagcagggatctgtgctgggctggctctggctcttatcttcggcgctttaattttcaaatgtaagtgtttttgtttctctctccctctgataacaaattcttcagtgagaaccagtaaaaacctagatagaaatgcaaattcatgtttatacccaaagatgattagactaaatacccttttgtccatccaaatcctaacaataatggctaatttttatttaatatttattgtgtgtcaggcagccactgcgttaaatgttttacatacattttatcatcattactgtcttatgagataaggaatagtaccatccccattttacagtgaagggaagtcaagcttagagtaagctgccttcccgaggtttcaacatcagtacctggcagaaccggaaatcaaatccaggcaggctgattccagtaaactaatttctttaattcatgaatatgtctaggtttgttacgaaacacaacaacaaaaaacacttccttctgtgcacttttctgttattggtttctcaggcatacctctctggcagtagactgcaagtccctttaacactgggtcgtgactggggttagagaatgttttcctccatcatggtacttagcacatagctttttctaagttacttatcccttttttttttttaaactagcaaacaaacaaaaacaaaaaaacaggcccggcgcagtggctcacgcctgtaatcccagcactttgggaggctgaggggggtggatcatttgaggtcaggagttcgagaccagcttggccaacatggtgagaccccatctctactaaaaatacaaaattagccgggtgtggtggcgcatgcctgtaatcccagctactcaggaggctgaggcagaggaatcactcaaacctggtaggtggaggttacagtgagctcagatcatgccactgcactccagcctgggtgacagagtgagactgtcttaaaaaaaaaagtaataaaatataaataaataagcaaataaataaataaataaatacaatggccaggtgcagtggctcatgcctgtattttcaacactttcggaagctgaggtgagaggatcagttgagcccaggagtttgagaccaagatcagcctgggcagcaaagcaggatcctgcctctacaacgtaataataataataatgagctgggcatggtggcacacacctgtagtcccatctactcaggaggctgaggtgggaggatcacttgagctcaggaggctgcagtgagcggtgattgcatcattgcactccaccctgggcaacagaataagacagtctccaaaaaaaaaaaaaaaaagaagtgtgttgaagttgatgaattttgatggagaattacagtaggaagccatcatcacaatcaagatctaaatacttccagtacccccaaaacctcgcacacttttgtttttttggagatggacgctggagtgcagtggcacgatctcagctcactgtaacctctgcctcccgggttcaagtgtttctcctgactcagcttccagagtagctgggactacaggcatgtaccaccacgcctggctaatttttatatttttagtagagacagggtttcaccatgttgggcaagatgttcttgatctcctgaccttgtgatccgtccacctcgacctcccaatgttttgggattacaggcgtgagccactgtgcctggccactcacacacttttataattgtgtctattttattttcttttagggcagaaaccaagtccttatgccccagagcgtaatatgacatagcagcctttcttactaatacacaaacagggactgtgtgtgtagagtgagcaatcacctcagattagctcaagactaaagggtgttcctaagatatatgggacttctagggctaaaactggaacagtccaagaaaaacatgagttggtccccccactaactgtggactgaagctgccaagtgctttacgtgcattaaattctcgtaactaccctatgagataggtactaatatgtttctaatggagaatcagcatgctaaaaggttaggtcacttgcctgcatttacacagttaattgatagatactggatttgaaaccaggactgtcctgctcctcatctttatggtttctgcgattatggttaaatggattattcagaaaaagaaactaccccacccacacttacctgggtttattcagagatagccatggaatttttgaattgatcacatgaatatatatatatgtatatatatgtatacacatatatatatatatttattattattgggacagagtcttactctgtcacctgggagctggactacagtggtgtgatcttggctcacttcaacctctgcctcctgggttcaagcaattcttgtgcctcagcctcccaagtagctgagattacaggcatatgccaccacacctggctagttttttttttttttttttttttttttttttttttttttttggtagagacagggttttgccatgttgcccagaaaattggtctcaatctcctgagctcaggtgatcctcctacttcagcctcccaaagtgctgggattacaggcgtgatgaatatatacatatttaaaaatagaggctgagcacggtggcttacgcctgtaatcctagcactttgggaggccgaggtgagtggataacctgaggctgggagtttgagaccagcctgaccaacgtggagaaacctcatctctactaaaaatacaaaattagccgggcatgggggcacatgcctataattccagctactcgggaagctgaggtgggagaatcacttgagcccaggaggcggaggttgcagtgagctgagatcgtgccattgcactccagcctgggcaacaagagtgaaactccatctcaaaaaataaataaaataaaataaaataaaaatagaaatgagatcttgctatgttgctcaagctggtctcaaactcctgggcttaaataatcctcccacctcagcctcccaaaatgttgggattataggcatgagtcattgcacttagcctcacttgagtatttccaaaacagactagggattgttaaggagcactgataacactcactacttttttgacttctgccatggctcaaagtaaaactttgagctttactattatatatattagagaccactttatatatatatgtgtgtgtgtatatatatatatatatatatatataatatgaccataatttatacatattagcacttattctagtttgtataaagagtgatgggatctagaggaaatctgatgctttgtacacacacatatatgccactccaaaaaaacatggctactgagaagtacagataagaaaggcctgtttcatttccaatcttttccctggaagttgctggagaaaatattaaaatcaaggtgacagctttttaagtcatgacctcacaaaaacaaactgacactcaataatgccccacaatcttccatctttctagtgtactctctgagatgaccatccatacctcactgctgctcaaaacttctttgcccctctcctaactgatgaaatatgaaagggctttttaatctccattgcctaggccaagacttcatccttctttttatccctttcttctgagtcatctctctccttttctgtttatgctattttcattagcagaatatatgttctagtccatgcattcctctccagctagtcccatttctcaactcgttctcacagccatatttttcaaaggagttgtctctacttttcacccatctacactccttgacccaccatcatctagtttctgctccaaagcacaacggaaatggtcaagctcaccactgaccttcagttttctaaattaaatattaacagatacctttatctttttttttttttttgagacagagtttcactcttgttgcttaggctggagtgcaatggtgcaatcttggctcactgcaacctctgcctccccgggttcaagtgattctcccccctcagcctcctgagtagctgggatttcatgcatgtgccgccatgcctggctaattttgtatttttagtagagacgggggtttcaccatgttggccaggctggtctcaaactcttgacctcagatgatctgcccgccttggcctcccaaagtgctgggattataggcgtgagccaccgttcccagcctgatacctttatctttattatagtcaaccttttaaattgatcactcattctagaaagtcctccttctcttggcttctgaaatgacattcttctctttgatccattgcttttcagatcttcttctgcctgatccttagatgttgagattccttagggctgggtatggtggtgcacgcctatcgtcccagctactcaggagaatgaagcaggaagatcacttgagcccaggagtttgaggctatgaactatgatcttgctgctgcattctagcctgggtgacacagcacagggagaccctgtctcagagaaaaacaacaacagaagattcctgcagtcttcctccaacatacatctaaagtgttttcattattctgcatctcttttggccattccttactccaagacaccattatttctacttaatttatcttctcatttctttattcctccttcgaatccaatattaaaacccaaatcaatgtctgagcacaatggctcatgcctataatcctaacacttcgggaggccaagacaggagagtcacttgaggccaggagtttgagactagcctgggcaacatattgagattcacccaggctggagtgcagtggtgtgatcatggctcactgcaacctctacctcccgagttcaagcaactctcctgcctcagcctccaagtagctaggaccacgggcatgtgccagcatacctggctagtttttgtatttttagtagagatgaggttttaccatgttgcccaggctggtctcgaactcccaagctcaagaaatccacctgccttgcctcccaaagtgctaggattacaggcgtgagccactgcatccagcccaaaaacattaaaaaaaattatccagctgtggtagtgtgcacctatagttctagctactcagaagctgagatgggtagatcccttgagcccaggagttcaaagttgcaatcagatatgatcttactactgcattccagcctggccaacagagcaagaccctgtctcaaaaaaaaaaaaaaaaaaaaaaaaagaaaagaaaagaaaggaaaattttaagtttcaaatttttgaaactgcatccccagaagacaccttgaaagaccttgatccagaggtacccagtaagccacacctggattcctgacccacaacacagaaatgatgagataacgtagttgttttcagcgggtaaaagaaacccaaaacaaataaggatcacatctctgcccttcaaggaaatctaaggtttcctacaggtcttagaataaaatccagactcctgattatggctggaaaggtcttattggacctagccccagcctacctttcagaccttatcttgggccgctcccttaatccagccacgttttcagtttcaggtactcagcaagatctttacaaggggctccctcatcctggaatgctccttctggctctttacttggctgactcatttcagaaacgagcttaaagttcatctctgagggtccttccctgaccaccctctctattatctgtttcactatgatgttggtttcctttataatacatcataatttgaaattattttgttttcctatttactatttcctttttgttgttgtttttttttttttgagacggagtttcgctctcgttgcccaggctggagtgcaatggcgcaatcttggctcaccgcaacctccgtctcccgggttcatgtgattctcctgcctcagcctcccgagtagctgggattacaggcatgggccaccacatccagctaattttgtatttttagtagagacggggtttctccatgttggtcaggctggtcttgggtctggggtactgttttcttttatttctttggtctcccacaggagagcgtgagcttcgtgaaattagggctcacaattcacttttatgttcctgtgttccttgtgccaggcaaactgccagacacatagtagaggtctcataatatttgttaaataagaaaaatgcatctttattattattttctaacacagtccatataactatctaaggcacacgttgagagtttcatttaactcaatctgagatgagaccagatttccctaaactatgcgtgcttccagtgaacctatttgatttcttgtctcatttattttatgaatgcatttttattggcttttgtttcccccttactagggtattctcatagcaaagagaagatacagaatttaaggtaagaataatggaatggggttgggaaaaggtgagaatcttggaagtccctactctaacatgtctgatcttttctctgagaaatcctgtcctggggctttgagatctgagtttatacttccttctttctctaggtctagttatgaagtgaaaatgtaacatgctactgtgtgtacttcctgacacctctagcttctttagttctaaagttaaggtatagaatttatcaccctaataatacaacatgcttattcatctcgatattaatggtttttccactgaactgaaacatcaacggattgagctcctattatatccagggtctgggctgcacttaggcgttacagaagtgagcaagaaacccagtatctgttctcagcatttctgtaaacagcccaggcattttcaactttagtttcctattagaatcacccaggcactgaagtgtactaatgcagggccaaccccagagcaactgaatcagaatctctggggatgaagcctaagcatagaaattgttctaaaagctctccaggtgattctagtgaacagtcagggctgagaactgctagtctgttatagactcctgtcttgaccttcgttcctgattatacacacccacacatccatttcaaataggtatggcttacataacttactagattttagacttttagtgatgtttggcacatgaatggaatacaatatatttggattacaaaaatgattctgggctaggtgctgtggcttacgcctataagcccagcactttgggaggctgaggtgggaggatcacatgagcccaggagttcaagaccagcctgggcaacatgatgagacctcttctctacaaaaaaatggaaacattaggtggaaggattgctggagcacaggagatgaaggctgcaatgagccatgattgtgccactgcattcccacctgggtgacagagaaagacccagcatcaaacaaaacaaaacaaaaacctctgtccattgaatgctatctaaagcattaataattttttttttttttttttgagacggagtctggctctgtcacccaggctggagtgcagtggcacaacctcggctcactataacctctgcttctcgggttcaagcgattctctgcattagcctcccaagtagctgggattacaggtgcatgccaccacgctggctaattttttgtatgtttagtagagacggggtttcactatgttggccaggctggtctcgaactcctgacctcgtgatctgccaggctcagcctcccaaagtgctgggattacaggcgtgagccacggcgcccggcccattttttgaaaatctaaatgtgtacataattgcagacatcttgatacttcacctaaacttcagcctgcacctccgagaaataaaaatattctcttagctatcacaatgctgttgttatacctaagaaaattaaaattaatggacattttctcaattgtttccccatagtattttattgctatttcttggcttttccacttgttcagcatctgatcaaagatcatgcactatatttagttgctgtctctttgggaaagcctattctcttaactctattccgtgttacctgggaaatgcttaactcaaccgcaacactttatttccacagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttttttgtttgtttgtttgttttttgagatagaatctcgctctgtctgagttcattgtagattctggatattagccctttgtcagatgagtaggttgcgaaaattttctcccattttataggttgcctgttcactctgatggtagtttcttttgctgtgcagaagctctttagtttaattagatcccatttgtcaattttggcttttgtggccattgcttttggtgttttagacatgaagtccttgcccatgcctatgtcctgaatggtaatgtctaggttttcttctagggtttttagggttttaggtctaacgtttaagtctttaatccatcttgaattaatttttgtataaggtgtaaggaagggatccagtttcagctttctacatatggctagccagttttcccagcaccatttattaaatagggaatcctttccccattgcttgtttttctcaggtttgtcaaagatcagatagttgtagatatgcggcattatttctgagggctctgttctgttccattgatctatatctctgttttggtaccagtaccatgctgttttgattactgtagacttgtagtatagtttgaagtcaggtagcgtgatgcctccagctttgttcttttggcttaggattgacttggtgatgcgggctcttttttggttccatatgaactttaaagtagttttttccaattctgtgaagaaagtcattggtagcttgatggggatggcattgaatctataaattaccttgggcagtatggccattttcaccatattgattcttcctacccatgagcatggaatgttcttccatttgtttgtatcctcttttatttcattgagcagtggtttgtagttctccttgaagaggtcctttacgtcccttgtaagttggattcctaggtattttattctctttgaagcaattgtgaatgggagttcactcatgatttggctctgtttgaactcaaacaaatttacaagaaaaaaacaaacaaccc

SEQ ID NO: 46 - protein, 236 aa, synthetic construct; identical to SEQ ID NO: 32

SEQ ID NO: 47 - protein, 286 aa, synthetic construct; identical to SEQ ID NO: 33

SEQ ID NO: 48 - protein, 238 aa, synthetic construct; identical to SEQ ID NO: 31

SEQ ID NO: 49 - protein, 236 aa, synthetic construct; identical to SEQ ID NO: 32

SEQ ID NO: 50 - protein, 286 aa, synthetic construct; identical to SEQ ID NO: 33

SEQ ID NO: 51 - protein, 236 aa, synthetic construct; identical to SEQ ID NO: 32

SEQ ID NO: 52 - protein, 286 aa, synthetic construct; identical to SEQ ID NO: 33

(19) United States
(12) Patent Application Publication
(10) Pub. No.: US 2025/0354167 A1
(43) Pub. Date: Nov. 20, 2025

(54) VECTOR

(71) Applicant: ZYGOSITY LIMITED, Cambridge (GB)

(72) Inventors: Iain Alasdair Russell, Cambridge (GB); Stephanie Mack, Cambridge (GB)

(21) Appl. No.: 18/716,753

(22) PCT Filed: Dec. 8, 2022
(86) PCT No.: PCT/EP2022/085065

§ 371 Date: Oct. 16, 2024

Related U.S. Application Data
(60) Provisional application No. 63/287,720, filed on Dec. 9, 2021.

(30) Foreign Application Priority Data
Aug. 26, 2022 (GB) 2212476.2

(51) Int. Cl. C12N 15/86; C07K 14/705; C07K 14/725; C12N 9/22; C12N 15/11; C12N 15/90

(52) U.S. Cl. CPC C12N 15/86; C07K 14/7051; C07K 14/70521; C12N 9/226; C12N 15/11; C12N 15/907; C12N 2310/20; C12N 2740/15043

(57) ABSTRACT

Provided herein is an expression vector comprising a coding nucleic acid sequence encoding a polypeptide of interest, wherein the polypeptide of interest is not constitutively expressed from the vector; and a guide-binding sequence located upstream of the coding sequence, wherein the guide-binding sequence comprises a sequence complementary to a nucleic acid guide; wherein binding of a nucleic acid guide to the guide-binding sequence directs a mutation in a nucleic acid sequence of the vector resulting in expression of the polypeptide of interest. Also provided are a combination comprising the expression vector and a nucleic acid guide, a cell comprising the expression vector and/or nucleic acid guide and associated medical methods and uses.

### STATEMENT OF RELATED APPLICATIONS

This application is a national phase application under 35 U.S.C. § 371 of PCT International App. No. PCT/EP2022/085065, filed Dec. 8, 2022, which claims the benefit of GB Patent App. No. 2212476.2, filed Aug. 26, 2022, and claims the benefit of U.S. Provisional Patent App. No. 63/287,720, filed on Dec. 9, 2021, each of which is hereby incorporated by reference for all purposes as if fully set forth herein.

### SEQUENCE LISTING

The text of the computer readable sequence listing filed herewith, titled “P6145GBWO-seql-000001”, created Dec. 2, 2022, having a file size of 261,226 bytes, is hereby incorporated by reference in its entirety.

### FIELD OF THE INVENTION

The present invention relates to the fields of biotechnology and products for use in medical treatment. In particular, the present invention relates to methods for expressing an exogenous gene in a cell, as well as simultaneously ablating or disrupting expression of an endogenous gene in the cell. The present invention includes expression vectors, combinations of such expression vectors with nucleic acid guides, isolated cells comprising the expression vectors and/or nucleic acid guides, as well as methods and uses thereof. In one aspect, the present invention relates to uses of such vectors, combinations and cells to treat diseases such as cancer.

### BACKGROUND OF THE INVENTION

Current methods for expressing an exogenous polypeptide in a cell are cumbersome and not easily controllable. Expression vectors such as adeno-associated viruses (AAV) are useful for introducing an exogenous gene into a cell. Whilst expression of a polypeptide from the exogenous gene may be under the control of various constitutive or inducible promoters and other regulatory elements, a level of expression of the polypeptide in the cell may vary depending on the cell type and other external factors. Thus it is not always straightforward to provide an expression vector in which polypeptide expression can be easily and rapidly switched on and/or off.

Rapid and efficient control of gene expression from a vector may be particularly important in the context of methods that also involve a step of inactivation or disruption (‘knock-out’) of one or more endogenous genes. An example of such a method is CAR (chimeric antigen receptor) T cell therapy for diseases such as cancer. In such methods, donor T cells are obtained from a subject and are modified ex vivo to express a CAR (e.g. directed against a tumour antigen) before being returned to a recipient. The CAR may be introduced into the T cells using an expression vector such as a recombinant AAV, and is integrated into the genome of the cell. The method may further involve inactivating one or more endogenous genes in the T cells, e.g. those encoding endogenous T cell receptor sequences and/or immune checkpoint molecules (such as PD-1).

In such methods, CAR integration and endogenous gene silencing are performed in separate, linear steps. For instance, an AAV vector encoding a CAR gene may first be introduced into the T cells, followed by purification of cells which express the CAR polypeptide at sufficient levels. The gene silencing step is then performed on the purified CAR-expressing cells, e.g. using a gene editing tool such as CRISPR-Cas9. This method is slow and inefficient, with ever diminishing returns, because only a small fraction of the original T cells efficiently express the CAR and are purified in the first step, and then only a small fraction of the purified cells show effective silencing of the endogenous genes.

Particularly, coediting of polypeptides in a cell (e.g., expression of one polypeptide and switching off expression of another polypeptide through deletion or frameshift mutations) tend to be performed in sequential steps that have diminishing returns. Accordingly there is a need for improved expression vectors and associated methods for controllable expression of an exogenous polypeptide in a cell. Moreover, there is a particular need for improved vectors and methods for expressing an exogenous polypeptide in a cell, and simultaneously or concurrently inactivating or disrupting expression of one or more endogenous genes in the cell. There is also a need for improved products, compositions and methods of treating diseases such as cancer, e.g. in the context of CAR T cell therapy, in which both expression of an exogenous gene (e.g. a CAR) and inactivation or disruption of endogenous genes (e.g. encoding T cell receptor polypeptides and/or immune checkpoint inhibitors) is performed more efficiently.

### SUMMARY OF THE INVENTION

Embodiments of the present invention address the problems discussed above. In particular, according to one aspect of the present invention an expression vector is provided which encodes a polypeptide of interest. In embodiments, the polypeptide of interest may not be constitutively expressed, for example, by placing the nucleic acid sequence encoding the polypeptide out of frame with a start codon. The expression vector may further comprise a sequence complementary to a nucleic acid guide, e.g. an RNA guide capable of directing Cas9-mediated gene editing. In embodiments, the nucleic acid guide is selected such that it directs or initiates a predictable and consistent gene editing event (i.e. mutation) at a sequence in the expression vector. The mutation initiated by the nucleic acid guide results in a change in the expression of the polypeptide of interest from the vector, e.g. by providing a frameshift mutation that shifts the coding sequence of the polypeptide of interest. For example, in embodiments, the mutation initiated by the nucleic acid guide is a frameshift mutation which shifts the coding sequence of the polypeptide of interest into frame with a start codon, resulting in expression of the polypeptide of interest.

Thus expression of the polypeptide by the vector can be rapidly and precisely controlled, including after the expression vector has been introduced into a cell and optionally integrated into a host genome, using the nucleic acid guide. In particular, since the guide-binding sequence present in the vector may be specifically selected such that a corresponding nucleic acid guide produces a desired mutation, changes to the expression (e.g. switching on expression) of the polypeptide can be initiated by introducing the nucleic acid guide into the cell, e.g. in combination with a gene editing tool (such as Cas9).

Moreover, in embodiments the present invention can further be used to inactivate or disrupt expression of one or more endogenous genes. For instance, the guide-binding sequence present in the expression vector may be selected such that it is also present in one or more endogenous genes. Thus after the expression vector is integrated into a cell, a single nucleic acid guide may be used to both activate (i.e. switch on) expression of the polypeptide of interest from the vector and disrupt (i.e. switch off) expression of one or more endogenous genes present in the cell. In some embodiments, the nucleic acid guide may result in the same mutation in the expression vector as in the endogenous gene (e.g. a one or two nucleotide insertion). However whilst the mutation in the expression vector activates expression of the polypeptide of interest (e.g. by shifting the coding sequence into frame with a start codon), the mutation in the endogenous gene disrupts expression.

Thus in some embodiments, this enables substantially simultaneous or concurrent activation of expression of the exogenous gene and inactivation of expression of an endogenous gene. In other words, the exogenous and endogenous genes can be ‘co-edited’ under the control of a single guide to modify the expression profile of the cell. Such a method is particularly useful in methods involving ex vivo modification of (e.g. mammalian) cells for use in human or animal therapy, especially CAR T cell therapy. For instance, co-editing of both a CAR gene integrated into the host cell genome and an endogenous gene using the single RNA guide and Cas9 in a single step avoids the need for two separate and sequential purification steps, i.e. following CAR integration and then gene silencing. Thus according to embodiments of the present invention, T cells which both express the CAR and lack endogenous T cell receptor and/or immune checkpoint molecule expression can be purified in parallel in a single step.

Accordingly, one aspect of the present invention provides an expression vector comprising: a coding nucleic acid sequence encoding a polypeptide of interest, wherein the polypeptide of interest is not constitutively expressed from the vector; and a guide-binding sequence located upstream of the coding sequence, wherein the guide-binding sequence comprises a sequence complementary to a nucleic acid guide; wherein binding of a nucleic acid guide to the guide-binding sequence directs a mutation in a nucleic acid sequence of the vector resulting in expression of the polypeptide of interest.

In some embodiments, the guide-binding sequence is located on the opposing strand to the coding sequence and/or PAM.

In some embodiments, the coding nucleic acid sequence is out of frame with a start codon.

In some embodiments, the nucleic acid guide initiates a frameshift mutation at the guide-binding sequence.

In some embodiments, the frameshift mutation at the guide-binding sequence shifts the coding sequence into frame with the start codon, such that the polypeptide of interest is expressed.

In some embodiments, the frameshift mutation is an insertion or deletion of a number of nucleotide residues not divisible by three. In embodiments, the frameshift mutation is an insertion or deletion of one or two nucleotide residues, preferably an insertion of one nucleotide residue.

In some embodiments, the coding nucleic acid sequence is out of frame with the start codon by a defined number of nucleotide residues, and the frameshift mutation results in the insertion or deletion of the defined number of nucleotide residues, such that the frameshift mutation at the guide-binding sequence shifts the coding sequence into frame with the start codon.

In some embodiments, the guide-binding sequence is present in one or more endogenous genes, preferably one or more mammalian genes, more preferably one or more human genes.

In some embodiments, binding of a nucleic acid guide to the guide-binding sequence in an endogenous gene disrupts expression of the endogenous gene.

In some embodiments, binding of a nucleic acid guide to the guide-binding sequence in an endogenous gene initiates a frameshift mutation in the endogenous gene.

In some embodiments, binding of a nucleic acid guide to the guide-binding sequence in the expression vector initiates the same mutation as binding of the nucleic acid guide to the guide-binding sequence in an endogenous gene.

In some embodiments, the endogenous gene(s) encode an immune checkpoint molecule.

In some embodiments, the endogenous gene(s) are selected from: BAF chromatin remodelling complex subunit BCL11A (BCL11A, ENSG00000119866), hemoglobin subunit alpha 2 (HBA2, ENSG00000188536), T cell receptor alpha constant (TRAC; ENSG0000027734), programmed cell death ligand 1 (PD-1; ENSG00000188389), cluster of differentiation 38 (CD38; ENSG00000004468), cluster of differentiation 39 (CD39; also referred to as ectonucleoside triphosphate diphosphohydrolase 1, ENTPD1; ENSG00000138185), T cell immunoglobulin and mucin domain-containing protein 3 (TIM3; also known as hepatitis A virus cellular receptor 2, HAVCR2; ENSG00000135077), T cell immunoreceptor with Ig and ITIM Domains (TIGIT; ENSG00000181847), lymphocyte activating 3 (LAG3; ENSG00000089692), T cell receptor beta constant 1 (TRBC1; ENSG00000211751), T cell receptor beta constant 2 (TRBC2; ENSG00000211772), cytokine inducible SH2 containing protein (CISH; ENSG00000114737), cluster of differentiation 70 (CD70; ENSG00000125726), beta-2-microglobulin (B2M; ENSG00000166710), major histocompatibility complex, class I, A (HLA-A; ENSG00000206503), major histocompatibility complex, class I, B (HLA-B; ENSG00000234745), major histocompatibility complex, class I, C (HLA-C; ENSG00000204525), major histocompatibility complex, class I, E (HLA-E; ENSG00000204592), major histocompatibility complex, class I, G (HLA-G; ENSG00000204632), killer cell lectin like receptor C1 (KLRC1, also referred to as NKG2A; ENSG00000134545), killer cell lectin like receptor K1 (KLRK1, also referred to as NKG2D; ENSG00000213809), Cbl proto-oncogene B (CBLB; ENSG00000114423), transforming growth factor beta receptor 1 (TGFBR1; ENSG00000106799) and transforming growth factor beta receptor 2 (TGFBR2; ENSG00000163513); preferably wherein the endogenous gene comprises PD-1, TRBC1, TRBC2 and/or TRAC. In embodiments, the endogenous nucleic acid sequence is an intragenic or intronic nucleic acid.

In some embodiments, the guide-binding sequence does not comprise a premature or alternative STOP codon.

In some embodiments, the guide-binding sequence is 8 to 50 nucleic residues in length, preferably 15 to 35 nucleotide residues in length, more preferably 15 to 25 nucleotide residues in length.

In some embodiments, the coding sequence encodes a chimeric antigen receptor.

In some embodiments, the guide-binding sequence binds to a nucleic acid guide.

In some embodiments, the nucleic acid guide is located within a guide RNA.

In some embodiments, the guide RNA further binds to an enzyme, preferably an endonuclease.

In some embodiments, the nucleic acid guide directs a gene editing tool to produce the mutation in the expression vector and/or the endogenous gene.

In some embodiments, the enzyme or gene editing tool is selected from: a Zinc finger nuclease (ZFN), a Cas enzyme, or a transcription activator-like effector nucleases (TALENS), preferably wherein the enzyme or gene editing tool is a Cas enzyme, more preferably Cas9.

In some embodiments, the guide-binding sequence further comprises a protospacer adjacent motif (PAM). For instance, the guide binding sequence may be located adjacent to a PAM or complement thereof. In embodiments, the guide binding sequences are located downstream (3′) to a sequence complementary to a protospacer adjacent motif (PAM) or complement thereof.

In some embodiments, the PAM is selected from: NGG, NGA, NGAN, NGNG, NGAG, NGCG, NGN, NRN, NYN, NG, GAA, GAT, NNGRRT, NGRRN, NNNNGATT, NNNNRYAC, NNAGAAW, NAAAC. NNG, or NNGG, preferably wherein the PAM is NGG or NGA, wherein N is A, G, C or T, R is A or G, Y is a C or T, and W is A or T.

In some embodiments, the guide-binding sequence comprises: SEQ ID NOs: 13-15, 22, 27-30 and 42, preferably SEQ ID NOs: 13, 15 or 27.

In some embodiments, the nucleic acid guide is 8 to 50 nucleic residues in length, preferably 15 to 35 nucleotide residues in length, more preferably 15 to 25 nucleotide residues in length.

In some embodiments, the nucleic acid guide comprises or consists of: SEQ ID NOs: 2-3, 23-26, 36-37 or 41 preferably SEQ ID NOs: 2, SEQ ID NO: 23 or SEQ ID NO: 25.

In some embodiments, the vector is an adenovirus, retrovirus, adeno-associated virus or lentivirus.

In some embodiments, the vector is an integrated expression vector.

In some embodiments, the vector further comprises a promoter sequence, nuclear localisation signal, a Kozak sequence, a sequence encoding a reporter gene, and/or a sequence encoding a 2A peptide.

Aspects of the present invention also provide an expression vector comprising: (a) a coding nucleic acid sequence encoding a polypeptide of interest; and (b) a guide-binding sequence located upstream of the coding sequence, wherein the guide-binding sequence comprises a sequence complementary to a nucleic acid guide, and wherein the guide-binding sequence is present in one or more endogenous genes; wherein binding of a nucleic acid guide to the guide-binding sequence directs a mutation in a nucleic acid sequence of the vector resulting in a change of expression of the polypeptide of interest; and wherein binding of a nucleic acid guide to the guide-binding sequence in the one or more endogenous genes disrupts expression of the endogenous gene.

In some embodiments, the guide-binding sequence is located on the opposing strand to the coding sequence and/or PAM.

In some embodiments, the mutation is a frameshift mutation.

In some embodiments, the nucleic acid guide initiates a frameshift mutation in the vector and/or the or one or more endogenous genes.

In some embodiments, the nucleic acid guide initiates the same mutation in the guide-binding sequence in the vector and the guide binding sequence in the endogenous gene.

In some embodiments, the frameshift mutation is an insertion or deletion of a number of nucleic acid residues not divisible by three. In some embodiments, the frameshift mutation is an insertion or deletion of one or two nucleic acid residues, preferably an insertion of one nucleic acid residue.

In some embodiments, the coding nucleic acid sequence is out of frame with a start codon.

In some embodiments, the frameshift mutation at the guide-binding sequence shifts the coding sequence into frame with a start codon, such that the polypeptide of interest is expressed.

In some embodiments, the coding nucleic acid sequence is out of frame with the start codon by a defined number of nucleic acid residues, and the frameshift mutation results in the insertion or deletion of the defined number of nucleic acid residues, such that the frameshift mutation at the guide-binding sequence shifts the coding sequence into frame with the start codon.

In some embodiments, the one or more endogenous genes are mammalian genes, preferably human genes.

In some embodiments, the endogenous gene(s) encode an immune checkpoint molecule.

In some embodiments, the endogenous gene(s) are selected from: BCL11A, HBA2, TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2; preferably wherein the endogenous gene comprises PD-1, TRAC, TRBC1 and/or TRBC2. In embodiments, the endogenous nucleic acid sequence is an intragenic or intronic nucleic acid.

In some embodiments, the guide-binding sequence does not comprise a premature or alternative STOP codon.

In some embodiments, the guide-binding sequence is 8 to 50 nucleic residues in length, preferably 15 to 35 nucleic acid residues in length, more preferably 15 to 25 nucleic acid residues in length.

In some embodiments, the coding sequence encodes a chimeric antigen receptor.

In some embodiments, the guide-binding sequence binds to a nucleic acid guide.

In some embodiments, the nucleic acid guide is located within a guide RNA. In some embodiments, the guide RNA further binds to an enzyme, preferably an endonuclease.

In some embodiments, the nucleic acid guide directs a gene editing tool to produce the mutation.

In some embodiments, the enzyme or gene editing tool is selected from: a Zinc finger nuclease (ZFN), a Cas enzyme, or a transcription activator-like effector nucleases (TALENS), preferably wherein the enzyme or gene editing tool is a Cas enzyme, more preferably Cas9.

In some embodiments, the guide-binding sequence further comprises a protospacer adjacent motif (PAM). For example, the guide-binding sequence may be located adjacent to a protospacer adjacent motif (PAM) or complement thereof. In embodiments, the guide binding sequences are located downstream (3′) to a sequence complementary to a protospacer adjacent motif (PAM) or complement thereof.

In some embodiments, the PAM is selected from: NGG, NGA, NGAN, NGNG, NGAG, NGCG, NGN, NRN, NYN, NG, GAA, GAT, NNGRRT, NGRRN, NNNNGATT, NNNNRYAC, NNAGAAW, NAAAC. NNG, or NNGG, preferably wherein the PAM is NGG or NGA, wherein N is A, G, C or T, R is A or G, Y is a C or T, and W is A or T.

In some embodiments, the guide-binding sequence comprises: SEQ ID NOs: 13-15, 22, 27-30 and 42, preferably SEQ ID NOs: 13, 15 or 27.

In some embodiments, the nucleic acid guide is 8 to 50 nucleic residues in length, preferably 15 to 35 nucleic acid residues in length, more preferably 15 to 25 nucleic acid residues in length.

In some embodiments, the nucleic acid guide comprises or consists of: SEQ ID NOs: 2-3, 23-26, 36-37 or 41, preferably SEQ ID NOs: 2, SEQ ID NO: 23 or SEQ ID NO: 25.

In some embodiments, the vector is an adenovirus, retrovirus, adeno-associated virus or lentivirus.

In some embodiments, the vector is an integrated expression vector.

Aspects of the invention also provide a combination comprising: (a) an expression vector as defined herein; and (b) a nucleic acid guide that binds to the guide-binding sequence and directs a mutation in a nucleic acid sequence of the vector resulting in expression of the polypeptide of interest.

Aspects of the invention also provide an isolated cell comprising the expression vector disclosed herein, or the combination as disclosed herein.

In some embodiments, the isolates cell further comprises a gene editing tool.

In some embodiments, the gene editing tool is a Cas enzyme, preferably Cas9.

In some embodiments, the cell is ex vivo. In some embodiments, the cell is a blood cell, a stem cell, immune cell, or dermal cell. Preferably, the cell is a T lymphocyte.

In some embodiments, the expression vector is integrated into the cell genome.

Aspects of the invention also provide a method of altering expression of a polypeptide of interest in a cell, said method comprising: (i) providing a cell with an expression vector as defined herein; and (ii) providing the cell with a nucleic acid guide complementary to the guide-binding sequence of the vector; wherein binding of the nucleic acid guide to the guide-binding sequence directs a mutation in a nucleic acid sequence of the vector resulting in altered expression of the polypeptide of interest.

In some embodiments, the altered expression of a polypeptide of interest is switching on expression of the polypeptide of interest.

In some embodiments, the method further comprises (iii) providing the cell with a gene editing tool.

In some embodiments, the gene editing tool is an endonuclease. In some embodiments, the endonuclease is selected from: a Zinc finger nuclease (ZFN), a Cas enzyme, or a transcription activator-like effector nucleases (TALENS), preferably wherein the enzyme or gene editing tool is a Cas enzyme, more preferably Cas9.

In some embodiments, the cell is ex vivo. In some embodiments, the cell is a blood cell, a stem cell, immune cell, or dermal cell. Preferably, the cell is a T lymphocyte.

In some embodiments, the expression vector is integrated into the cell genome.

In some embodiments, the nucleic acid guide initiates a frameshift mutation at the guide-binding sequence. In some embodiments, wherein the frameshift mutation at the guide-binding sequence shifts the coding sequence of the expression vector into frame with the start codon, such that the polypeptide of interest is expressed. In some embodiments, the frameshift mutation is an insertion or deletion of a number of nucleotide residues not divisible by three. In some embodiments, the frameshift mutation is an insertion or deletion of one or two nucleotide residues, preferably an insertion of one nucleotide residue.

In some embodiments, the nucleic acid guide is 8 to 50 nucleic residues in length, preferably 15 to 35 nucleotide residues in length, more preferably 15 to 25 nucleotide residues in length.

In some embodiments, the nucleic acid guide comprises or consists of: SEQ ID NOs: 2-3, 23-26, 36-37 or 41 preferably SEQ ID NOs: 2, SEQ ID NO: 23 or SEQ ID NO: 25.

In some embodiments, the nucleic acid guide is located within a guide RNA. In some embodiments, the guide RNA comprises a guide RNA scaffold, preferably a guide RNA scaffold comprising SEQ ID NO: 4.

Aspects of the invention also provide a method of expressing a protein of interest in a cell, and concurrently disrupting expression of one or more endogenous genes in the cell, said method comprising (i) providing a cell with an expression vector as defined herein; (ii) providing the cell with a nucleic acid guide complementary to the guide-binding sequence of the vector and complementary to one or more endogenous genes; wherein binding of the nucleic acid guide to the guide-binding sequence directs a mutation in a nucleic acid sequence of the vector resulting in expression of the polypeptide of interest, and wherein binding of the nucleic acid guide to the one or more endogenous genes directs a mutation to the one or more endogenous genes resulting in disruption of the expression of the endogenous gene(s) in the cell.

In some embodiments, the method further comprises (iii) providing the cell with a gene editing tool.

In some embodiments, the gene editing tool is an endonuclease. In some embodiments, the endonuclease is selected from: a Zinc finger nuclease (ZFN), a Cas enzyme, or a transcription activator-like effector nucleases (TALENS), preferably wherein the enzyme or gene editing tool is a Cas enzyme, more preferably Cas9.

In some embodiments, the cell is ex vivo. In some embodiments, the cell is a blood cell, a stem cell, immune cell, or dermal cell. Preferably, the cell is a T lymphocyte.

In some embodiments, the expression vector is integrated into the cell genome.

In some embodiments, the nucleic acid guide initiates a frameshift mutation at the guide-binding sequence. In some embodiments, wherein the frameshift mutation at the guide-binding sequence shifts the coding sequence of the expression vector into frame with the start codon, such that the polypeptide of interest is expressed. In some embodiments, the frameshift mutation is an insertion or deletion of a number of nucleotide residues not divisible by three. In some embodiments, the frameshift mutation is an insertion or deletion of one or two nucleotide residues, preferably an insertion of one nucleotide residue.

In some embodiments, the nucleic acid guide is 8 to 50 nucleic residues in length, preferably 15 to 35 nucleotide residues in length, more preferably 15 to 25 nucleotide residues in length.

In some embodiments, the nucleic acid guide comprises or consists of: SEQ ID NOs: 2-3, 23-26, 36-37 or 41 preferably SEQ ID NOs: 2, SEQ ID NO: 23 or SEQ ID NO: 25.

In some embodiments, the nucleic acid guide is located within a guide RNA. In some embodiments, the guide RNA comprises a guide RNA scaffold, preferably a guide RNA scaffold comprising SEQ ID NO: 4.

In some embodiments, the one or more endogenous gene(s) encode an immune checkpoint molecule. In some embodiments, the one or more endogenous genes are selected from: TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2, preferably wherein the endogenous genes are PD-1, TRBC1, TRBC2, and/or TRAC. In some embodiments, the nucleic acid sequence is an intragenic or intronic nucleic acid.

In some embodiments, binding of a nucleic acid guide to the guide-binding sequence in the expression vector initiates the same mutation as binding of the nucleic acid guide to the guide-binding sequence in an endogenous gene.

Aspects of the invention also provide a method of treating a cancer, autoimmune disorder, skin disease, inflammatory disease, ion channel disease, endocrine disease, extracellular matrix diseases, or metabolic disorder, said method comprising: (i) providing a population of cells obtained from a donor subject; (ii) introducing the expression vector disclosed herein to the cells; and (iii) introducing a nucleic acid guide complementary to the guide-binding sequence of the vector into the cells; (iv) wherein binding of the nucleic acid guide to the guide-binding sequence directs a mutation in a nucleic acid sequence of the vector resulting in expression of the polypeptide of interest in the cells; and (v) administering an effective amount of the cells to a recipient subject in need of treatment.

In some embodiments, the method further comprises introducing a gene editing tool into the cells. Preferably wherein the introduction of a gene editing tool is performed in (iii). In some embodiments, the gene editing tool is an endonuclease. In some embodiments, the endonuclease is selected from: a Zinc finger nuclease (ZFN), a Cas enzyme, or a transcription activator-like effector nucleases (TALENS), preferably wherein the enzyme or gene editing tool is a Cas enzyme, more preferably Cas9.

In some embodiments, the donor subject is the same as the recipient subject. In other words, the cells for administering to the recipient subject (iv) are autologous to the recipient subject.

In some embodiments, the cells are blood cells, stem cells, immune cells, or dermal cells. Preferably, the cells are T lymphocytes.

In some embodiments, the expression vector is integrated into the genomes of the cells.

In some embodiments, the mutation is a frameshift mutation at the guide-binding sequence. In some embodiments, the frameshift mutation is an insertion or deletion of a number of nucleotide residues not divisible by three. In some embodiments, the frameshift mutation is an insertion or deletion of one or two nucleotide residues, preferably an insertion of one nucleotide residue.

In some embodiments, the frameshift mutation at the guide-binding sequence shifts the coding sequence of the expression vector into frame with the start codon, such that the polypeptide of interest is expressed.

In some embodiments, the nucleic acid guide is 8 to 50 nucleic residues in length, preferably 15 to 35 nucleotide residues in length, more preferably 15 to 25 nucleotide residues in length.

In some embodiments, the nucleic acid guide comprises or consists of: SEQ ID NOs: 2-3, 23-26, 36-37 or 41 preferably SEQ ID NOs: 2, SEQ ID NO: 23 or SEQ ID NO: 25.

In some embodiments, the nucleic acid guide is located within a guide RNA. In some embodiments, the guide RNA comprises a guide RNA scaffold, preferably a guide RNA scaffold comprising SEQ ID NO: 4.

In some embodiments, the nucleic acid guide further binds to a guide-binding sequence in one or more endogenous genes and disrupts expression of the endogenous gene(s) in the cell. In some embodiments, the one or more endogenous gene(s) encode an immune checkpoint molecule. In some embodiments, the one or more endogenous genes are selected from the list comprising or consisting of: TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2 preferably wherein the endogenous genes are PD-1, TRBC1, TRBC2 and/or TRAC. In some embodiments, the nucleic acid sequence is an intragenic or intronic nucleic acid. In embodiments, the nucleic acid guides comprise or consist of a sequence complementary to a target sequence disclosed herein.

In some embodiments, binding of a nucleic acid guide to the guide-binding sequence in the expression vector initiates the same mutation as binding of the nucleic acid guide to the guide-binding sequence in an endogenous gene.

In some embodiments, the cancer is selected from the list comprising or consisting of: mesothelioma (e.g., malignant pleural mesothelioma); lung cancer (e.g., non-small cell lung cancer, small cell lung cancer, squamous cell lung cancer, or large cell lung cancer); pancreatic cancer (e.g., pancreatic ductal adenocarcinoma, or metastatic pancreatic ductal adenocarcinoma (PDA)); oesophageal adenocarcinoma, ovarian cancer (e.g., serous epithelial ovarian cancer), breast cancer, colorectal cancer, bladder cancer, haematological cancer, leukaemia or lymphoma, chronic lymphocytic leukaemia (CLL), mantle cell lymphoma (MCL), multiple myeloma, acute lymphoid leukaemia (ALL), Hodgkin lymphoma, B-cell acute lymphoid leukaemia (BALL), T-cell acute lymphoid leukaemia (TALL), small lymphocytic leukaemia (SLL), B cell prolymphocytic leukaemia, blastic plasmacytoid dendritic cell neoplasm, Burkitt's lymphoma, diffuse large B cell lymphoma (DLBCL), DLBCL associated with chronic inflammation, chronic myeloid leukaemia, myeloproliferative neoplasms, follicular lymphoma, paediatric follicular lymphoma, hairy cell leukaemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukaemia, splenic diffuse red pulp small B-cell lymphoma, hairy cell leukaemia-variant, lymphoplasmacytic lymphoma, a heavy chain disease, plasma cell myeloma, solitary plasmacytoma of bone, extraosseous plasmacytoma, nodal marginal zone lymphoma, paediatric nodal marginal zone lymphoma, primary cutaneous follicle centre lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukaemia (AML), or unclassifiable lymphoma.

In some embodiments, the autoimmune disorder is selected from the list comprising or consisting of: rheumatoid arthritis, psoriasis, arthritis, type 1 diabetes mellitus, lupus (including systemic lupus erythematosus), Addison's disease, celiac disease, dermatomyositis, Grave's disease, Hashimoto thyroiditis, multiple sclerosis, myasthenia gravis, pernicious anaemia, Sjögren's syndrome, autoimmune vasculitis, Guillain-Barre syndrome, vitiligo, chronic inflammatory demyelinating polyneuropathy, or sclerodoma, preferably rheumatoid arthritis, psoriasis, arthritis, type 1 diabetes mellitus, or lupus (including systemic lupus erythematosus).

In some embodiments, the metabolic disorder is selected from the list comprising or consisting of: familial hypercholesterolemia, malnutrition-inflammation-atherosclerosis syndrome, Gaucher disease, mucopolysaccharidosis type II (also known as Hunter syndrome), Krabbe's Leukodystrophy (also known as Krabbe's disease), stroke, or type 2 diabetes mellitus, maple syrup urine disease, metachromatic leukodystrophy, mitochondrial encephalopathy, lactic acidosis, stroke-like episodes (MELAS), Niemann-Pick, phenylketonuria, porphyria, Tay-Sachs disease, Medium-chain acyl-CoA dehydrogenase deficiency, galactosaemia, Glycogen storage disease (GSD) or Wilson's disease, preferably, malnutrition-inflammation-atherosclerosis syndrome, Gaucher disease, mucopolysaccharidosis type II (also known as Hunter syndrome), Krabbe's Leukodystrophy (also known as Krabbe's disease), stroke, or type 2 diabetes mellitus.

In some embodiments, the inflammatory disease is selected from the list comprising or consisting of: Alzheimer's disease, Parkinson's disease, fatty liver disease, endometriosis, type 2 diabetes mellitus, type 1 diabetes mellitus, inflammatory bowel disease, asthma, rheumatoid arthritis, ankylosing spondylitis, antiphospholipid antibody syndrome, gout, myositis, scleroderma, Sjogren's syndrome, systemic lupus erythematosus or vasculitis.

In some embodiments, the skin disease is selected from the list comprising or consisting of psoriasis, hives, vitiligo, or ichthyosis.

Aspects of the invention also provide an expression vector as disclosed herein, a combination as disclosed herein, or an isolated cell as disclosed herein, for use in treating a cancer, autoimmune disorder, skin disease, inflammatory disease, ion channel disease, endocrine disease, extracellular matrix diseases, or metabolic disorder.

### BRIEF DESCRIPTION OF THE DRAWINGS

The accompanying drawings are not intended to be drawn to scale. The Figures are illustrative only and are not required for enablement of the disclosure. For purposes of clarity, not every component may be labelled in every drawing.

FIG. 1. Selection of nucleic acid guide sequences for CRISPR.

FIG. 2. Current method for CAR integration and gene silencing. The steps for CAR integration and gene silencing are performed as separate, linear steps, and have diminishing returns.

FIG. 3. Coediting enables parallel purification of gene knockouts and CAR integration.

FIG. 4. Coediting proof of concept using CAR integration and four endogenous genes; TRAC (also referred to as TCRα), TRBC1 and TRBC2 (also referred to as TCRβ-1 and TCRβ-2) and PD-1. Using the present invention, endogenous genes can be switched off and genes of interest (such as a CAR) can be switched on in a single step using a single guide RNA (gRNA) and endonuclease.

FIG. 5. Schematic of a molecular switch cassette. A molecular switch cassette comprises at least a landing pad (purple), and a gene of interest (green) located downstream from the landing pad. The landing pad comprises a sequence complementary to a nucleic acid guide (referred to herein as the ‘guide binding sequence’) that directs a frameshift mutation to the landing pad. The landing pad and gene of interest are located downstream of a start codon (magenta). The gene of interest (green) is designed to be out of frame with the start codon (magenta) such that the gene of interest is not constitutively expressed. The gene of interest is designed to be out of frame with a start codon by the opposite amount of base pairs of the frameshift mutation in the guide binding sequence in the landing pad. For example, if the frameshift mutation is expected to be a +1 bp insertion, then the gene of interest is designed to be out of frame with a start codon by a −1 bp deletion. Therefore, when a nucleic acid guide directs a frameshift mutation to the guide binding sequence in the landing pad (i.e., when combined with a genetic editing tool such as a Cas enzyme), the frameshift mutation in the guide binding sequence landing pad causes the gene of interest to shift back into frame with the start codon, allowing the gene of interest to then be expressed. The molecular switch cassette may also comprise additional features, such as an SV40 NLS sequence (orange), a Peptide 2a (P2A) sequence (plum), a Kozak sequence (grey) and additional reporter genes, for example. The landing pad may also comprise a PAM sequence or complement thereof.

FIG. 6. Schematic of example molecular switch cassette before (A) and after (B) genetic editing. This figure shows an example of a molecular switch cassette where the guide binding sequence in the landing pad comprises a sequence complementary to a nucleic acid guide that targets the endogenous TRAC gene. In this example, the gene of interest is an ‘eGFP’ (green). The cassette is referred to herein as ‘TRAC-eGFP’. As shown in (A), the eGFP (green) is initially out of frame with the start codon (magenta), such that the eGFP is not constitutively expressed. A nucleic acid guide targeting the TRAC gene is expected to direct a +1 bp insertion to a complementary guide binding sequence in the landing pad. The eGFP has been designed to be out of frame with a start codon by a −1 bp deletion. Therefore, in the presence of the nucleic acid guide and a genetic editing tool (such as a Cas9 enzyme), the nucleic acid guide directs the genetic editing tool to produce a frameshift mutation in the guide binding sequence in the landing pad (shown as ‘N’ in (B)) which in turn shifts the eGFP back into frame with the start codon (magenta), allowing the eGFP to be expressed.

FIG. 7. Schematic of molecular switch cassette vector. Example of the ‘TRAC-eGFP’ molecular switch cassette inserted into a lentiviral vector. The vector may comprise additional features, such as a further reporter gene (e.g., mCherry as shown here), to indicate that the cassette has been successfully integrated into the target cells genome.

FIG. 8. FACS data showing expression of eGFP and mCherry after genetic editing of the TRAC-eGFP molecular switch cassette in HEK293 cells. ‘Non-switched ‘Off” shows the expression of mCherry without any genetic editing event (e.g., FIG. 6A). ‘Pre-switched ‘On” shows eGFP expression where eGFP has been placed in frame with a start codon. The ‘replicate’ panels show the results of three separate experiments where the molecular switch was genetically edited and switched on eGFP expression in HEK293 cells (e.g. as in FIG. 6B). TRAC is also referred to as TCRα.

FIG. 9. Reporter expression can be used to enrich for desirable DNA repair outcomes at the endogenous locus. Absence of reporter expression can be used to infer unintended endogenous DNA repair outcomes or mosaic repair outcomes.

FIG. 10. Example vectors comprising molecular switch cassettes where the gene of interest is (A) a chimeric antigen receptor (CAR; dark blue) with antigen binding domain against CD19 or (B) an HLA class I histocompatibility antigen, alpha chain E (HLA-E) (dark green). As with the expression vector of FIG. 6, the molecular switch cassette may be inserted into an expression vector, and may comprise comprises additional sequences to add the functionality of the invention, including a start codon (magenta), a PAM sequence or complement thereof (blue; to facilitate CRISPR editing), a P2A sequence (plum; to allow ribosomal skipping during translation to separate an earlier translated protein from the protein produced during translation of the gene of interest), an SV40 NLS sequence (orange; to allow targeting of the molecular switch cassette to the nucleus), one or more Kozak sequences (grey; a protein translation initiation site), one or more promoter (e.g., a CBh and/or a EF-1α), and a reporter gene downstream of the gene of interest (shown here as an ‘mCherry’ (red)). The guide binding sequence in the landing pad may be complementary to any suitable nucleic acid guide, such as a nucleic acid guide designed using the methods disclosed herein.

FIG. 11. Parallel validation of actionable IO targets.

FIG. 12. Molecular switch cassettes comprising landing pads with two guide binding sequences. Various conformations of landing pads were designed to test for the effect of directionality of the guide binding sequence as well as proximity when using two guide binding sequences. In A) to D) the first guide binding sequence is complementary to a nucleic acid guide that targets PD-1 and the second guide binding sequence is complementary to a nucleic acid guide that targets TRBC1/TRBC2. A) shows the ‘TT’ conformation where the first guide binding sequence is located on the reverse (3′ to 5′) strand followed by a PAM or complement thereof. Directly 5′ to the first guide binding sequence and PAM complement is a second guide binding sequence followed by a PAM or complement thereof. In this example, the guide binding sequences have a 5′ sequence complementary to a PAM, and the PAMs are located on the forward (5′ to 3′) strand. In other words, the guide binding sites (+PAM sequences or complements thereof) are located in series on the reverse (3′ to 5′) strand, directly one after another. B) shows the ‘TB’ conformation where the first guide binding sequence and PAM or complement thereof are located on the reverse (3′ to 5′) strand, and the second guide binding sequence and PAM or complement thereof are located on the forward (5′ to 3′) strand. In this example, the PAM for the first guide binding sequence is located on the forward (5′ to 3′) strand, and the PAM for the second guide binding sequence is located on the reverse (3′ to 5′) strand. C) shows the ‘TstuffT’ conformation where the first and second guide binding sequences (and PAM or the complements thereof) are located on the reverse (3′ to 5′) strand (as in (A)) but are separated by a 36 nucleotide ‘stuffer’ sequence (SEQ ID NO: 40). D) shows the ‘TstuffT’ conformation after a genetic editing has resulted in a deletion between the first and second guide binding sequence. E) shows the ‘Switch TstuffT’ conformation where the first and second guide binding sequence positions are switched so that the first guide binding sequence is complementary to a nucleic acid guide that targets TRBC and the second guide binding sequence is complementary to a nucleic acid guide that targets PD-1. In this embodiment the first and second guide binding sequences (along with their respective PAMs or complements thereof) are separated by a 36-nucleotide stuffer sequence (SEQ ID NO: 40).

FIG. 13. Molecular switch cassettes comprising landing pads with three guide binding sequences. Various conformations of landing pads were designed to test for the effect of directionality of the guide binding sequence as well as proximity when using three guide binding sequences. A) shows a ‘TTT’ conformation using three guide binding sequences where the first guide binding sequence is complementary to a nucleic acid guide that targets PD-1, the second guide binding sequence is complementary to a nucleic acid guide that targets TRBC, and the third guide binding sequence is complementary to a nucleic acid guide that targets TRAC. Here the first and second guide binding sequences and their PAMs or complements thereof are on the reverse (3′ to 5′) strand and are separated by a 36-nucleotide stuffer sequence comprising a stop codon (SEQ ID NO: 40). The third guide binding sequence and its PAM or complement thereof directly follow the second guide binding sequence on the reverse (3′ to 5′) strand. B) shows the ‘TTB’ conformation using three guide binding sequences where the first guide binding sequence is complementary to a nucleic acid guide that targets PD-1, the second guide binding sequence is complementary to a nucleic acid guide that targets TRBC, and the third guide binding sequence is complementary to a nucleic acid guide that targets TIM3. Here the first and second guide binding sequences and their PAMs or complements thereof are on the reverse (3′ to 5′) strand and are separated by a 36-nucleotide stuffer sequence comprising a stop codon (SEQ ID NO: 40). The third guide binding sequence and its PAM or complement thereof are placed on the forward (5′ to 3′) strand.

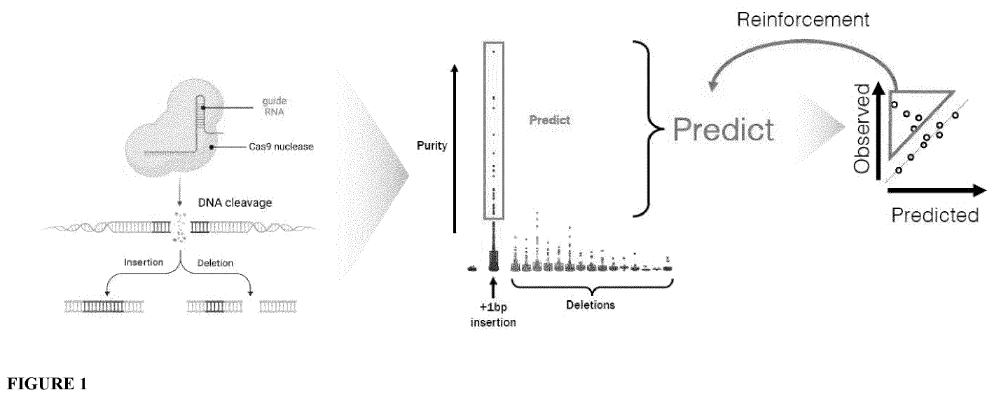

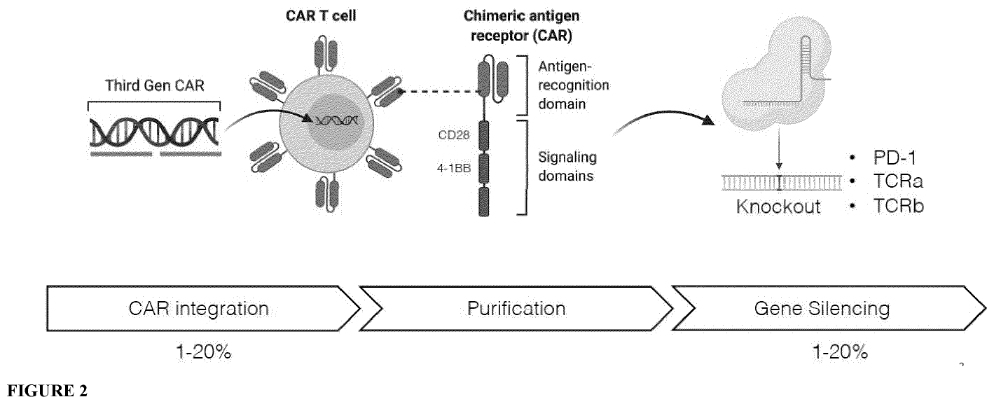

### DETAILED DESCRIPTION OF THE INVENTION

Unless otherwise defined below, all technical terms used herein have the same meaning as commonly understood by one of the ordinary skill in the art in the field to which this disclosure belongs.

### Definitions

Any reference to ‘or’ herein is intended to encompass ‘and/or’ unless otherwise stated.

As used herein, the singular forms ‘a’, ‘an’, and ‘the’ include both singular and plural referents unless the context dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. The term also encompasses ‘consisting of’ and ‘consisting essentially of’.

Whereas the term ‘one or more’, such as one or more members of a group of members, is clear per se, by means of further exemplification, the term encompasses inter alia a reference to any one of said members, or to any two or more of said members, such as, e.g., any ≥3, ≥4, 25, ≥6 or ≥7 etc. of said members, and up to all said members.

The term ‘nucleoside’ may refer to a molecule having a nucleobase (such as adenine (A), cytosine (C), guanine (G), thymine (T), or uracil (U)) covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as rare nucleosides).

‘Nucleotide’ or ‘nucleic acid residue’ or ‘nucleic acid’ may refer to a single nucleoside having one or more phosphate groups joined in ester linkages to the sugar moiety. A nucleotide comprising a nucleoside with a ribose sugar is a ‘ribonucleotide’ and commonly includes adenylate, cytidylate, guanylate, or uridylate. A nucleotide comprising a nucleoside with a deoxyribose sugar is a ‘deoxyribonucleotide’ and commonly includes deoxyadenylate, deoxycytidylate, deoxyguanylate or deoxythymidylate. ‘N’ used to denote any nucleotide. Unless specified otherwise or the context indicates otherwise (e.g. when referring to RNA guides), reference to nucleotides or nucleic acid residues as used herein may be understood to be referring to deoxyribonucleotides. Exemplary nucleotides include nucleoside monophosphates, diphosphates and triphosphates.

‘Polynucleotide’ or ‘nucleic acid’ may refer to a polymer of nucleotides joined together by a phosphodiester or phosphorothiorate linkage between 5′ and 3′ carbon atoms. A polynucleotide comprising ribonucleotides may be referred to as a ‘ribonucleic acid’ or ‘RNA’, and a polynucleotide comprising deoxyribonucleotides may be referred to as a ‘deoxyribonucleic acid’ or ‘DNA’.

‘Polypeptide’ or ‘protein’ may refer to a polymer of amino acids. One or more amino acid residues may be an artificial chemical analogue of a corresponding naturally occurring amino acid. The terms are also inclusive of modifications amino acids including, but not limited to, glycosylation, lipid attachment, sulfation, gamma-carboxylation of glutamic acid residues, hydroxylation and ADP-ribosylation.

‘Encoding a polypeptide of interest’ may refer to the ability of a nucleic acid sequence or amino acid sequence to result in the expression of a particular polypeptide.

A person skilled in the art will appreciate that the disclosed sequences may be modified to substitute one or more of the nucleotides or amino acids in the sequence for a nucleotide or peptide analogue or variant, respectively. Polynucleotides or polypeptides may be modified at any position so as to alter certain chemical properties of the polynucleotide or polypeptide yet retain the ability of the analogues or variants to perform their intended function. Analogues and variants have been described extensively in the art and are well known to a skilled person. The sequences disclosed herein are therefore intended to encompass obvious substitutions.

‘Homology’, ‘sequence identity’ or ‘sequence similarity’ in the context or two or more polynucleotides or polypeptides may refer to the extent to which the sequence of nucleotides or peptides are the same over a specified region. When comparing DNA and RNA, thymine (T) and uracil (U) may be considered equivalent. Therefore, a sequence that is ‘homologous’ to another sequence is the same as (or equivalent to) that sequence. The extent of homology may also be reported as a ‘percentage sequence similarity’, ‘percentage sequence identity’ or ‘percentage homology’, which may be calculated by aligning the two sequences, comparing the two sequences over the specified region, determining the number of positions at which identical nucleotides or peptides occurs in both sequences to yield the number of matched positions, dividing the number of matched positions by the total number of positions in the specified region, and multiplying the result by 100 to yield the percentage of sequence identity. Methods of aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet. Identity may be determined manually or by using a computer sequence algorithm such as ClustalW, ClustalX, BLAST, FASTA or Smith-Waterman. The popular multiple alignment program ClustalW (Nucleic Acids Research (1994) 22, 4673-4680; Nucleic Acids Research (1997), 24, 4876-4882) is a suitable way for generating multiple alignments of polypeptides or polynucleotides. Suitable parameters for ClustalW maybe as follows: For polynucleotide alignments: Gap Open Penalty=15.0, Gap Extension Penalty=6.66, and Matrix=Identity. For polypeptide alignments: Gap Open Penalty=10. o, Gap Extension Penalty=0.2, and Matrix=Gannet. For DNA and Protein alignments: ENDGAP=−1, and GAPDIST=4. Those skilled in the art will be aware that it may be necessary to vary these and other parameters for optimal sequence alignment. Suitably, calculation of percentage identities is then calculated from such an alignment as (N/T), where N is the number of positions at which the sequences share an identical residue, and T is the total number of positions compared including gaps but excluding overhangs. The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

‘Complement’ or ‘complementary’ may refer to the formation of hydrogen bonds between specific nucleobases (and therefore the nucleobase-containing nucleotides) to form double stranded DNA or RNA. This may also be referred to as ‘Watson-Crick’ and ‘Hoogsteen’ base pairing between nucleotides or nucleotide analogs. To do this, adenine is capable of forming a hydrogen bond with thymine for DNA or uracil for RNA, and guanine is capable of forming a hydrogen bond with cytosine in either DNA or RNA. Therefore, Adenine and Thymine/Uracil (A and T or U), and Guanine and Cytosine (G and C) may be referred to as ‘complementary’ nucleotides, respectively. Therefore, a complementary sequence is one where, when the nucleotides are aligned antiparallel to each other, the nucleotide bases at each position will be complementary. For example, if the target sequence is GTAC, then the complementary sequence in DNA would be CATG. Complementary sequences may be complementary over at least 5, 8, 10, 12, 15, 17, 20, 22, 25 or 30 nucleotides. In embodiments, the term complementary is used to refer to the reverse complementary sequence (i.e., complementary bases in reverse order). In this case, if the target sequence is CTTTA, then the reverse complementary sequence is TAAAG.

‘Complementary’ sequences can hybridize under low, middle, and/or high stringency condition(s).

The terms ‘bind’ or ‘hybridize’ may refer to the pairing of substantially complementary or complementary nucleic acid sequences within two different molecules. Pairing can be achieved by any process in which a nucleic acid sequence joins with a partially, substantially or fully complementary sequence through base pairing to form a hybridization complex. For purposes of hybridization, two nucleic acid sequences or segments of sequences are ‘substantially complementary’ if at least 80% of their individual bases are complementary to one another. Two nucleic acid sequences or segments of sequences are ‘partially complementary’ if at least 50% of their individual bases are complementary to one another. In embodiments, ‘binding’ refers to binding of a nucleic acid guide to a guide binding sequence.

The specificity of single-stranded DNA to hybridize complementary fragments is determined by the ‘stringency’ of the reaction conditions (Sambrook et al., Molecular Cloning and Laboratory Manual, Second Ed., Cold Spring Harbor (1989)). Hybridization stringency increases as the propensity to form DNA duplexes decreases. In polynucleotide hybridization reactions, the stringency can be chosen to favour specific hybridizations (high stringency), which can be used to identify, for example, full-length clones from a library. Less-specific hybridizations (low stringency) can be used to identify related, but not exact (homologous, but not identical), DNA molecules or segments. DNA duplexes are stabilised by: (1) the number of complementary base pairs; (2) the type of base pairs; (3) salt concentration (ionic strength) of the reaction mixture; (4) the temperature of the reaction; and (5) the presence of certain organic solvents, such as formamide, which decrease DNA duplex stability. In general, the longer the probe, the higher the temperature required for proper annealing. A common approach is to vary the temperature; higher relative temperatures result in more stringent reaction conditions. To hybridize under ‘stringent conditions’ describes hybridization protocols in which polynucleotides at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the given sequence hybridize to the given sequence at equilibrium. Since the given sequences are generally present at excess, at Tm, 50% of the probes are occupied at equilibrium.

‘Stringent hybridization conditions’ or ‘high stringency conditions’ are conditions that enable a probe, primer, or oligonucleotide to hybridize only to its specific sequence. Stringent conditions are sequence-5 dependent and will differ. Stringent conditions typically comprise: (1) low ionic strength and high temperature washes, for example 15 mM sodium chloride, 1.5 mM sodium citrate, 0.1% sodium dodecyl sulphate, at 50° C.; (2) a denaturing agent during hybridization, for example, 50% (v/v) formamide, 0.1% bovine serum albumin, 0.1% Ficoll, 0.1% polyvinylpyrrolidone, 50 mM sodium phosphate buffer (750 mM sodium chloride, 75 mM sodium citrate; pH 6.5), at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/mL), 0.1% SOS, and 10% dextran sulphate at 42° C., with a wash at 42° C. in 0.2×SSC (sodium chloride/sodium citrate) and 50% formamide at 55° C., followed by a high-stringency wash consisting of 0.1×SSC containing EDTA at 55° C. Suitably, the conditions are such that sequences at least about 65%, 70%, 75%, 85%, 90%, 95%, 98%, or 99% homologous to each other typically remain hybridized to each other.

‘Moderately stringent conditions’ or ‘moderate stringency conditions’ use washing solutions and hybridization conditions that are less stringent, such that a polynucleotide will hybridize to the entire, fragments, derivatives, or analogs of the polynucleotide. One example comprises hybridization in 6×SSC, 5×Denhardt's solution, 0.5% SOS and 100 μg/mL denatured salmon sperm DNA at 55° C., followed by one or more washes in 1×SSC, 0.1% SOS at 37° C. The temperature, ionic strength, etc., can be adjusted to accommodate experimental factors such as probe length. Other moderate stringency conditions have been described (see Ausubel et al., Current Protocols in Molecular Biology, Volumes 1-3, John Wiley & Sons, Inc., Hoboken, N.J. (1993); Kriegler, Gene Transfer and Expression: A Laboratory Manual, Stockton Press, New York, N.Y. (1990); Perbal, A Practical Guide to Molecular Cloning, 2nd edition, John Wiley & Sons, New York, N.Y. (1988)).

‘Low stringent conditions’ or ‘low stringency conditions’ use washing solutions and hybridization conditions that are less stringent than those for moderate stringency, such that a polynucleotide will hybridize to the entire, fragments, derivatives, or analogs of the polynucleotide. A non-limiting example of low stringency hybridization conditions includes 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other conditions of low stringency are well-described (see Ausubel et al., 1993; Kriegler, 1990).

The skilled person appreciates that there may be some tolerance in hybridisation of DNA and RNA sequences for non-canonical (i.e., non-complementary) base pairing. Therefore, a ‘complementary sequence’ with a specified percentage identity may be understood to encompass sequences capable of hybridizing, including sequences that may hybridize despite base pair mismatches or non-canonical base pairing.

The term ‘base pair’ or ‘bp’ may refer to a single, double-stranded pair of complementary DNA or RNA nucleotides. Therefore, when referring to a number of base pairs (e.g., 3 bp) in the present application, this may refer to the number of double-stranded nucleotides in a sequence.

‘Codon’ may refer to triplets of nucleotides that encode amino acids, start, or stop signals.

‘Stop codon’ may refer to a sequence of three nucleotides (i.e., a codon) in DNA or RNA that signals the termination of protein synthesis (i.e., translation) in a cell. A stop codon may be TAG, TAA or TGA in DNA (UAG, UAA or UGA in RNA, respectively). However, a person skilled in the art may use any suitable stop codon. ‘Alternative stop codons’ are codons that differ from those listed above and may be selected from the list comprising or consisting of: AGA, AGG, TCA or TTA in DNA (AGA, AGG, UCA or UUA in RNA, respectively).

‘Start codon’ may refer to a sequence of three nucleotides (i.e., a codon) in DNA or RNA codon, which will be the first codon translated into a polypeptide from the RNA. Therefore, the location of the start codon defines the polypeptide sequence that is translated and transcribed from a DNA or RNA sequence. A start codon may be ATG in DNA (AUG in RNA). However, a person skilled in the art may use any suitable start codon. ‘Alternative start codons’ that differ from the standard ATG (AUG) codon explained above and may be selected from the list comprising or consisting of: ATC, ATA, ATT, CTG, GTG, TTG, AAG, or AAG (AUC, AUA, AUU, CUG, GUG, UUG, AAG and AGG in RNA, respectively). All start codons code for methionine, as this is the first amino acid that is coded during protein synthesis. Even if alternative initiation codons are present, it eventually does get translated as methionine, even if the codon present normally does encode for a different amino acid. This happens because a separate tRNA is used for initiation in such cases.

‘Endogenous’ may refer to something, such as a polynucleotide or polypeptide, that originates from within an organism of interest. Therefore, ‘endogenous gene’ may refer to genes that originate from the genome of an organism of interest and are naturally occurring. In embodiments, the endogenous nucleic acid sequence is endogenous to the cell to which the present invention is being applied.

‘Exogenous’ may refer to something, such as a polynucleotide or polypeptide, that originates from out with an organism of interest.

As used herein, ‘downstream’ and ‘upstream’ may refer to the relative positioning of sequences in DNA or RNA. ‘Upstream’ may refer to a sequence that is closer to the 5′ end of the relevant DNA or RNA sequence than the comparative sequence. ‘Downstream’ may refer to a sequence that is closer to the 3′ end of the relevant DNA or RNA sequence than the comparative sequence.

‘Transcript’ or ‘primary transcript’ may refer to the single stranded RNA produced through transcription of DNA. ‘Primary transcript’ also encompasses precursor mRNA.

‘Genetic editing’ or ‘gene editing’ may refer to any modification of DNA, including insertion, deletion, modification, or insertion of nucleotides in a DNA sequence through techniques that are standard in the art. Insertions and deletions may be referred to as ‘indels’.

Insertions of nucleotides into a DNA sequence may be indicated by a ‘+’. Deletions of nucleotides from a DNA sequence may be indicated by a ‘-’. For example ‘+1 bp’ denotes a 1 base pair (double stranded nucleotide) insertion.

‘Editing outcome’ or ‘genetic editing outcome’ may refer to the result of a genetic editing event, i.e., +1 bp is a genetic editing outcome.

‘Gene editing tool’ or ‘genetic editing tool’ may refer to any standard method in the art through which genetic editing can be achieved. Preferably, the genetic editing tool is a nuclease. The genetic editing tool may be selected from the list comprising or consisting of: zinc-finger nucleases (ZFNs), transcription activator-like effector nucleases (TALENS), or Clustered Regularly Interspaced Short Palindromic Repeats (CRISPR).

As used herein, the terms ‘Cas protein’, ‘Cas effector’ or ‘Cas enzyme’ may refer to the CRISPR-associated (Cas) nucleases.

‘Zinc finger nuclease’ or ‘ZFN’ may refer to a chimeric polypeptide molecule comprising at least one zinc finger DNA binding domain effectively linked to at least one nuclease or part of a nuclease capable of cleaving DNA when fully assembled.

‘Transcription activator-like effector’ or ‘TALE’ may refer to a polypeptide structure that recognizes and binds to a particular DNA sequence. The ‘TALE DNA-binding domain’ may refer to a DNA-binding domain that includes an array of tandem 33-35 amino acid repeats, also known as RVD modules, each of which specifically recognizes a single base pair of DNA. RVD modules may be arranged in any order to assemble an array that recognizes a defined sequence. A binding specificity of a TALE DNA-binding domain is determined by the RVD array followed by a single truncated repeat of 20 amino acids. A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors, methyltransferases, integrases, nucleases, and recombinases. ‘Transcription activator-like effector nucleases’ or ‘TALENs’ may refer to engineered fusion polypeptides of the catalytic domain of a nuclease, such as endonuclease Fokl, and a designed TALE DNA-binding domain that may be targeted to a custom DNA sequence.

During translation and transcription of DNA to produce RNA and proteins, respectively, the DNA and RNA are read in a ‘reading frame’. The ‘reading frame’ divides the sequence of nucleotides in DNA or RNA into a set of consecutive, non-overlapping codons. The DNA or RNA sequence can therefore be read in multiple ways depending on which nucleotide the reading frame starts with. For example, a sequence of GATACTACA can be read starting from the first nucleotide (G) as GAT ACT ACA, starting from the second nucleotide (A) as ATA CTA CA or starting from the third nucleotide (T) as TAC A. This alters the grouping of the nucleotides into codons, and therefore changes the encoded amino acid, start or stop signals.

‘Mutation’ may refer to any change to nucleic acid residues within a sequence (such as insertions, deletions or changing one or more nucleic acid residues). In some embodiments, the mutation may be a frameshift mutation. ‘Frameshift mutation’ may refer to a genetic editing event that changes the reading frame for the sequence following the mutation site. As the DNA is read as codons (triplets of nucleotides), ‘frameshift mutation’ may refer to an insertion or deletion of a number of nucleic acid residues that is not divisible by three (i.e., insertion of 1, 2 or 4 nucleic acid residues, or the deletion of 1, 2 or 4 nucleic acid residues). A frameshift mutation can therefore result in a different grouping of nucleotides into codons and can therefore result in different amino acids being used to form the protein and/or the creation or removal of a premature stop codon. If the number of nucleic acid residues inserted or deleted in a nucleotide sequence is divisible by three, then the reading frame is unlikely to be changed and the inserted or deleted sequence only impacts the sequence of resulting protein. Preferably, the frameshift mutation may refer to the insertion of 1 nucleic acid residue. As used herein, +1 bp, +2 bp or +4 bp insertion or −1 bp, −2 bp or −4 bp deletion denotes the number of base pairs inserted, i.e., one nucleic acid residue in the sense strand, and one nucleic acid residue in the antisense strand.

‘Out of frame’ may refer to a DNA or RNA sequence that is frameshifted, i.e., it is not in the original reading frame, such that either no protein, a truncated protein, or an alternative protein is translated or transcribed from the DNA or RNA sequences.

‘In frame’ may refer to a DNA or RNA sequence, including mutated sequences, where the sequence is still in the correct reading frame, such that the majority of the original protein is still translated or transcribed from the DNA or RNA, and the resulting protein is partially or wholly functional. In cases where the DNA or RNA sequences are mutated, the resulting protein may be translated or transcribed with only some substitutions, modifications or missing sections.

‘Constitutively expressed’ may refer to a DNA sequence that is transcribed in an ongoing or continuous manner at the basal level.

‘Altering’ or ‘modulating’ expression may refer to a change in the expression of a polypeptide of interest from a DNA sequence, such as switching on or off the expression of a polypeptide of interest, or increasing or decreasing the expression of a polypeptide of interest. For example, a polypeptide of interest may not be constitutively expressed from a DNA sequence, but, after application of the products and/or methods described herein, the polypeptide of interest may be expressed. Alternatively, a polypeptide of interest may be constitutively expressed from a DNA sequence, but, after application of the products and/or methods described herein, the polypeptide of interest may no longer be expressed.

‘Nucleic acid guide’ may refer to a polymer of nucleic acids with a sequence that is complementary to a target sequence, preferably at least 5, 6, 7, 8, 9, 10, 11, 12, 13, 14, 15, 18, 20, 22, 25 or 30 nucleic acid residues of a target sequence, more preferably at least 5, 6, 7, 8, 9, 10, 11, 12, 13, 14, 15, 18, 20, 22, 25 or 30 consecutive nucleic acid residues of a target sequence. A nucleic acid sequence may be between 5 to 100, 5 to 80, 5 to 60, 5 to 50, 8 to 50, 8 to 40, 10 to 40, 10 to 35, 12 to 35, 14 to 35, 15 to 35 or 15 to 25 nucleic acid residues in length. The region of the target sequence that is complementary to a nucleic acid guide may be referred to as a guide binding sequence.

As used herein, ‘guide RNA’ or ‘gRNA’ may refer to an RNA molecule comprising a nucleic acid guide sequence and an RNA scaffold. When inserted into a guide RNA scaffold, the nucleic acid guide may be capable of directing a gene editing tool to produce a genetic editing event in the target DNA. It is understood by the skilled person that reference to a guide RNA in a DNA sequence may refer to a DNA sequence encoding the guide RNA.

‘Guide RNA scaffold’ may refer to a scaffold comprising standard nucleotide sequences configured to allow functionality of the guide RNA. In particular, the guide RNA scaffold allows interaction between the guide RNA with the gene editing tool and allows the nucleic acid guide to direct the gene editing tool to the target sequence. The scaffold may comprise or consist of a nucleotide sequence having at least 80, 81, 82, 83, 84, 85, 86, 87, 88, 89, 90, 91, 92, 93, 94, 95, 96, 97, 98, 99 or 100% sequence identity to SEQ ID NO: 4. It is understood by the skilled person that reference to the guide RNA scaffold in a DNA sequence may refer to a DNA sequence encoding the guide RNA scaffold.

‘Target sequence’ may refer to a DNA sequence intended for a genetic editing event. In embodiments, target sequence may refer to a DNA sequence comprising the guide binding sequence(s) and/or the complement thereof. In other words, target sequence may be used to refer to a double stranded region of DNA containing the guide binding sequences.

‘Guide binding sequence’ may refer to a nucleic acid sequence that is complementary to a nucleic acid guide sequence, such that the guide binding sequence and nucleic acid guide sequence may hybridise. In other words, the guide binding sequence is configured to hybridise to the nucleic acid guide sequence, including a nucleic acid guide sequence comprised within a guide RNA. The guide binding sequence may be located upstream of the coding nucleic acid sequence. The guide binding sequence may be a nucleic acid sequence located in a target sequence. Therefore, the region of the target sequence that is complementary to a nucleic acid guide may be referred to as a guide binding sequence.

‘Stuffer sequence’ may be used to refer to a nucleic acid sequence located between two guide binding sequences. In embodiments, the length of the stuffer sequence may not include the PAM sequence. When referring to a stuffer sequence between two guide binding sequences in a vector, it is preferable that the stuffer sequence does not influence function of the target cell. Therefore, the stuffer sequence may be any nucleic acid sequence that is non-functional.

As used herein, ‘landing pad’ may refer to a nucleic acid sequence comprising or consisting of one or more guide binding sequences. The landing pad may refer to a double stranded DNA comprising a guide binding sequence and a complement thereof. The landing pad may also further comprise one or more protospacer adjacent motif (PAM) sequences or a complement thereof. Preferably, the PAM is located in the DNA strand complementary to the strand containing the guide binding sequences. The landing pad is located upstream of the coding nucleic acid sequence.

‘Coding nucleic acid sequence’ or ‘coding sequence’ or ‘polynucleotide encoding’ may refer to a nucleic acid sequence encoding a protein of interest.

‘Protein of interest’ or ‘polypeptide of interest’ may refer to any protein which the experimenter desires to switch on expression of using the molecular switch cassette disclosed herein. In embodiments, the polypeptide of interest is a chimeric antigen receptor.

‘Expression’ may refer to the production of a functional product from a polynucleotide sequence. ‘Gene expression’ may encompass the stages of transcription, mRNA processing, non-coding RNA maturation, RNA export, translation, protein folding, translocation and protein transport. Therefore, expression may refer to any of the products of each of these stages. For example, expression of a polynucleotide may refer to transcription of the polynucleotide (for example, transcription resulting in mRNA or functional RNA) and/or translation of mRNA into a precursor or mature polypeptide. Preferably, ‘expression’ may refer to the production of a protein of interest from a polynucleotide sequence.

‘Functional’ may refer to a polypeptide that has biological function or activity.

‘Disruption’ of gene expression may refer to altering, reducing or preventing expression of the DNA, such that the resulting product is altered, reduced in quantity or prevented from being produced. In preferred embodiments, expression of the gene is reduced by at least 10%, 20%, 30%, 50%, 75%, 90% or 99% compared to the native gene.

‘Vector’ may refer to any vehicle that enables transport of any of the polynucleotides disclosed herein. ‘Expression vector’ may refer to a vector comprising any of the polynucleotides disclosed herein, with one or more further elements for enabling the expression of said polynucleotides in a cell. In embodiments, reference to the expression vector may refer to the vector once inserted into the genome of a host cell.

‘Cassette’ or ‘molecular switch cassette’ may refer to a nucleotide sequence comprising a guide binding sequence and a coding nucleic acid. The cassette may comprise a landing pad and a coding nucleic acid. The coding nucleic acid is located downstream of the guide binding sequence or landing pad. A cassette may also comprise additional regulatory elements or features, such as a promoter and/or a reporter gene. The cassette may be inserted into an expression vector. Unless otherwise specified, reference to the molecular switch cassette should be understood to refer to the cassette per se, i.e., the cassette when inserted into the expression vector, or the cassette after insertion into the genome of a host cell.

‘Promoter’ may refer to a synthetic or naturally-derived molecule which is capable of conferring, activating or enhancing expression of a polynucleotide in a cell. The term may refer to a polynucleotide element/sequence, typically positioned upstream and operably-linked to a polynucleotide, preferably a double stranded polynucleotide. Promoters can be derived entirely from regions proximate to a native gene of interest, or can be composed of different elements derived from different native promoters or synthetic polynucleotide segments. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents.

A ‘reporter gene’ encodes proteins that are readily detectable due to their biochemical characteristics, such as enzymatic activity, or chemifluorescent features.

The terms ‘introduced’, ‘provided’ or ‘applied’ means providing a polynucleotide (for example, a construct) or polypeptide into a cell. Introduced includes reference to the incorporation of a polynucleotide into a eukaryotic cell where the polynucleotide may be incorporated into the genome of the cell and includes reference to the transient provision of a polynucleotide or polypeptide to the cell. Introduced may refer to stable or transient transformation methods and may also refer to sexually crossing. Thus, ‘introduced’ in the context of inserting a polynucleotide (for example, a recombinant construct/expression construct) into a cell, means ‘transfection’ or ‘transformation’ or ‘transduction’ and may refer to the incorporation of a polynucleotide into a eukaryotic cell where the polynucleotide may be incorporated into the genome of the cell (for example, chromosome, plasmid, plastid or mitochondrial DNA), converted into an autonomous replicon, or transiently expressed (for example, transfected mRNA).

‘Transfection’ may refer to the introduction of DNA into a cell, particularly an animal cell or plant cell. Methods of transfection are standard in the art and may include electroporation, microinjection, biolistic particle delivery, magnetofection, lipofection, nanoparticles or the use of polymers or chemicals.

‘Transduction’ may refer to the introduction of DNA into a cell using viruses. Preferably, the method of transduction is lentiviral transduction.

‘Transformation’ may refer to the introduction of DNA into a cell through the cell membrane in bacterial cells or plant cells. Common methods of transformation include heat shock and electroporation.

‘Coediting’ as used herein may refer to a nucleic acid guide (such as a nucleic acid guide in a guide RNA) directing a genetic editing event at two or more loci. In the context of the present disclosure, coediting may refer to the same nucleic acid guide (such as a nucleic acid guide in a guide RNA) directing a genetic editing event to the landing pad of a molecular switch cassette, and one or more endogenous genes.

‘Concurrently’ may refer to coediting occurring using the same nucleic acid guide (such as a nucleic acid guide in a guide RNA) and gene editing tool (i.e., is performed in a single method step).

‘Simultaneously’ may refer to the introduction of nucleic acid guides at the same time (i.e., in a single step).

‘Donor subject’ may refer to an individual from which a population of cells are obtained. The donor subject may be a mammal, preferably a human.

‘Recipient subject’ may refer to an individual in need of treatment, to whom the cells of the present invention are applied. The recipient subject may be a mammal, preferably a human. In embodiments, the donor subject and the recipient subject are the same individual, i.e., the cells being received by the recipient subject are autologous.

‘Autologous’ may refer to cells obtains from the individual to be treated.

The terms ‘treating’ or ‘treatment’ as used herein refer to reducing the severity and/or frequency of symptoms, reducing the underlying pathological markers, eliminating symptoms and/or pathology, arresting the development or progression of symptoms and/or pathology, slowing the progression of symptoms and/or pathology, eliminating the symptoms and/or pathology, or improving or ameliorating pathology/damage already caused by the disease, condition or disorder.

‘Isolated cell’ or ‘ex vivo’ may refer to cells external to an organism. Methods referring to an isolated cell are not performed on a living organism.

### I. Nucleic Acid Guide

The nucleic acid guide may comprise or consist of a nucleic acid sequence with at least 80, 81, 82, 83, 84, 85, 86, 87, 88, 89, 90, 91, 92, 93, 94, 95, 96, 97, 98, 99 or 100% sequence identity to a region of a complementary target sequence, preferably a region between 5 and 100, 5 and 90, 5 and 80, 10 and 80, 10 and 70, 10 and 65, 10 and 60, 10 and 55, 10 and 50, 10 and 45, 10 and 40, 10 and 35 and 10 and 30 nucleic acid residues of the target sequence. The nucleic acid guide is capable of hybridising to the target sequence.

In embodiments, the target sequence is a guide binding sequence in an expression vector as described herein. In preferred embodiments, the target sequence is present both in an expression vector as described herein (i.e., as a guide binding sequence), and in one or more endogenous genes.

In embodiments, the target sequence is present in one or more genes endogenous to a mammal, more preferably, a gene endogenous to humans. In embodiments, the target sequence is an endogenous gene encoding an immune checkpoint molecule, preferably a gene selected from: TRAC (also referred to as TCR alpha chain constant), PD-1, CD38, CD39, TIM3, TIGIT, LAG3, TRBC1 (also referred to as TCRß-1), TRBC2 (also referred to as TCRβ-2), CISH, CD70, B2M, HLA-A, HLA-B, HLA-C, HLA-E, HLA-G, NKG2A, NKG2D, CBLB, TGFBR1, and TGFBR2, preferably wherein the endogenous genes are PD-1, TRBC1, TRBC2, and/or TRAC. In embodiments, the target sequence comprises or consists of a nucleic acid sequence with at least 80, 81, 82, 83, 84, 85, 86, 87, 88, 89, 90, 91, 92, 93, 94, 95, 96, 97, 98, 99 or 100% sequence identity SEQ ID NO: 1, SEQ ID NO: 20, SEQ ID NO: 21, SEQ ID NO: 35 or SEQ ID NO: 45. In preferred embodiments, the target sequence is a nucleic acid sequence with at least 80, 81, 82, 83, 84, 85, 86, 87, 88, 89, 90, 91, 92, 93, 94, 95, 96, 97, 98, 99 or 100% sequence identity to SEQ ID NOs: 13-15, 22, 27-30 or 42.

In embodiments, the nucleic acid guide may comprise or consist of a nucleic acid sequence having at least 80, 81, 82, 83, 84, 85, 86, 87, 88, 89, 90, 91, 92, 93, 94, 95, 96, 97, 98, 99 or 100% sequence identity to SEQ ID NOs: 2-3, 23-26, 36-37 or 41. In embodiments, the nucleic acid guide comprises or consists of: SEQ ID NOs: 2-3, 23-26, 36-37 or 41, preferably SEQ ID NOs: 2, 23, 25 or 41.

The nucleic acid guide may be between 5 to 100, 5 to 90, 5 to 80, 5 to 70, 5 to 60, 5 to 50, 8 to 50, 10 to 50, 10 to 45, 10 to 40, 10 to 35, 10 to 30, 12 to 40, 12 to 35, 12 to 30, 15 to 40, 15 to 35, or 15 to 25 nucleic acid residues in length.

The nucleic acid guide is capable of guiding a mutation at a complementary sequence, such as a guide binding sequence or an endogenous gene. In embodiments, the mutation is a frameshift mutation, preferably a frameshift mutation comprising or consisting of an insertion or deletion of a number of nucleic acid residues that is not divisible by three, such as an insertion or deletion of 1, 2 or 4 nucleic acid residues. In preferred embodiments, the frameshift mutation is the insertion of one or two nucleic acid residues, preferably an insertion of one nucleic acid residue. In embodiments, the frameshift mutation may disrupt expression of a target gene. In embodiment, the frameshift mutation may alter expression of a coding sequence, for example, when shifting a coding sequence back into frame with a start codon.

The nucleic acid guide may be designed using techniques that are standard in the art. For example, there are several, freely available software such as FOREcasT (Allen, Nature Biotechnology, Volume 37, Pages 64-72, 2019), inDelphi (Shen et al., Nature volume 563, page 646, 2018) or Lindel (Nucleic Acids Research, Volume 47, Pages 7989-8003, 2019). The FOREcasT model is available as a webtool (https://www.forecast.app) or can be run locally (e.g. using R programming language). The inDelphi model is also available via a webtool (available at https://indelphi.giffordlab.mit.edu/) or it can be run locally (e.g. in Python programming language). The Lindel model is also available as a webtool (https://lindel.gs.washington.edu/Lindel/docs/) or can be run locally (e.g. using Python programming language). Additionally, the Lindel model has been adapted into the CRISPOR guide design tool (available at https://www.crispor.tefor.net). Other suitable software include UCSC Genome Browser, and Deskgen.com. Methods of selecting suitable nucleic acid guides are also described in WO 2021/186163, which is incorporated by reference in its entirety.

A skilled person may also apply additional suitable controls, such as (i) selecting nucleic acid guides that target the first 50% of the gene, (ii) selecting nucleic acid guides with the highest value for the metric calculated from the fold change between the most abundant editing outcome and the second most abundant editing outcome, (iii) selecting nucleic acid guides based on their ranking for the metric ‘frameshift %’ (for example, using the Lindel model), (iv) selecting nucleic acid guides with an off-target score (for example, using Deskgen, UCSC Genome Browser and CRISPOR) of 70-100), (v) filtering out nucleic acid guides with undesirable on-target profiles (for example, using Deskgen which assigns a score of 0-100 based on the metric described by Doench et al., (Nature Biotechnology volume 34, pages 184-191 (2016), and the skilled person may filter out nucleic acid guides having scores of more than 35).

In one example, suitable nucleic acid guides may be identified through the following method:
1. The target DNA sequences can be identified using a publicly available genomics tool (e.g. ensemble.org);
2. All possible nucleic acid guide sequences that target the transcript of interest can be identified using publicly available software such as FOREcasT (Allen, Nature Biotechnology, Volume 64-72, 2019, available 37, Pages at https://www.forecast.app or can be run locally (e.g. using R programming language)), inDelphi (Shen et al., Nature volume 563, page 646, 2018, available at https://indelphi.giffordlab.mit.edu/or it can be run locally (e.g. in Python programming language)), or Lindel (Nucleic Acids Research, Volume 47, Pages 7989-8003, 2019, available at https://lindel.gs.washington.edu/Lindel/docs/or can be run locally (e.g. using Python programming language)). Additionally, the Lindel model has been adapted into the CRISPOR guide design tool (available at https://www.crispor.tefor.net). Other suitable software include UCSC Genome Browser, and Deskgen.com;
3. Nucleic acid guide sequences which targeted the second 50% of the gene can be filtered out;
4. Nucleic acid guide sequences can be ranked using the software described in #2. For example, nucleic acid guide sequences can be ranked in Lindel using the metric ‘frameshift %’. Nucleic acid guide sequences for which the major editing outcome was a multiple of three can be filtered out;
5. Nucleic acid guide sequences can be analysed to determine the fold change between the most abundant editing outcome and the second most abundant editing outcome using the software described in #2. The top 10 ranking Nucleic acid guide sequences can be selected;
6. Nucleic acid guide sequences with undesirable on-target profiles can be filtered out using the method described by Doench et al., (Nature Biotechnology volume 34, pages 184-191 (2016)), or using the software at ww.CRISPOR.tefor.net (described in #2). Nucleic acid guide sequences having scores of more than 35 (which have been found to work well in vitro and in vivo) can be selected; and
7. Nucleic acid guide sequences can be assigned an off-target score using the software. Suitable tools include UCSC Genome Browser and CRISPOR. The algorithm used by CRISPOR, along with most other tools is that of Hsu et al., (Nature Biotechnology volume 31, pages 827-832 (2013)). In the webtool the scores may range from 0 (many off targets) to 100 (no off targets). Nucleic acid guide sequences with a score of less than 70 can be filtered out;
8. Nucleic acid guide sequences can be sorted by frequency of a 1 bp insertion from highest (most chance of 1 bp insertion) to lowest. The top three nucleic acid guide sequences can then be selected for testing.

In embodiments, the nucleic acid guide may be inserted into a scaffold sequence, preferably a guide RNA scaffold sequence, even more preferably a single guide RNA. Suitable guide RNA scaffolds will depend on the choice of gene editing tool. Exemplary scaffold sequences will be evident to a person skilled in the art and are widely available from standard distributors in kits.

### II. Molecular Switch Cassette

A molecular switch cassette may comprise a guide binding sequence and a coding nucleic acid sequence, wherein the coding nucleic acid sequence is located downstream of the guide binding sequence.

In embodiments, the guide binding sequence may comprise or consist of a nucleic acid sequence with at least 80, 81, 82, 83, 84, 85, 86, 87, 88, 89, 90, 91, 92, 93, 94, 95, 96, 97, 98, 99 or 100% sequence identity to a sequence complementary to a nucleic acid guide, preferably a nucleic acid guide described herein. In embodiments, the guide binding sequence is capable of hybridising to a complementary nucleic acid guide, preferably a nucleic acid guide described herein. In embodiments, the guide binding sequence comprises or consists of a nucleic acid sequence with at least 80, 81, 82, 83, 84, 85, 86, 87, 88, 89, 90, 91, 92, 93, 94, 95, 96, 97, 98, 99 or 100% sequence identity to a target sequence disclosed herein, preferably between 5 and 100, 5 and 90, 5 and 80, 10 and 80, 10 and 70, 10 and 65, 10 and 60, 10 and 55, 10 and 50, 10 and 45, 10 and 40, 10 and 35, or 10 and 30 nucleic acid residues of a target sequence disclosed herein, preferably a target sequence comprising or consisting of SEQ ID NOs: 1, 20, 21, 35 or 45. In embodiments, the guide binding sequence may comprise or consist of a nucleic acid sequence having at least 80, 81, 82, 83, 84, 85, 86, 87, 88, 89, 90, 91, 92, 93, 94, 95, 96, 97, 98, 99 or 100% sequence identity to SEQ ID NOs: 13-15, 22, 27-30 or 42, preferably SEQ ID NOs: 13, 15, 27 or 42.

In embodiments, the molecular switch cassette may comprise two or more guide binding sequences. In embodiments, the guide binding sequences may comprise or consist of nucleic acid residues of different target sequences, preferably the target sequences disclosed herein.

In embodiments, the guide binding sequence may be between 5 to 100, 5 to 90, 5 to 80, 5 to 70, 5 to 60, 5 to 50, 8 to 50, 10 to 50, 10 to 45, 10 to 40, 10 to 35, 10 to 30, 12 to 40, 12 to 35, 12 to 30, 15 to 40, 15 to 35, or 15 to 25 nucleic acid residues in length.

In embodiments, the guide binding sequence does not comprise a premature or alternative STOP codon, particularly not in the sequence 5′ to the cleavage site. In embodiments, the target sequence does not comprise a premature or alternative STOP codon following the frameshift mutation.

In embodiments, the guide binding sequence is adjacent to a PAM. In embodiments, the guide binding sequence is upstream of a protospacer adjacent motif (PAM), or a complement thereof. In embodiments, the guide binding sequence is downstream (3′) to a sequence complementary to a PAM. The PAM may be located in the nucleotide strand complementary to the nucleotide strand that contains the guide binding sequence. For example, where the guide binding sequence is located on the 3′ to 5′ DNA strand, the PAM may be located on the 5′ to 3′ strand immediately 3′ to a sequence complementary to the guide binding sequence. In another example, where a guide binding sequence is located on the 5′ to 3′ DNA strand, the PAM may be located on the 3′ to 5′ strand immediately 3′ to a sequence complementary to the guide binding sequence. In other words, the PAM may be located downstream (3′) to a sequence complementary to the guide binding sequence.

In embodiments, the guide binding sequence is comprised within a landing pad, preferably wherein the landing pad further comprises a PAM or a complement thereof. In embodiments, the landing pad comprises two or more guide binding sequences. In embodiments, the landing pad comprises two or more guide binding sequences and two or more corresponding PAM sequences or complements thereof. The landing pad may be between 5 and 300, 5 and 250, 5 and 200, 10 and 300, 10 and 250, 10 and 200, 15 and 300, 15 and 250, 15 and 200, 15 and 150, 5 and 150, 10 and 150, 5 and 100, 5 to 90, 5 to 80, 10 to 90, 10 to 80, 10 to 70, 10 to 65, 10 to 60, 10 to 55, 10 to 50, or 10 to 45 nucleotides in length.

The skilled person will appreciate that any sequence encoding a protein of interest may be used as the coding nucleic acid. In embodiments, the coding nucleic acid is a chimeric antigen receptor (CAR).

CARs are standard in the art, and it is understood that the structure comprises or consist of four domains or regions; an antigen-recognition domain (or ligand binding domain), a hinge region, a transmembrane domain, and an intracellular signalling/activation domain (or endodomain). The antigen-recognition domain interacts with the target antigen. In embodiments, the antigen-recognition domain of the encoded CAR may comprise or consist of the variable region of monoclonal antibodies, or the antigen recognition domains of TNF receptors, innate immune receptors, cytokines, structural proteins and growth factors (Ahmad et al., 2022. Chimeric antigen receptor T cell structure, its manufacturing, and related toxicities; a comprehensive review. Advances in Cancer Biology—Metastasis, 4:100035). Methods for designing CARs are described in Guedan et al. (2019, Engineering and design of chimeric antigen receptors. Molecular Therapy Methods & Clinical Development, 12: P145-P156), Sadelain et al. (2013, The basic principles of chimeric antigen receptor (CAR)) and Kulemzin et al. (2017, Engineering chimeric antigen receptors. Acta Naturae, 9 (1): 6-14), which are incorporated by reference. In embodiments, the antigen may be HLA class I histocompatibility antigen alpha chain E (HLA-E), or HLA class I histocompatibility antigen alpha chain G (HLA-G), CD19, CD20, CD22, CD138, BCMA, CLL-1, PD-1, CD28, alpha-folate receptor, CD23, CD24, CD30, CD33, CD44v7/8, CEA, EGFRVIII, EGP-2, EGP-40, EphA2, erb-B2, erb-B3, erb-B4, FBP, fetal acetylcholine receptor, G_{D2}, G_{D3}, Her-2, HMW-MAA, IL-11Ralpha, IL-13R-alpha2, KDR, κ-light chain, Lewis Y, L1-cell adhesion molecule, MAGE-A1, Mesothelin, MUC1, MUC16, NKG2D ligands, NY-ESO-1 (157-165), Oncofetal antigen (h5T4), PSCA, PSMA, ROR-1, TAG-72, CD123, EGFR, GPC3, FAP, FRalpha, Igx, VEGFR, B7-H3 (CD276), B7H6 (NCR3LG1), CD5, CD70, CSPG4, EpCAM, HLA-A1, TAG72, 5T4, adenocarcinoma antigen, BAFF, B-lymphoma cell, C242 antigen, CA-125, carbonic anhydrase 9 (CA-IX), C-MET, CCR4, CD152, CD200, CD221, CD4, CD40, CD44 v6, CD51, CD52, CD56, CD74, CD80, CNTO888, CTLA-4, DRS, CD3, fibronectin extra domain-B, folate receptor 1, glycoprotein 75, GPNMB, HGF, human scatter factor receptor kinase, IGF-1 receptor, IGF-I, IgGI, LI-CAM, IL-13, IL-6, insulin-like growth factor I receptor, integrin a5β1, integrin avβ3, MORAb-009, MS4A1, mucin CanAg, N-glycolylneuraminic acid, NPC-1C, PDGF-R α, PDL192, phosphatidylserine, prostatic carcinoma cells, RANKL, RON, ROR1, SCH 900105, SDCI, SLAMF7, TAG-72, tenascin C, TGF beta 2, TGF-β, TRAIL-R1, TRAIL-R2, tumor antigen CTAA16.88, VEGF-A, VEGFR-1, VEGFR2 or vimentin. In preferred embodiments, the antigen is a HLA class I histocompatibility antigen alpha chain E (HLA-E).

In embodiments, the CAR may be a first, second or third generation CAR. In embodiments, the CAR may be a bi-specific CAR. When a protein (antigen) binds to the antigen recognition region (extracellular domain), there is transmission of an activation signal for the intracellular cell signalling domain, which in turn transmits this signal to the inside of the cell. The intracellular domain may transduce the effector function signal and direct the cell to perform its specialised function. Various intracellular cell signalling domains are known in the art. In embodiments, the intracellular signalling domain may be a CD-3 Zeta cytoplasmic domain, ζ chain of the T-cell receptor complex or any of its homologs (e.g., η chain, FcεR1γ and β chains, MB1 (Igα) chain, B29 (Igβ) chain, etc.), human CD3 zeta chain, CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

Costimulatory signals can be used to help CAR T cell proliferation, function, survival and antitumor activity. Costimulatory signals can be provided by incorporating intracellular signalling domains from one or more T cell costimulatory molecules into the CAR. In embodiments, the CAR comprises a co-stimulatory domain, preferably a co-stimulatory domain derived from the CD28 family (including CD28 and ICOS) or derived from the TNF receptor family (including TNFR-I, TNFR-II, 4-1BB, OX40, or CD27), CD134, Dap10, CD2, CD40L, TLRs, CD5, ICAM-1, LFA-1, Lck, Fas, CD30, or CD40, or combinations thereof. In embodiments, the co-stimulatory domain is located between the transmembrane domain and the intracellular domain (Weinkove et al. 2019. Selecting costimulatory domains for chimeric antigen receptors: functional and clinical considerations. Clin Transl Immunology, 8 (5): e1049, incorporated by reference).

Exemplary antigen receptors, including CARs, and methods for engineering and introducing such receptors into cells, include those described, for example, in international patent application publication numbers WO2014055668, WO200014257, WO2013126726, WO2012129514, WO2014031687, WO2013166321, WO2013071154, WO2013123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479,118, and European patent application number EP2537416, and those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75, Kochenderfer et al., 2013, Nature Reviews Clinical Oncology, 10, 267-276 (2013); Wang et al. (2012) J. Immunother. 35 (9): 689-701; and Brentjens et al., Sci Transl Med. 2013 5 (177).

In embodiments, the CAR has at least 80, 81, 82, 83, 84, 85, 86, 87, 88, 89, 90, 91, 92, 93, 94, 95, 96, 97, 98, 99 or 100% sequence identity to nucleotide positions 3256 to 4716 of SEQ ID NO: 17, or at least 80, 81, 82, 83, 84, 85, 86, 87, 88, 89, 90, 91, 92, 93, 94, 95, 96, 97, 98, 99 or 100% sequence identity to at least 500, 1000, 1100, 1200, 1300, 1400, or around 1460, nucleic acid residues between nucleotide positions 3256 to 4716 of SEQ ID NO: 17.

In embodiments, the CAR has at least 80, 81, 82, 83, 84, 85, 86, 87, 88, 89, 90, 91, 92, 93, 94, 95, 96, 97, 98, 99 or 100% sequence identity to nucleotide positions 3256 and 4329 of SEQ ID NO: 18, or at least 80, 81, 82, 83, 84, 85, 86, 87, 88, 89, 90, 91, 92, 93, 94, 95, 96, 97, 98, 99 or 100% sequence identity to at least 500, 600, 700, 800, 900, 1000, or around 1073, nucleic acid residues between nucleotide positions 3256 and 4329 of SEQ ID NO: 18.

In embodiments, the nucleic acid sequence encoding the protein of interest (i.e., the coding nucleic acid sequence) is out of frame with a start codon such that the protein of interest is not constitutively expressed. In embodiments, the coding nucleic acid sequence is out of frame with a start codon by a number of nucleic acids opposite to the number of nucleic acids expected to be inserted by a nucleic acid guide. For example, if the nucleic acid guide is expected to result in the insertion of 1 nucleic acid, the coding nucleic acid sequence will be out of frame by a deletion of 1 nucleic acid. In this way, when the nucleic acid guide directs a mutation to a sequence upstream of the coding nucleic acid sequence, for example, in the guide binding sequence, the mutation results in the coding sequence to be in frame with a start codon, resulting in expression of the polypeptide of interest.

### III. Vectors

There is provided an expression vector comprising the molecular switch cassette disclosed herein. In other words, there is an expression vector provided comprising a coding nucleic acid sequence encoding a polypeptide of interest, wherein the polypeptide of interest is not constitutively expressed from the vector; and a guide-binding sequence located upstream of the coding sequence, wherein the guide-binding sequence comprises a sequence complementary to a nucleic acid guide; wherein binding of a nucleic acid guide to the guide-binding sequence directs a mutation in a nucleic acid sequence of the vector resulting in expression of the polypeptide of interest.

It is understood that the term ‘expression vector’ refers to any vector capable of expressing a nucleic acid. Examples of expression vectors include plasmids, RNA expression vectors, viral vectors (including retroviral vectors, adenovirus vectors, poxvirus vectors, lentiviral vectors, herpesvirus vectors or adeno-associated virus vectors), or phage (bacteria) vectors. Viral vectors may be either replication competent or replication defective vectors.

In embodiments, the expression vector may comprise additional sequences to add to the functionality of the expression vector, including, but not limited to, a start codon, a P2A sequence (to allow ribosomal skipping during translation to separate an earlier translated protein from the protein produced during translation of the gene of interest), a nuclear localization signal (NSL; such as SV40 NLS, to allow targeting of the molecular switch cassette to the nucleus), one or more Kozak sequences (a protein translation initiation site), one or more promoters, and a reporter gene downstream of the gene of interest.

Examples of promoters include: EF-1α, CMV, CAG, EFS, CBh, CBA, SFFV, MSCV, SV40, hPGK, and UBC.

Examples of reporter genes include: but are not limited to: proteins that mediate antibiotic resistance (e.g., ampicillin resistance, neomycin resistance, G418 resistance, or puromycin resistance), coloured, fluorescent or luminescent proteins (e.g., green fluorescent protein or its derivatives (GFP), enhanced GFP (eGFP), red fluorescent protein or its derivatives (RFP), a blue fluorescent protein or its derivatives (EBFP, EBFP2, Azurite, mKalamal), monomeric Cherry (mCherry), tandem dimer Tomato (tdTomato), a yellow fluorescent protein or its derivatives (YFP, Citrine, Venus, YPet, EYFP), enhanced cyan fluorescent protein (ECFP, Cerulean, CyPet, mTurquoise2), UnaG, dsRed, eqFP61 1, Dronpa, TagRFPs, KFP, EosFP, Dendra, IrisFP, or luciferase) or enzymes (e.g. chloramphenicol acetyltransferase (CAT; Alton and Vapnek (1979) Nature 282:864-869), β-galactosidase (LacZ), β-glucuronidase, or alkaline phosphatase (Toh, et al. (1980) Eur. J. Biochem. 182:231-238; and Hall et al. (1983) J. Mol. Appl. Gen. 2:101). Reporter genes may also include detectable epitope tags such as one or more copies of the FLAG™, polyhistidine (His), myc, tandem affinity purification (TAP), or hemagglutinin (HA) tags or any detectable amino acid sequence. Reporter genes may be detected using techniques that are standard in the art, for example, fluorescence generated from fluorescent reporter genes can be detected with various commercially available fluorescent detection systems. Reporters may also be detected using standard biochemical techniques such as immunohistochemistry, or enzymes may generate a detectable signal when contacted with an appropriate substrate.

In embodiments, the expression vector is transfected into a host cell. Methods of transfection are standard in the art, and may include electroporation, microinjection, biolistic particle delivery, magnetofection, lipofection, nanoparticles or the use of polymers or chemicals.

The expression vector may exist transiently in a host cell or become integrated into the genome of a host cell. In preferred embodiments, the expression vector disclosed herein integrates into the genome of a host cell. Methods of integrating an expression vector into the genome of a host cell are standard in the art. For example, the skilled person is aware of methods of randomly integrating expression vectors into the genome, such as the use of lentivirus or transposase-based methods (such as piggyBac, Tol2 or Sleeping Beauty). Alternatively, by co-delivering a site-specific nuclease with a donor expression vector bearing homology arms (i.e., to the intended DNA site of insertion) can be integrated into a specific endogenous locus. Such nucleases include zinc finger nucleases, TALENs and CRISPR.

Assays detecting successful integration of an expression vector are standard in the art, and include, for example, polymerase chain reaction, sequencing assays, and restriction digestion assays.

In embodiments, the nucleic acid guide and the gene editing tool may be carried on a vector. In embodiments, the nucleic acid guide and the gene editing tool may be carried on the same vector. In preferred embodiments, the vector is a lentivirus or an AAV.

In embodiments, the host cell may be isolated from the host organism, i.e. ex vivo. In embodiments the host cell will be autologous to the intended recipient (i.e., the donor and recipient subject are the same). In embodiments the host cell may be a blood cell, a stem cell (preferably an adult stem cell), immune cell, or dermal cell, preferably a T cell or haematological stem cell.

### IV. Gene Editing Tools

In embodiments, the nucleic acid guide directs a gene editing tool to produce a mutation in a nucleic acid sequence in the expression vector, preferably the guide binding site. In embodiments, the nucleic acid guide also directs a gene editing tool to produce a mutation in an endogenous gene sequence.

In embodiments, the gene editing tool is an endonuclease, such as a Zinc finger nuclease (ZFN), a Cas enzyme (CRISPR system), or a transcription activator-like effector nuclease (TALENS).

CRISPR methods are standard in the art and are detailed in Doudna and Mali (2016. CRISPR-Cas a laboratory manual. Cold Spring Harbour Laboratory Press), which is incorporated by reference in its entirety. In brief, the CRISPR system comprises two mechanistic components, a nucleic acid guide sequence complementary to a target sequence, and a Cas endonuclease protein. Target recognition of target sequence by the nucleic acid guide is facilitated by the presence of a short motif called a protospacer-adjacent motif (PAM) in the target sequence or complement thereof, although some Cas proteins have been found to be ‘PAMless’. The guide RNA directs the Cas protein to cleave the target DNA to generate a single or double stranded break in the target DNA sequence (depending on which Cas protein is used). In embodiments, the Cas protein induces a single- or double-stranded break, preferably a double-stranded break in the target sequence.

The CRISPR/Cas systems are generally categorized into two classes (class I, class II), which are further subdivided into six types (type I-VI). Class I includes type I, III, and IV, and class II includes type II, V, and VI. Type I, II, and V systems recognize and cleave DNA, type VI can edit RNA, and type III edits both DNA and RNA. In embodiments, the gene editing tool is a type I, II, V or III Cas protein, preferably a type II Cas protein. In embodiments the Cas protein is a Cas9, preferably an SpCas9, SaCas9, NmCas9, CjCas9, StCas9, TdCas9, SpG, SpRY, xCas9, KKHSaCas9, ScCas9, or variant thereof. In preferred embodiments, the Cas9 is a an SpCas9 or variant thereof, preferably a TrueCutCas9 v2 (Invitrogen®).

A person skilled in the art appreciates that the PAM sequence depends on the Cas protein used. Numerous PAM sequences are known in the art. Exemplary PAM sequences and their compatible Cas proteins are listed in Table 11. N indicates any nucleic acid residue comprising cytosine, thymine, adenine or guanine or a derivative thereof, Y indicates any nucleic acid residue comprising cytosine, or thymine or a derivative thereof, R indicates any nucleic acid residue comprising adenine or guanine or a derivative thereof, and W indicates any nucleic acid residue comprising adenine or thymine or a derivative thereof.

TABLE 11
Exemplary Cas proteins and their compatible PAM sequences.

| Cas Protein | Suitable PAM Sequence(s) | Reference |
|---|---|---|
| SpCas9 | NGG | McDade, 2020. |
| | NGA | |
| SpCas9 D1135E | NGG | Kleinstiver et al. 2015a |
| SpCas9 VQR | NGAN | Kleinstiver et al. 2015a |
| | NGNG | |
| SpCas9 EQR | NGAG | Kleinstiver et al. 2015a |
| SpCas9 VRER | NGCG | Kleinstiver et al. 2015a |
| SaCas9 | NGRRT | McDade, 2020. |
| | NGRRN | |
| NmCas9 | NNNNGATT | McDade, 2020. |
| CjCas9 | NNNNRYAC | McDade, 2020. |
| StCas9 | NNAGAAW | McDade., 2020. |
| TdCas9 | NAAAAC | McDade., 2020. |
| SpG | NGN | Walton et al., 2020 |
| SpRY | NRN | Walton et al., 2020 |
| | NYN | |
| xCas9 | NG | Hu et al., 2018 |
| | GAA | |
| | GAT | |
| KKHSaCas9 | NNGRRT | Kleinstiver et al., 2015b |
| ScCas9 | NNG | Chatterjee et al., 2018 |
| ScCas9a | NNNGT | US 2019/0218532 |
| | NNGT | |

Chatterjee et al. (2018, Minimal PAM specificity of a highly similar SpCas9 ortholog, 4 (10): eaau0766).
Hu et al., (2018. Evolved Cas9 variants with broad PAM compatibility and high DNA specificity. Nature, 556 (7699): 57-63).
Kleinstiver et al. (2015a, Genome-wide specificities of CRISPR-Cas Cpf1 nucleases in human cells. Nature, 34:869-874).
Kleinstiver et al. (2015b, Broadening the targeting range of Staphylococcus aureus CRISPR-Cas9 by modifying PAM recognition, 33 (12): 1293-1298).
McDade et al. (2020, https://blog.addgene.org/the-pam-requirement-and-expanding-crisp-beyond-spcas 9).
Walton et al. (2020, Unconstrained genome targeting with near-PAMless engineered CRISPR-Cas9 variants. Science, 368 (6488): 290-296).

Therefore, in embodiments, the sequence complementary to the PAM may be selected from: CCN, TCN, NTCN, CNCN, CTCN, CGCN, NCN, NRN, NYN, CN, TTC, ATC, ARRCNN, NRRCN, AATCNNNN, GTYRNNNN, WTTCTNN, GTTTN, CNN, or CCNN, preferably wherein the sequence complementary to the PAM is CCN or TCN, wherein N is A, G, C or T, R is T or C, Y is a G or A, and W is A or T (recited 5′ to 3′).

If the present invention is used with a Cas protein as the gene editing tool, any scaffold sequence that comprises at least one stem loop structure and recruits an endonuclease may be used. Exemplary scaffold sequences for use with a Cas protein can be found, for example, in Jinek, et al. Science (2012) 337 (6096): 816-821, Ran, et al. Nature Protocols (2013) 8:2281-2308, WO 2014/093694, US 2014/0273226, WO 2013/176772 which are incorporated by reference. In preferred embodiments, the scaffold sequence comprises a trans-activating crRNA (also referred to as a ‘tracrRNA’) which serves as the binding region for an endonuclease (preferably a Cas9 protein). In embodiments, the tracr RNA region of the scaffold is between 30-50, 35 to 45, or around 42 nucleic acid residues in length. In embodiments, the tracrRNA and the nucleic acid guide are combined sequentially to form a guide RNA. A guide RNA has the dual function of both binding (hybridizing) to the target nucleic acid and recruiting the endonuclease to the target nucleic acid. In such embodiments, the nucleic acid guide may further comprise a linker loop sequence. Preferably, the guide RNA scaffold comprises or consists of SEQ ID NO: 4. In embodiments the nucleic acid guide is provided in a vector, preferably a lentivirus.

Zinc finger refers to a polypeptide structure that recognizes and binds to DNA sequences. A single zinc finger contains approximately 30 amino acids and the domain typically functions by binding 3 consecutive base pairs of DNA via interactions of a single amino acid side chain per base pair. A chain of zinc fingers may be used to recognise a longer, more specific sequence. Zinc finger nuclease or ZFN refers to a chimeric polypeptide molecule comprising at least one zinc finger DNA binding domain effectively linked to at least one nuclease or part of a nuclease capable of cleaving DNA when fully assembled, preferably the cleavage domain of the Fokl restriction enzyme. The Fokl domains dimerize for DNA cleavage and accomplish DSBs on targeted DNA sequences. Methods of using zinc finger nucleases to target specific nucleic acid sequences are detailed in Urnov et al., 2010. Genome editing with engineering zinc finger nucleases. Nature Reviews Genetics, 11:636-646, which is incorporated by reference in its entirety.

One method of gene editing may involve the use of transcription activator-like effector nucleases (TALENs) which induce double-strand breaks. TALENs are chimeric proteins that contain two functional domains: a DNA-recognition transcription activator-like effector (TALE) and a nuclease domain. The TALE comprises repeats of 33 or 34 amino acids with variations at amino acids 12 and 13 (referred to as the “Repeat Variable Diresidue”, or RVD) which mediate DNA binding. By changing the RVD sequence of a particular repeat, that repeat can be made to bind a specific nucleotide. The nuclease portion of a TALEN is the catalytically active domain of the restriction enzyme FokI, minus the DNA recognition domain. FokI can be used in mammalian cells to cut genomic DNA, but it must be dimerized to be functional. Therefore, a TALEN pair must bind on opposite sides of the target site, separated by a “spacer” ranging from 14-20 nucleotides. Methods of using TALENS are detailed in Joung and Sander, 2012. TALENS: a widely applicable technology for targeted genome editing. Nature Reviews Molecular Cell Biology, 14:49-55, which is incorporated by reference in its entirety.

Following a single- or double-stranded break in the target DNA, the host cell initiates repair mechanisms to repair the genome, which may be through the non-homology end joining (NHEJ) or high-fidelity homology directed recombination (HDR) pathways. The NHEJ and HDR pathways can introduce small insertions and deletions (indels) or result in the insertion of sequences, respectively. In embodiments, the break is repaired through the NHEJ pathway.

### IV. In Use

The nucleic acid guides disclosed herein can be used as a molecular switch to switch on expression of a protein of interest in an expression vector. In preferred embodiments, the nucleic acid guide can also be used to switch off expression of endogenous genes in a host cell. In preferred embodiments, the nucleic acid guide switches off endogenous genes and switches on expression of a gene of interest in a single step (i.e., substantially simultaneously or concurrently).

Following, or at the same time as delivery of the expression vector to the host cell, a nucleic acid guide and gene editing tool may be provided to the host cell, for example, using methods of transfection such as electroporation, microinjection, biolistic particle delivery, magnetofection, lipofection, nanoparticles or the use of polymers or chemicals.

In the cell, the nucleic acid guide directs the genetic editing tool to complementary sequences, such as the guide binding sequence in the molecular switch cassette, and/or one or more endogenous genes. In embodiments, the gene editing tool then produces a mutation, such as a frameshift mutation, in the complementary sequences. Where a frameshift mutation is produced there is a knock-on effect to the frame of the downstream nucleic acid sequence. As such, when a frameshift mutation is produced in the guide binding sequence of the molecular switch cassette, the frame of the downstream nucleic acid sequence encoding the protein of interest is also shifted by the number of base pairs inserted or deleted by the gene editing tool. The molecular switch cassette of the present invention is designed such that the nucleic acid sequence encoding the protein of interest is out of frame with a start codon with a number of base pairs opposite to that predicted to be inserted or deleted by the frameshift mutation (i.e., if the frameshift mutation in the guide binding sequence is expected to be an insertion of 1 nucleic acid residue, the nucleic acid sequence encoding the protein of interest is designed to be out of frame with a start codon by a deletion of 1 nucleic acid residue). In this way, the frameshift mutation in the guide binding sequence shifts the nucleic acid sequence encoding the protein of interest back into frame with a start codon, allowing the protein of interest to be expressed. As the nucleic acid guide is designed to also be complementary to a nucleic acid sequence in one or more endogenous genes, the nucleic acid guide may also direct the gene editing tool to produce a frameshift mutation in one or more endogenous gene sequences, resulting in disruption of the reading frame of the endogenous genes, and thereby disrupting expression of the endogenous genes. In this way, expression of a protein of interest may be switched on and expression of one or more endogenous genes may be switched off in a single method step using the same nucleic acid guide sequence (i.e., complementary to both the guide binding sequence and one or more endogenous sequences) when applied in combination with a gene editing tool.

Methods of detecting a frameshift mutation using standard techniques would be apparent to a person skilled in the art. For example, a skilled person may perform a PCR to amplify a region of the target sequence (in the one or more endogenous genes and/or the molecular switch cassette or expression vector), and then perform sequencing (for example, using Sanger sequencing or next generation sequencing) of the amplicon to detect changes to the nucleic acid sequence. The skilled person may also indirectly identify a frameshift mutation in the molecular switch cassette or expression vector by performing an assay to detect expression of the protein of interest and/or a reporter. This could include, for example, biochemical or imaging assays such as western blots, immunohistochemistry, ELISA, immunoassay, or flow cytometry.

### V. Methods of Treatment and Second Medical Use

In embodiments there is provided a method of treating a disease or disorder, using the expression vector and a nucleic acid guide as disclosed herein. In alternative embodiments, the expression vector, combination, isolated cell or population of cells disclosed herein is provided for use in treating a disease or disorder. In embodiments the disease or disorder is a cancer, autoimmune disorder, skin disease, inflammatory disease, ion channel disease, endocrine disease, extracellular matrix diseases, or metabolic disorder disclosed herein.

In embodiments, the cancer is selected from: lymphomas (such as diffuse large B cell lymphoma, primary mediastinal B cell lymphoma, Burkitt lymphoma, mantle cell lymphoma), and leukemias including lymphocytic and myeloid (such as acute myeloid leukemia (AML), chronic myeloid leukemia (CML), acute lymphocytic leukemia (ALL), chronic lymphocytic leukemia (CLL). In embodiments, the haematological disease is selected from: In embodiments the disease or disorder is β-thalassaemia or sickle-cell disease. In embodiments, the autoimmune disorder is selected from: Lupus (such as systemic lupus erythematosus) colitis, multiple sclerosis, graft-versus-host disease and type 1 diabetes. In embodiments, the inflammatory disease is selected from: diabetes type 1 and lupus. In embodiments, the neurological disease is a brain tumour. In embodiments, the metabolic disorder is selected from: diabetes and congenital hyperinsulinism.

As used herein, ‘treatment or prevention of a disease or disorder’ is referring to the use of the molecular switch cassette or expression vector, in combination with a nucleic acid guide described herein. More specifically, ‘treatment or prevention’ refers to use of the molecular switch cassette or expression vector, in combination with a nucleic acid guide described herein, to produce an isolated, modified cell, preferably a population of isolated, modified cells, for application to a subject in need of treatment.

In embodiments, the isolated cell, host cell or population of cells are isolated from a host organism, i.e., the cells are ex vivo. In alternative embodiments, the cells are in vivo, i.e. the expression vector may be administered to a subject in an in vivo treatment method, e.g. an in vivo gene therapy in which an exogenous polypeptide is expressed in the subject (and optionally expression of one or more endogenous genes in the subject is disrupted). In embodiments the host cell is autologous to the intended recipient (i.e., the donor and recipient subject are the same subject). In embodiments, the host cell is allogenic to the intended recipient (i.e., the donor and recipient subject are not the same subject). In embodiments the host cell may be a blood cell, a stem cell, immune cell, or dermal cell, preferably a peripheral blood mononuclear cell (PMBC), more preferably a T lymphocyte. In embodiments the T lymphocyte may be CD4+CD8− T cells, CD4− CD8+ T cells, naive T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In embodiments, the ‘population of cells’ from a donor subject comprises or consists of at least 10¹, 10², 10³, 10⁴, 10⁵, 10⁶, 10⁷, 10⁸, 10⁹, 10¹⁰, or 10¹¹ cells. In embodiments, the population of cells comprises or consists of between 10¹ and 10¹¹, 10² and 10¹¹, 10³ and 10¹¹, 10⁴ and 10¹¹, 10⁵ and 10¹¹, 10⁶ and 10¹¹, 10³ and 10¹⁰, 10⁴ and 10¹⁰, 10⁵ and 10¹⁰, 10⁶ and 10¹⁰, 10³ and 10¹⁹, 10⁴ and 10⁹, 10⁵ and 10⁹, 10⁶ and 10⁹ cells. In embodiments, the population of cells comprises or consists of 10¹, 10², 10³, 10⁴, 10⁵, 10⁶, 10⁷, 10⁸, 10⁹, 10¹⁰, or 10¹¹ white blood cells, preferably PMBCs and/or T lymphocytes. In embodiments, the population of cells comprises or consists of between 10¹ and 10¹¹, 10² and 10¹¹, 10³ and 10¹¹, 10⁴ and 10¹¹, 10⁵ and 10¹¹, 10⁶ and 10¹¹, 10³ and 10¹⁰, 10⁴ and 10¹⁰, 10⁵ and 10¹⁰, 10⁶ and 10¹⁰, 10³ and 10¹⁹, 10⁴ and 10⁹, 10⁵ and 10⁹, 10⁶ and 10⁹ white blood cells, preferably PMBCs and/or T lymphocytes.

In embodiments, the isolated cell, host cell or population of cells may be collected from the donor subject using any suitable extraction method in the art. For example, in embodiments where the isolated cell, host cell or population of cells are white blood cells, including T lymphocytes, the cells may be extracted from the donor subject using leukapheresis. During leukapheresis, blood is removed from the patient through a first intravenous line, the white blood cells are separated out from the blood, and the blood is then put back into the body through a second intravenous line.

Separation of the isolated cell, host cell or population of cells of interest from the remaining blood cells may be performed by apheresis, i.e., application of a centrifugal force to a continuous or semi continuous flow of anti-coagulated whole blood. In this process, white blood cells are located between the dense red blood cell layer and the less dense platelet/plasma layer. Different populations of white blood cells can then be separated out using methods that are standard in the art, such as washing and selection methods such as elutriation (i.e., the application of centrifugal force and counter-flow fluid to separate components based on size and density), antibody-bead conjugate selection and flow cytometry. Devices such as Haemonetics Cell Saver 5+, COBE2991, and Fresenius Kabi LOVO have the ability to remove gross red blood cells and platelet contaminants. Terumo Elutra and Biosafe Sepax systems provide size-based cell fractionation for the depletion of monocytes and the isolation of lymphocytes. Instruments such as CliniMACS Plus and Prodigy systems allow the enrichment of specific subsets of T cells, such as CD4+, CD8+, CD25+, or CD62L+ T cells using Miltenyi beads post-cell washing.

Alternatively, direct isolation of the isolated cell, host cell or population of cells from the blood may be achieved using technologies such as StraightFrom® microbeads, or RoboSep™.

In alternative embodiments, the isolated cell, host cell or population of cells are prepared from induced pluripotent stem cells (iPSCs), preferably, human iPSCs using standard techniques in the art.

In embodiments, the isolated cell, host cell or population of cells may be cultured to expand the number of cells using methods that are standard in the art, such as bioreactors. In embodiments, the cells may be cultured following transfection of the expression vector. Methods of culturing T cells for CART therapy are known in the art and are described in Wang and Riviere (2016. Clinical manufacturing of CAR T cells: foundation of a promising therapy. Mol Ther Oncolytics, 3:16015), which is incorporated by reference in its entirety.

In embodiments, the T cells may be activated. Activation of T cells may be achieved, for example, by using antigen-presenting cells (such as dendritic cells or artificial antigen-presenting cells (AAPCs)), bead-based activation (such as Invitrogen CTS Dynabeads CD3/28, Miltenyi MACS GMP ExpAct Treg beads, Miltenyi MACS GMP TransAct CD3/28 beads, and Juno Stage Expamer), antibody-coated magnetic beads or nanobeads, or anti-CD3 or anti-CD28 antibodies (such as OKT3). In embodiments, the T cells may be activated during ex vivo expansion. In embodiments, the T cells may be activated following introduction of an expression vector and/or nucleic acid guide and gene editing tool.

In embodiments, the isolated cell, host cell or population of cells may be treated to remove proliferation capability. In embodiments, the treatment for losing proliferation capability is irradiation (preferably gamma irradiation) or drug treatment.

Introduction of the expression vector and/or nucleic acid guide and gene editing tool may be through the use of vectors which may be transfected into the isolated cell, host cell or population of cells (e.g., using a transposon/transposase system such as the piggyBac, sleeping beauty, Frog Prince, Toll, or Tol2 systems), or transduced into the cell using o-retroviral vectors, lentiviral vectors. Methods of introducing expression vectors into cells, including T cells, are known in the art and are described in Wang and Riviere, 2016.

As used herein, the terms ‘administering’, ‘administer’ or ‘administration’ means providing to a subject or patient cells that have been modified using the method disclosed herein. The cells may be administered to the subject or patient using any suitable method of delivery. A preferred route of delivery is intravenous injection, but alternative delivery routes include intradermal, subcutaneous, intraperitoneal, intramuscular, intrathecal or direct injection into the brain, inhalation, rectal (suppository or retention enema), vaginal, oral (capsules, tablets, solutions or troches), transmucosal or transdermal (topical e.g., skin patches, opthalamic, intranasal) application.

As used herein, the term ‘effective amount’ refers to an amount of cells modified using the method described herein, which, when administered to a patient or subject with a disease or disorder, is sufficient to cause a qualitative or quantitative reduction in the severity or frequency of symptoms of that disease or disorder, and/or cause a qualitative or quantitative reduction in the underlying pathological markers or mechanisms. In embodiments, between 10¹ to 10¹⁰, 10² to 10¹⁰, 10³ to 10¹⁰, 10³ to 10⁹, 10³ to 10⁸, 10³ to 10⁷, 10³ to 10⁶, 10⁴ to 10¹⁰, 10⁴ to 10⁹, 10⁴ to 10⁸, 10⁴ to 10⁷ or 10⁵ to 10⁷ cells/kg are required for per single administration. The cells may be administered in a suitable carrier, diluent or excipient such as sterile water, physiological saline, glucose, dextrose, other buffer or the like. The cells may also be administered with additional therapeutic agents that are known in the art, such as stabilising agents, preservatives, antibiotics, vitamins, buffers, chelating agent, cytokines, growth factors or steroids. In embodiments, the effective amount of cells is sterilised. There is therefore provided a composition or comprising the cells treated using the method disclosed herein, preferably a composition comprising T lymphocytes treated using the method disclosed herein. In embodiments, there is provided a pharmaceutical composition comprising the cells comprising the cells treated using the method disclosed herein, preferably a pharmaceutical composition comprising T lymphocytes treated using the method disclosed herein, in combination with a second therapeutic agent, diluent, carrier or excipient.

In an embodiment, the effective amount of cells is administered only once. In a preferred embodiment, the effective amount of cells is administered multiple times. In one embodiment, a patient or subject is administered an initial dose, and one or more maintenance doses. Certain factors may influence the dosage required to effectively treat a subject or patient, including but not limited to the severity of the disease, disorder or condition, previous or concurrent treatments, the general health and/or age of the subject, and other diseases present. It will also be appreciated that the effective dosage of the cells for treatment may increase or decrease over the course of a particular treatment.

In an embodiment, the effective dose of cells may be administered in combination with other therapies for a related disease or disorder. In embodiments, the effective dose of cells is administered at the same time as other therapies for a related disease or disorder. In embodiments, the effective dose of cells is administered before or after other therapies for a related disease or disorder.

All publications mentioned in the above specification are herein incorporated by reference. Various modifications and variations of the described methods, uses and products of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

The invention will now be described by way of example only, with reference to the following non-limiting embodiments.

### Example 1: Designing and Testing Guide RNAs

### Methods

### Design of Nucleic Acid Guide Sequences

Nucleic acid guides predicted to direct a frameshift mutation (i.e., any insertion or deletion mutation that is not a multiple of 3) in a target DNA were designed using a modified version of the methods described in WO 2021/186163, although the skilled person may use any suitable method for designing nucleic acid guide sequences that are predicted to result in a frameshift mutation. In brief, the nucleic acid guides can be designed as follows:
1. The target DNA sequences can be identified using a publicly available genomics tool (e.g. ensemble.org);
2. All possible nucleic acid guide sequences that target the transcript of interest can be identified using publicly available software such as FOREcasT (Allen, Nature Biotechnology, Volume 37, Pages 64-72, 2019, available at https://www.forecast.app or can be run locally (e.g. using R programming language)), inDelphi (Shen et al., Nature volume 563, page 646, 2018, available at https://indelphi.giffordlab.mit.edu/or it can be run locally (e.g. in Python programming language)), or Lindel (Nucleic Acids Research, Volume 47, Pages 7989-8003, 2019, available at https://lindel.gs.washington.edu/Lindel/docs/or can be run locally (e.g. using Python programming language)). Additionally, the Lindel model has been adapted into the CRISPOR guide design tool (available at https://www.crispor.tefor.net). Other suitable software include UCSC Genome Browser, and Deskgen.com;
3. Nucleic acid guide sequences which targeted the second 50% of the gene can be filtered out;
4. Nucleic acid guide sequences can be ranked using the software described in #2. For example, nucleic acid guide sequences can be ranked in Lindel using the metric ‘frameshift %’. Nucleic acid guide sequences for which the major editing outcome was a multiple of three can be filtered out;
5. Nucleic acid guide sequences can be analysed to determine the fold change between the most abundant editing outcome and the second most abundant editing outcome using the software described in #2. The top 10 ranking Nucleic acid guide sequences can be selected;
6. Nucleic acid guide sequences with undesirable on-target profiles can be filtered out using the method described by Doench et al., (Nature Biotechnology volume 34, pages 184-191 (2016)), or using the software at www.CRISPOR.tefor.net (described in #2). Nucleic acid guide sequences having scores of more than 35 (which have been found to work well in vitro and in vivo) can be selected; and
7. Nucleic acid guide sequences can be assigned an off-target score using the software. Suitable tools include UCSC Genome Browser and CRISPOR. The algorithm used by CRISPOR, along with most other tools is that of Hsu et al., (Nature Biotechnology volume 31, pages 827-832 (2013)). In the webtool the scores may range from 0 (many off targets) to 100 (no off targets). Nucleic acid guide sequences with a score of less than 70 can be filtered out;
8. Nucleic acid guide sequences can be sorted by frequency of a 1 bp insertion from highest (most chance of 1 bp insertion) to lowest. The top three nucleic acid guide sequences can then be selected for testing.

We therefore designed nucleic acid guides that target one or more endogenous genes of interest; TRAC (ENST00000611116; SEQ ID NO: 1) PD-1 gene (ENST00000334409; SEQ ID NO: 20), TRBC1 (also referred to as TCRβ-1; ENST00000633705, SEQ ID NO: 33), and TRBC2 (also referred to as TCRβ-2; ENST00000466254; SEQ ID NO: 21). Although TRBC1 (TCRß-1) and TRBC2 (TCRß-2) are separate genes with separate transcripts, a nucleic acid guide that targeted both at the same time was designed. The selected nucleic acid guide sequences are shown in Table 1.

TABLE 1
Selected nucleic acid guide sequences.

| | Guide | | SEQ |
|---|---|---|---|
| Target Gene | No. | Sequence | ID NO: |
| TRAC | 1 | UUCGGAACCCAAUCACUGAC | 2 |
| | 2 | UGUGGUCCAGCUGAGGUGAG | 3 |
| | 7 | UUUCAAAACCUGUCAGUGAU | 36 |
| PD-1 | 3 | CAGCGGCACCUACCUCUGUG | 23 |
| | 4 | CUUCUGCCCUUCUCUCUGGA | 24 |
| | 8 | AGAACACAGGCACGGCUGAG | 37 |
| TRBC1/TRBC2 | 5 | UGGGAAGGAGGUGCACAGUG | 25 |
| (TCRß-1/TCRß-2) | 6 | AAAGGCCACACUGGUGUGCC | 26 |

### Preparation of Guide RNAs

Nucleic acid guides 1-8 (SEQ ID NOs: 2-3, 23-26 and 36-37, respectively) were inserted into a guide RNA scaffold, such as SEQ ID NO: 4, and ordered as 3 nmol modified synthetic single guide RNAs from Synthego. The lyophilized product was then resuspended in water upon receipt and stored at −80° C. until use.

### Electroporation of Cells of Interest

HEK293T cells were cultured as described in (Jiang et al. 2021, Protocol for cell preparation and gene delivery in HEK293T and C2C12 cells, STAR Protocols, 2 (3), 100497, https://doi.org/10.1016/j.xpro.2021.100497). On the day of electroporation, each of the guide RNAs were precomplexed with Cas9 (Invitrogen—TrueCutCas9 v2) by incubation at room temperature for 15 minutes with 5 μg Cas9 and 100 pmol of (guide RNA). HEK293T cells were then trypsinized and counted. For each electroporation, 200,000 cells were aliquoted into a 1.5 mL Eppendorf tube and centrifuged a 1000 rpm for 3 minutes, before removal of the supernatant. The remaining cell pellet was then resuspended in 20 μL of SF buffer (Lonza) mixed with Supplement 1 (Lonza; 82% SF buffer, 18% Supplement 1 as per manufacturer instructions). The cell mixture was then mixed with 2 μL of the precomplexed Cas9 and guide RNA. 20 μL of the cell/guide RNA/Cas9 mixture was then placed in a well of a 16 well cassette and placed into an Amaxa4D nucleofector with the X unit attachment (Lonza), and electroporate with Lonza program CM130. Cells were then added to cell culture medium (DMEM (Gibco), with 10% fetal bovine serum (FBS; Fisher Scientific)) and left to recover at 37° C./5% CO₂ for ten minutes before plating. The electroporated cells were then left to grow in at 37° C./5% CO₂ for three days.

### Genotyping

After three days, the electroporated cells were trypsinized and centrifuged as described above. The supernatant was removed and genomic DNA extracted from the remaining cell pellet using DNeasy Blood and Tissue kit (Qiagen) according to the manufacturer instructions. Polymerase chain reaction (PCR) was then used to amplify the region around the CRISPR cut site using Q5 polymerase (NEB) and the primers listed in Table 2, following the manufacturer protocol.

TABLE 2
Primers for PCR to identify gene editing in endogenous genes.

| Gene | Forward Primer | Reverse Primer | Tm* | Band Size |
|---|---|---|---|---|
| TRAC | TGAGCACCTACCCCATCCCC | AGTAGGAGAGTTTGGTGGGCT | 72 | 522 |
| | (SEQ ID NO: 5) | (SEQ ID NO: 6) | | |
| TRBC1 | ACCCTGGCTCCAACCCCTCT | TCATGGTGTGCGCTGGTTCCT | 72 | 541 |
| (TCRß-1) | (SEQ ID NO: 7) | (SEQ ID NO: 8) | | |
| TRBC2 | TGGTCCTTTCCCGGCCTTCT | ATCTCCCCAGGCCCCACTCA | 72 | 509 |
| (TCRß-2) | (SEQ ID NO: 38) | (SEQ ID NO: 39) | | |
| PD1 | TTGCTGCCCGAGGGATGTGAG | CTGATCCTGTGCAGGAGGGGAC | 72 | 521 |
| | (SEQ ID NO: 9) | (SEQ ID NO: 10) | | |

*Recommended melting temperature of the primers

### Calculating Genetic Editing Outcomes

The PCR product was electrophoresed on a 2% agarose gel to check whether the expected band size has been produced. After this quality control step, the PCR product was purified to remove polymerase, primers, and salts using the QIAquick PCR purification kit (Qiagen) and Sanger sequenced (Source Bioscience) using the forward primers listed in Table 2.

The Sanger sequencing data was processed using Synthego's Inference of CRISPR Edits (ICE) online tool (ice.synthego.com) to deconvolute endogenous editing events using machine learning.

The ‘ratio of single edits to all edits’ (herein referred to as ‘the editing ratio’) was then calculated by taking the percentage of single (intended) editing outcomes (i.e., a 1 base pair insertion, referred to as the ‘contribution’ value in the ICE tool) and dividing this value by the percentage of edited alleles (i.e., the proportion of total alleles where other editing events that were not intended, such as 2, 3, 4 bp insertions or deletions occurred, referred to as the ‘indel %’ in the ICE tool). Any guides where the editing ratio was over 80% can be used as a molecular switch.

### Results

Table 3 shows the percentage of edited alleles, percentage of single editing outcome, and the ratio of single edits to all edits for the tested guide RNAs. As seen from Table 3, both guides for TRAC could be used as a molecular switch under the criteria set out above. However, for the remaining experiments, the nucleic acid guide #1 (SEQ ID NO: 2) was used for targeting TRAC due to the higher ratio (86.5% compared to 81% with nucleic acid guide #2 (SEQ ID NO: 3)). For similar reasons, nucleic acid guides #3 and #5 (SEQ ID NOs: 23 and 25, respectively) were selected for further experiments to target PD-1, and TRBC1/TRBC2 (TCRβ-1/TCRβ-2) respectively.

TABLE 3
Genetic editing outcomes for selected nucleic acid
guides in HEK293T cells at the endogenous loci.

| | | | | | Ratio of |
|---|---|---|---|---|---|
| | | | | % Single | Single Edits |
| | Guide | SEQ ID | % Edited | Editing | to All |
| Target | No. | NO: | Alleles | Outcome | Edits (%) |
| TRAC | 1 | 2 | 93 | 80.5 | 86.5 |
| | 2 | 3 | 97.5 | 79 | 81 |
| | 7 | 36 | 47 | 34 | 72 |
| PD-1 | 3 | 23 | 99 | 99 | 100 |
| | 4 | 24 | 14.5 | 13.5 | 93 |
| TRBC1/T | 5 | 25 | 99 | 99 | 100 |
| RBC2 | 6 | 26 | 14 | 14 | 100 |
| (TCRβ-1 & | | | | | |
| TCRβ-2) | | | | | |

The selected guides were screened for premature stop codons and alternate start codons manually to ensure that these features do not impact the expression of the cassette of interest.

### Example 2: Designing a Molecular Switch Cassette

### Background

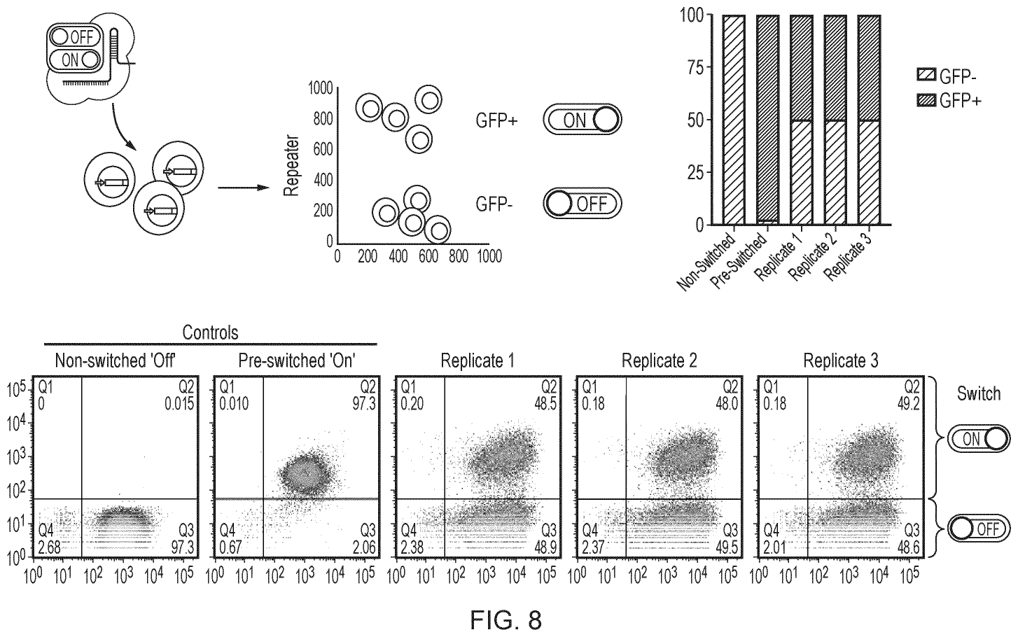

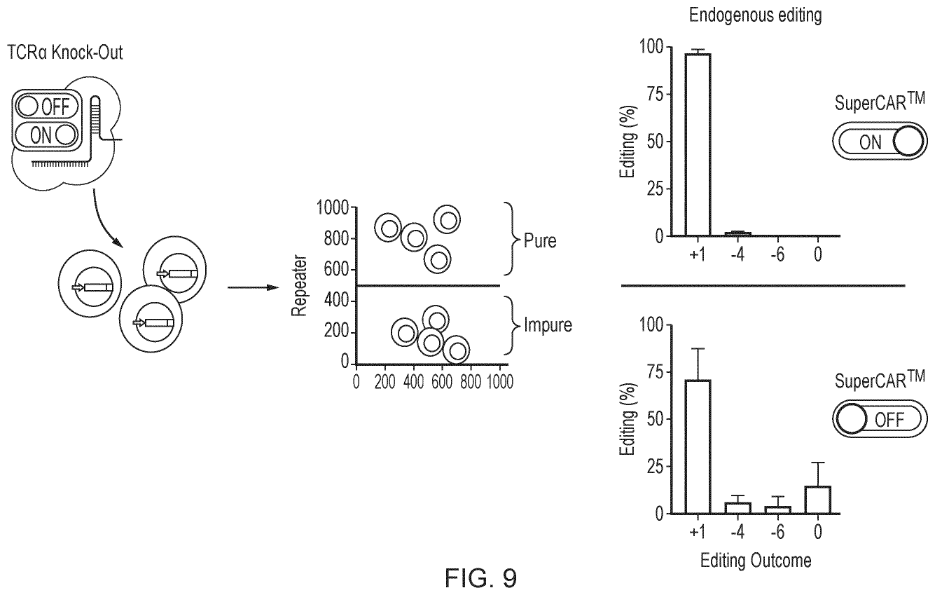

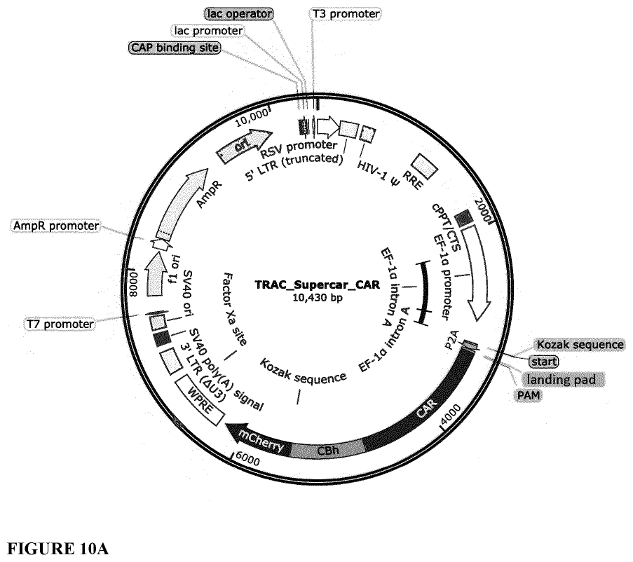

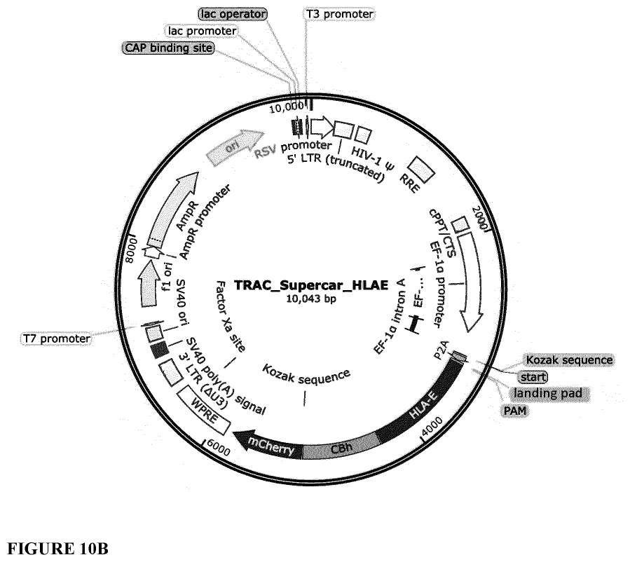

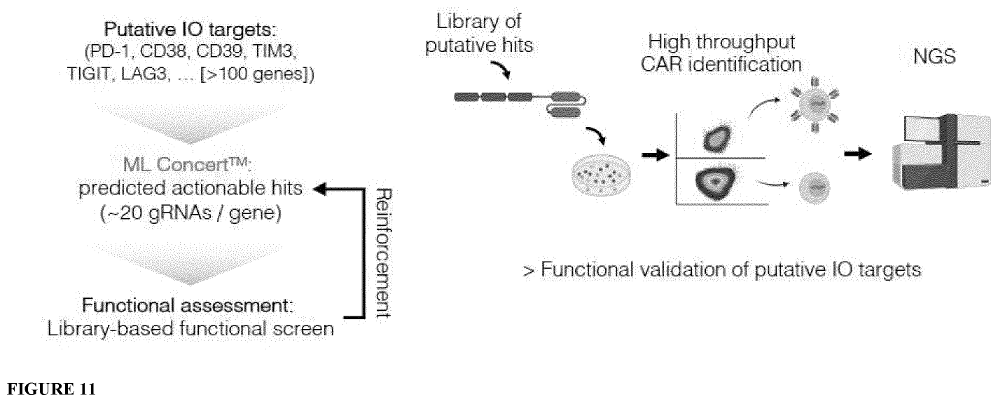

The nucleic acid guides designed as described in Example 1 can be used as a molecular switch; switching off endogenous genes and switching on expression of a gene of interest (i.e., a CAR) in a single step (FIG. 8).

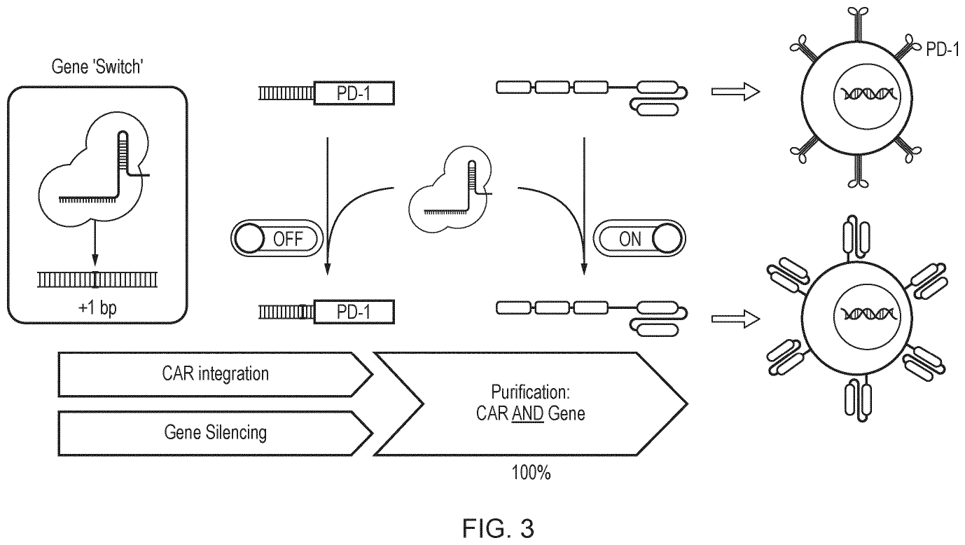

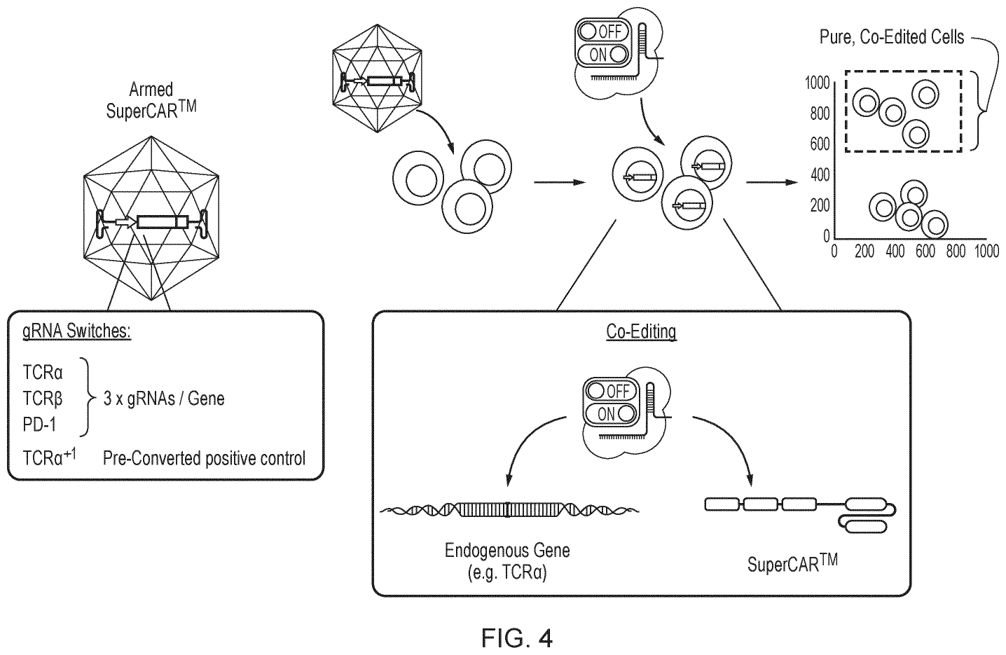

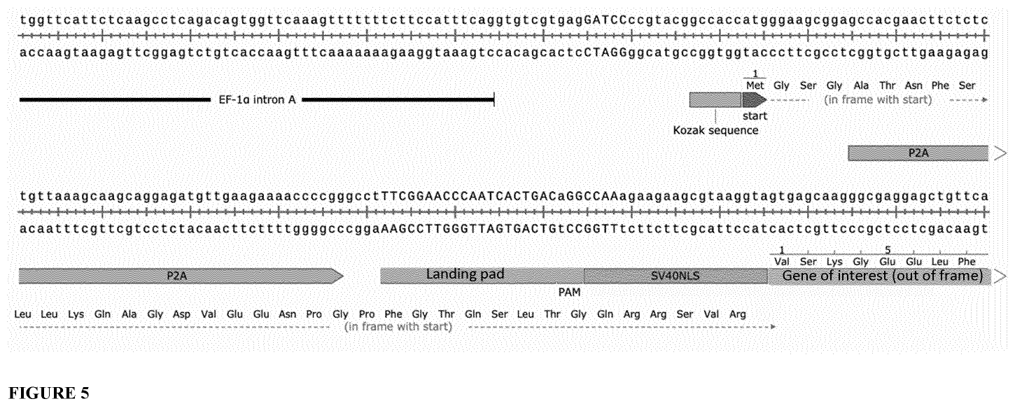

To switch on expression of a gene of interest, a molecular switch cassette was designed comprising a landing pad, and a gene of interest located downstream of the landing pad (FIG. 5). The landing pad comprises or consists of a sequence complementary to the nucleic acid guide sequences selected in Example 1. This complementary sequence in the molecular switch cassette is referred to herein as a guide-binding sequence. Importantly, the gene of interest was designed to be out of frame with a start codon by a number of base pairs opposite to the expected mutation directed by the nucleic acid guide of Example 1, such that the gene of interest is not normally expressed (FIG. 5). In other words, if the nucleic acid guide is expected to direct a +1 bp insertion, then the gene of interest was designed to be out of frame with a start codon by −1 bp. The molecular switch cassette can then be integrated into the genome of a target cell, and the nucleic acid guide and a genetic editing tool (such as a Cas enzyme) is provided to the cell through methods that are standard in the art. The nucleic acid guide directs the genetic editing tool to produce a frameshift mutation in the guide binding sequence in the landing pad of the molecular switch cassette (see FIGS. 3A and 3B). The frameshift mutation in the guide binding sequence is designed to have a knock-on effect of shifting the gene of interest back into frame with a start codon, allowing the gene of interest to be expressed. However, as the nucleic acid guide is designed to also be complementary to one or more endogenous genes, the nucleic acid guide may also direct a gene editing tool to endogenous sequences in the host cell.

### Methods

### Molecular Switch Cassette Design

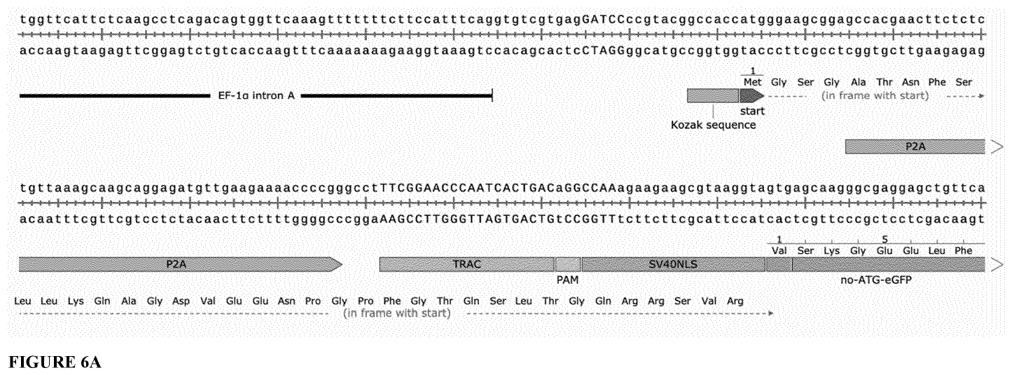

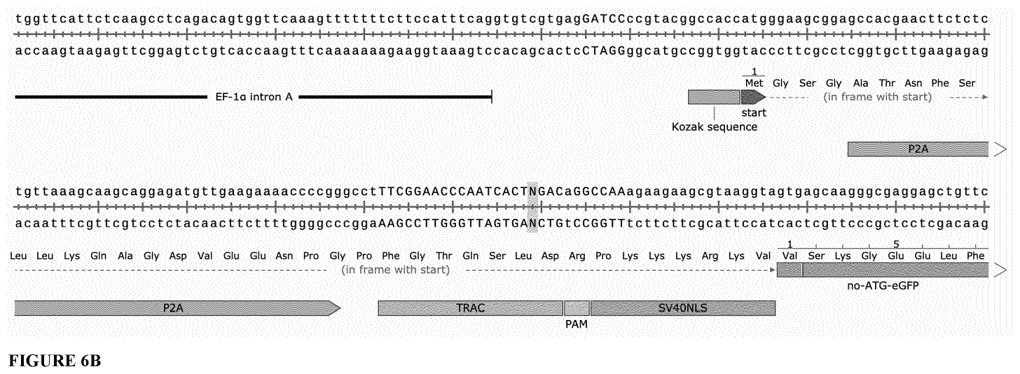

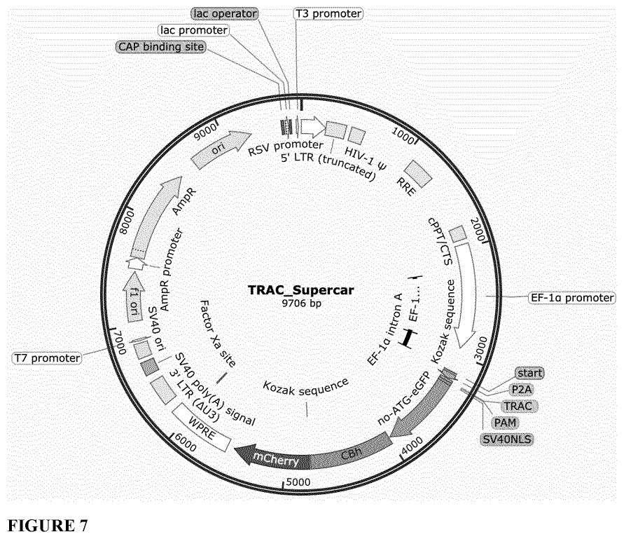

We designed molecular switch cassettes comprising a sequence complementary to each of the nucleic acid guide sequences selected in Example 1 (i.e., nucleic acid guides 1, 3, and 5 with SEQ ID NOs: 2, 23 and 25, respectively). This complementary sequence in the molecular switch cassette is referred to as the guide binding sequence. FIG. 6A shows an example of the cassette layout where the guide binding sequence is complementary to nucleic acid guide #1 (SEQ ID NO: 2, ‘TRAC’) and the gene of interest is an eGFP, located downstream of the landing pad. This cassette is herein referred to as the ‘TRAC-eGFP’ cassette. Molecular switch cassettes with guide binding sequences complementary to nucleic acid guides #3 and #5 were also produced with the same sequence features as the ‘TRAC-eGFP’ cassette, except the guide binding sequences (herein referred to as ‘PD-1-eGFP’ and ‘TRBC1 & TRBC2’ (or TCRβ-1 & TCRß-2), respectively). As can be seen from FIG. 6A, in the present experiments, the landing pads of the TRAC-eGFP, PD-1-eGFP and TRBC1/TRBC2 (TCRß-1/TCRß-2)-eGFP cassettes also comprised an NGG PAM complement 5′ to the guide binding sequence to facilitate Cas9 editing, and the molecular switch cassettes were designed to incorporate a start codon. As the selected nucleic acid guides directed a +1 bp insertion (see Example 1), the eGFP was designed to be out of frame with a start codon by −1 bp. As shown in FIG. 6A, the TRAC-eGFP, PD-1-eGFP and TRBC1/TRBC2 (TCRß-1/TCRß-2)-eGFP cassettes were designed to include a P2A sequence between the start codon and landing pad to allow ribosomal skipping during translation in order to separate an earlier translated protein from the gene of interest (i.e., eGFP). We also included an SV40 NLS sequence between the landing pad and eGFP to allow targeting of the molecular switch cassette to the nucleus of the cell to increase the chances of the molecular switch cassette integrating into the cell genome. The cassettes also comprised a promoter (EF-1α) and a Kozak sequence before the start codon to aid protein translation initiation.

### Lentiviral Construct Design and Production

It is preferable that the molecular switch cassette is integrated into the genome of the cells to allow stable transfection. A transient transfection of the cassette will be diluted due to the plasmid not being propagated during cell division. To integrate the molecular switch cassette into the genome of a target cell, we inserted the molecular switch cassette into a lentiviral construct.

FIG. 7 shows an example of the TRAC-eGFP molecular switch cassette inserted into a lentiviral vector (SEQ ID NO: 16). The PD-1-eGFP and TRBC1/TRBC2 (TCRβ-1/TCRß-2)-eGFP cassettes were also inserted into a lentiviral vector in the same way, and therefore all of the vectors had the same sequence except for the guide binding sequences. As can be seen in FIG. 7, the expression vectors comprised additional sequences including a constitutively expressed reporter gene (mCherry) to allow detection of the construct driven under a CBh promoter. The constitutively expressed mCherry is unaffected by the frameshift mutation due to being expressed under a second promoter and kozak sequence.

The lentiviral backbone was purchased from Vector builder using their standard ‘mammalian gene lentivirus expression vector’. The EF1a, eGFP, CBH, mCherry portions were cloned in using standard molecular biology techniques (Gibson assembly). To produce the lentiviruses, the landing pad was cloned into the BsmBI cloning site.

The lentivirus was produced using HEK293T cells. In brief, the HEK293T cells were seeded into 10 cm dishes and after 24 hours of culture, the cells were transfected with the molecular switch cassette plasmid, the packaging plasmid (PsPax; Addgene #12260), and the envelope plasmid (PDM2G; Addgene #12259) using Lipofectamine 3000 according to the manufacturer instructions. The media was collected from the cells 48 hrs after transfection, centrifuged and filtered through a 0.45 um filter. This contained the virus and was stored for later use.

Fresh HEK293T cells were seeded onto 6 well plates to titre the virus using polybrene to facilitate infection. After 4 days, mCherry could be observed under the microscope. The cells underwent flow cytometry at day 4 post infection to select for the population of cells expressing mCherry (i.e., transfected cells containing the construct). Flow cytometry was performed as described in Rico et al. (2021. Flow-cytometry-based protocols for human blood/marrow immunophenotyping with minimal sample perturbation. STAR Protocols, 2 (4): 100883, https://doi.org/10.1016/j.xpro.2021.100883). mCherry positive cells were cultured for one week.

### Testing the Molecular Switch

Nucleic acid guides were ordered in guide RNA scaffolds as outlined in Example 1. Each guide RNA was individually pre-complexed with Cas9 (Invitrogen—TrueCutCas9 v2) at room temperature for 15 minutes with 5 μg Cas9 and 100 pmol guide RNA before electroporation as described in Example 1.

mCherry-expressing HEK293T Cells were trypsinized and counted. For each electroporation, 200,000 cells were aliquoted into a 1.5 mL Eppendorf tube and centrifuged a 1000 rpm for 3 minutes, before removal of the supernatant. The remaining cell pellet was then resuspended in 20 μL of SF buffer (Lonza) mixed with Supplement 1 (Lonza; 82% SF buffer, 18% Supplement 1 as per manufacturer instructions). The cell mixture was then mixed with 2 μL of the precomplexed Cas9 and guide RNA. 20 μL of the cell/guide RNA/Cas9 mixture was then transferred to a well of a 16 well cassette and placed into an Amaxa4D nucleofector with the X unit attachment (Lonza), and electroporate with Lonza program CM130. Cells were then added to cell culture medium (described in Example 1) and left to recover at 37° C./5% CO₂ for ten minutes before plating on a 6 well plate. The electroporated cells were then left to grow in at 37° C./5% CO₂ for three days. Electroporations were run in triplicate for each cassette.

After three days, green cells were observed under the microscope. The cells were then trypsinized, run through flow cytometry and cells sorted into eGFP+ and mCherry+ populations. The eGFP+ and mCherry populations were genotyped separately, following the protocol detailed under Example 1 ‘Genotyping’ to detect genetic editing events in the endogenous genes.

To test for editing of the molecular switch cassette, the same genotyping protocol was used as in Example 1 ‘Genotyping’, but the primers listed in Table 4 were used instead of the primers listed in Example 1. The primers listed in Table 4 are suitable for the molecular switch cassette described herein regardless of the landing pad sequence used.

TABLE 4
Primers for genotyping the molecular switch cassette

| Target | Forward Primer | Reverse Primer | Tm | Band Size |
|---|---|---|---|---|
| Cassette | TCAGGTGTCGTGAGGATCC | CATAAGGTCATGTACTGGGCAC | 66 | 1131 |
| | (SEQ ID NO: 11) | (SEQ ID NO: 12) | | |

The percentage of gene editing events for the endogenous loci and the cassette were calculated using the ICE webtool for Sanger sequencing deconvolution (https://www.synthego.com/products/bioinformatics/crispr-analysis).

### Results

The percentage of gene editing events for the endogenous loci and the cassette are shown in Table 5. Co-editing is considered a success when the editing events at the molecular switch cassette and the endogenous locus match. Table 5 demonstrates that the present invention allowed successful co-editing of the endogenous genes TRAC, TRBC1/TRBC2 (TCRβ-1/TCRβ-2) or PD-1 and the eGFP molecular switch cassette.

TABLE 5
Evaluation of co-editing of endogenous gene and the cassette.

| | | | TRBC1 | TRBC2 | |
|---|---|---|---|---|---|
| | TRAC | | (TCRβ-1) | (TCRβ-2) | PD-1 |
| eGFP | +1* | (97%) | +1 (99%) | +1 (99%) | +1 (99%) |
| Cassette | −4 | (2%) | | | |
| Endogenous | +1 | (98%) | +1 (99%) | +1 (99%) | +1 (99%) |
| Locus | −4 | (2%) | | | |

*+1/−4 indicates the detected mutation and percent of detected alleles with that mutation.

FIG. 8 demonstrates mCherry and eGFP expression when using the ‘TRAC-eGFP’ cassette and nucleic acid guide #1 (SEQ ID NO: 2). As shown in FIG. 8, without any genetic editing event (‘Off’; e.g. as in FIG. 6A), there is no expression of eGFP (upper panel), but there is constitutive expression of the reporter mCherry (lower panel). ‘Pre-switched ‘On” simply may refer to a positive control without any genetic editing where the eGFP is in frame with a start codon. The ‘replicate’ panels show the results of three separate experiments where the TRAC-eGFP cassette expression vector was transfected into the HEK293T cells and genetically edited using Cas9 and the TRAC nucleic acid guide #1 (SEQ ID NO: 2; e.g., as in FIG. 6B). FIG. 8 therefore shows that the molecular switch method consistently resulted in eGFP expression in the HEK293 cells.

Obviously, the cassette is not limited to a fluorescent protein and can consist of any gene of interest, including, but not limited to, chimeric antigen receptors (such as the expression vector shown in FIG. 6A). Any suitable nucleic acid guide designed as in Example 1, may be used, in combination with any suitable genetic editing tool, not just Cas9 as used here.

### Example 3: Effect of Multiplexing and Altering Orientation of Guide RNAs on Coediting

### Background

Multiple genes of interest can be simultaneously co-edited whilst switching on expression of a gene of interest in a molecular switch cassette by using more than one guide binding sites in series (referred to herein as multiplexing). In this case, the molecular switch cassette is designed such that the gene of interest is out of frame with a start codon by a number of base pairs corresponding to the total number of inserted or deleted base pairs by genetic editing events at all of the guide binding sites. Therefore, an editing event at all guide binding sites is required for the gene of interest be shifted back into frame with a start codon and express the gene of interest and a single editing event would be insufficient to cause expression.

### Methods

### Design of Nucleic Acid Guides for Multiplexing

Nucleic acid guides are designed as described in Example 1.

### Molecular Switch Cassette Design

Molecular switch cassettes and lentiviral constructs (where the gene of interest is an eGFP) were designed as described in Example 2 with one exception; the landing pads in the present example comprised two or three guide binding sequences in a series. Each of the guide binding sequences were located on either the forward strand or the reverse strand.

When using guide binding sites in the molecular switch cassette which result in a number of base pair insertions or deletions divisible by three, a stop codon was inserted between each of the guide binding sites to allow a frameshift to occur.

Various conformations of landing pads were designed to test for the effect of directionality of the guide binding sequence as well as proximity when using two or more guide binding sequences. These conformations were tested using guide binding sequences complementary to nucleic acid guides #3 (SEQ ID NO: 23) and #5 (SEQ ID NO: 25) against PD-1 and TRBC, respectively (i.e., guide binding sequences with SEQ ID NOs: 15 and 27) and are summarised in Table 6.

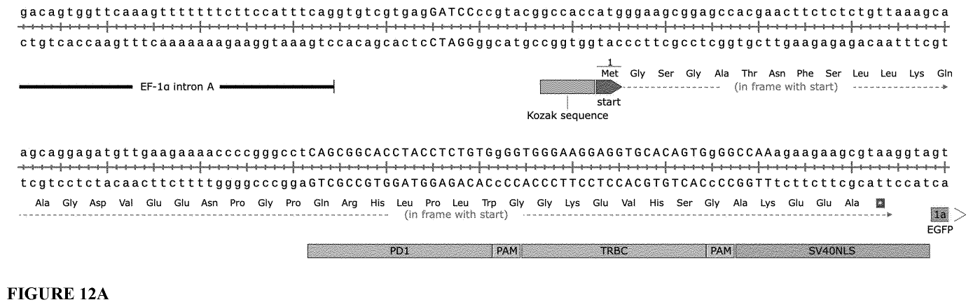

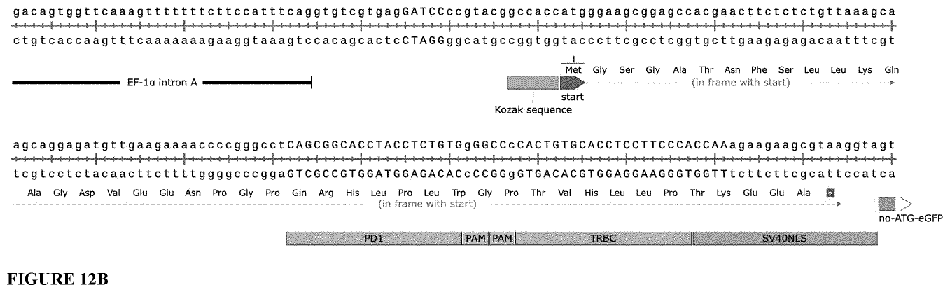

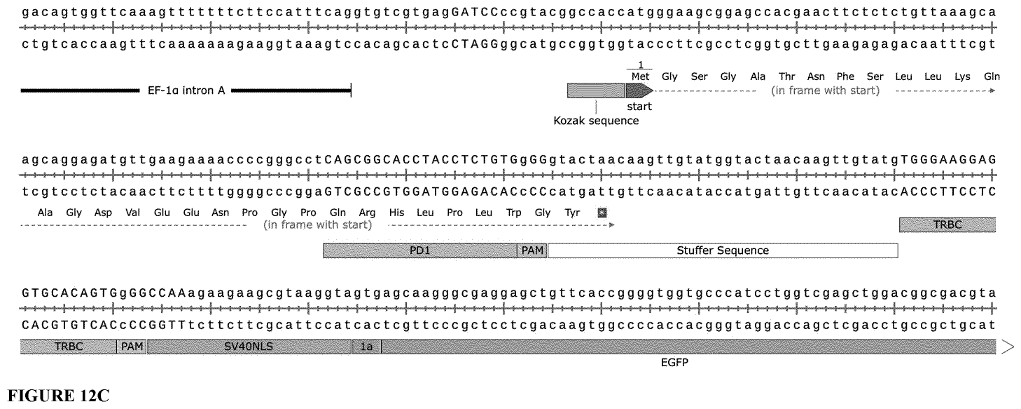

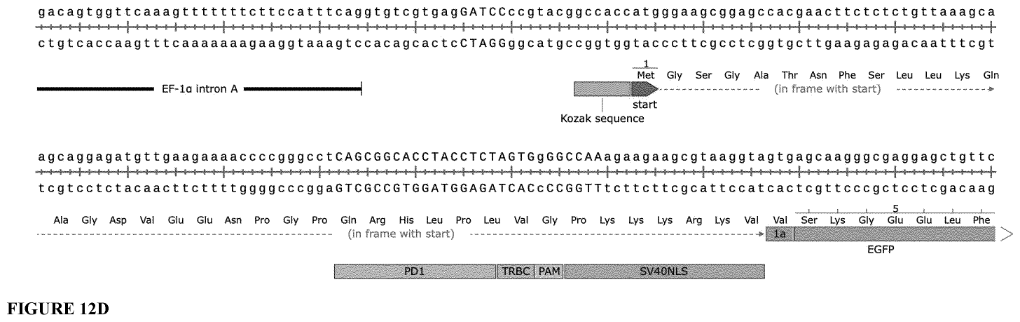

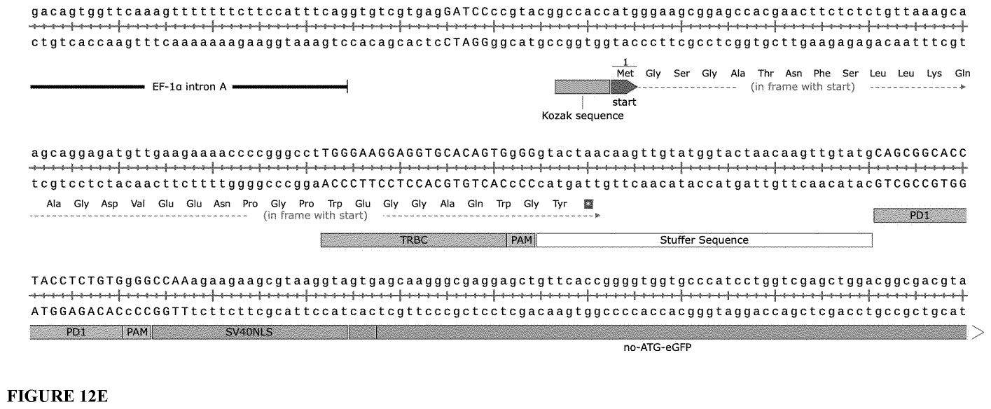

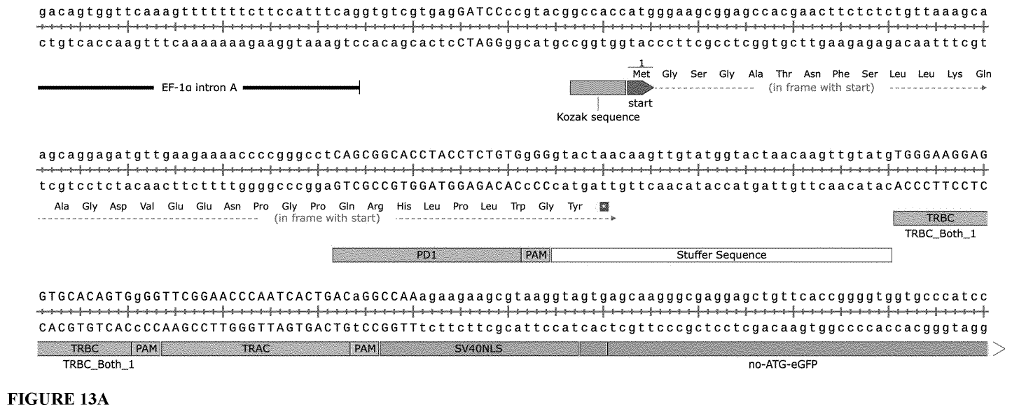

The first conformation was designed so that the two guide binding sequences and PAM complements were located directly in series on the reverse (3′ to 5′) strand of the molecular switch cassette (FIG. 12A). In this conformation, the second guide binding sequence and PAM complement were located immediately 5′ on the reverse (3′ to 5′) strand to the first guide binding sequence and PAM complement. This conformation is referred to herein as ‘TT’.

The second conformation was designed so that the first guide binding sequence and PAM complement were located on the reverse (3′ to 5′) strand and the second guide binding sequence and PAM complement were located on the forward (5′ to 3′) strand (FIG. 12B). In this conformation, the second guide binding sequence and PAM complement are located immediately 3′ on the opposing strand to the first guide binding sequence and PAM complement. This conformation is referred to herein as ‘TB’.

The third conformation was designed so that the two guide binding sequences and PAM complements were located on the reverse (3′ to 5′) strand but the guide binding sequences were separated by a 36 nucleotide ‘stuffer’ sequence (SEQ ID NO: 40, FIG. 12C). This conformation is referred to herein as ‘TstuffT’ The result of a deletion event between the first and second guide binding sequences is shown in FIG. 12D.

The fourth conformation was designed using the same layout as the third conformation (TstuffT), but with switching of the first and second guide binding sequences to see if the order of sequences per se had any impact (FIG. 12E). This confirmation is referred to herein as ‘Switch TstuffT’.

TABLE 6
Multiplex designs with two guides.

| Design | | Guide 1 | | Guide 2 | Stuffer |
|---|---|---|---|---|---|
| Name | Guide 1 | Strand | Guide 2 | Strand | Sequence |
| TT | PD1-1 | Reverse | TRBC- | Reverse | No |
| | | | Both-1 | | |
| TB | PD1-1 | Reverse | TRBC- | Forward | No |
| | | | Both-1 | | |
| TstuffT | PD1-1 | Reverse | TRBC- | Reverse | Yes |
| | | | Both-1 | | |
| Switch | TRBC- | Reverse | PD1-1 | Reverse | Yes |
| TstuffT | Both-1 | | | | |

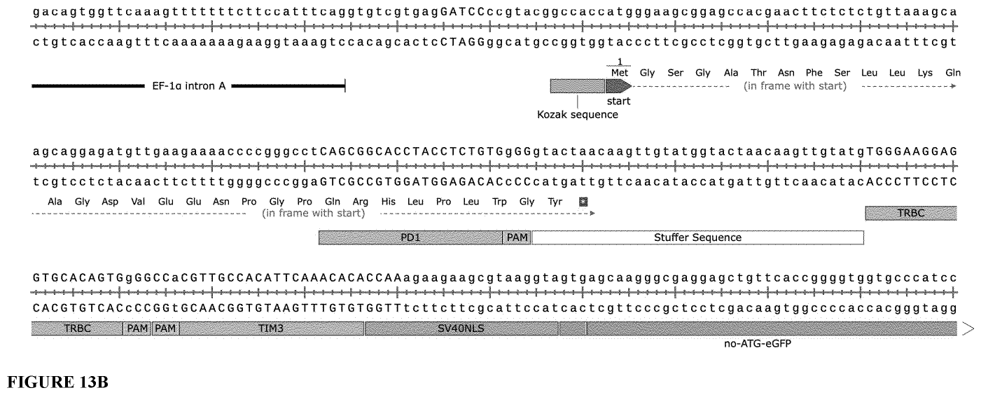

When using three (or a number divisible by three) guide binding sequences where the nucleic acid guide is predicted to result in a one base pair insertion, successful editing at all three locations would result in the insertion of three base pairs overall which would not result in a frameshift mutation. Therefore, the gene of interest could not be shifted back into frame. Therefore, a conformation was designed using guide binding sequences complementary to nucleic acid guides #1 (SEQ ID NO: 2), #3 (SEQ ID NO: 23), #5 (SEQ ID NO: 25) against PD-1 and TRBC, respectively (i.e., guide binding sequences with SEQ ID NOs: 13, 15 and 27). The first two guide binding sequences were separated by a 36-nucleotide stuff sequence comprising a stop codon (SEQ ID NO: 40) as performed above, but with a third guide binding sequence placed downstream of the second guide binding sequence on either the reverse (5′ to 3′) (referred to herein as ‘TTT’, FIG. 13A)) or forward (3′ tp 5′; ‘TTB’, FIG. 13B)) strand.

Editing and a frameshift at the first guide binding sequence is designed to remove the stop codon from the reading frame. Editing at the second and third guide binding sequences then works like the two guide multiplex designs, where two frameshifting edits will shift the gene of interest back into frame with a start codon and result in expression of the gene of interest. Successful editing may instead be a deletion event between the first two guide binding sequences and a frameshift 1 nucleotide insertion in the third guide binding sequence.

Multiplex conformations using three guides (also referred to as ‘triplex conformations’) are summarised in Table 7 and are depicted in FIGS. 13A and 13B.

TABLE 7
Multiplex designs with three guides.

| Design | | Guide 1 | | Guide 2 | | Guide 3 |
|---|---|---|---|---|---|---|
| Name | Guide 1 | Strand | Guide 2 | Strand | Guide 3 | Strand |
| TTT | PD1-1 | Reverse | TRBC- | Reverse | TRAC-1 | Reverse |
| | | | Both-1 | | | |
| TTB | PD1-1 | Reverse | TRBC- | Reverse | Tim3-42 | Forward |
| | | | Both-1 | | | |

### Lentiviral Production and Transfection of Cells

Lentiviruses were produced as described in Example 2 and electroporated into HEK293T cells as described in Example 2.

Cas9 and guide RNAs (i.e., as single guide RNA for each target) were precomplexed together as described in Example 2, except where three guide RNAs were used (i.e., for the TTT and TTB molecular cassettes) where 9 μg Cas9 and 60 pmol of each guide RNA was precomplexed. Where two guide RNAs were used 10 μg Cas9 and 100 pmol of each guide RNA was precomplexed. Electroporation was performed as described in Example 2.

### Analysis of Editing Events

Editing at the molecular switch site was evaluated through two different methods. The first method was to analyse editing at the guide binding site of the molecular switch cassette using Synthego's Inference of CRISPR Edits webtool as described in Example 2. However, this program is imperfect and the quality scores of the analysis can be low for multiplexing samples. Therefore, we also used a second method of PCR amplification of the molecular switch site using PCR (as described in Example 2), sanger sequencing the PCR amplicon (as described in Example 2) and aligned the resulting Sanger sequence to the plasmid map to see if changes have occurred. The alignment was performed using the programme SnapGene.

For the ‘TTB’ conformation using a guide binding sequence (SEQ ID NO: 42) complementary to a nucleic acid guide (SEQ ID NO: 41) targeting Tim3-42, the primers outlined in Table 8 were used. FACs was also performed as described in Example 2.

TABLE 8
Primers for targeting TIM3-42

| Gene | Forward Primer | Reverse Primer | Tm* | Band Size |
|---|---|---|---|---|
| TIM3-42 | AGCGAATCATCCTCCAAACAG | TGGGGCCTGTTAAACTTTAGGT | 72 | 522 |
| | (SEQ ID NO: 43) | (SEQ ID NO: 44) | | |

### Results

The results for the duplex designs are shown in Table 9. Table 9 reports genetic editing events at the guide binding sequence (′GBS*′) and endogenous gene locations (0). It is important to note that although this was a duplex design, a nucleic acid guide #5 (SEQ ID NO: 23) can target both TRBC1 and TRBC2 endogenous genes. As can be seen from Table 9, all of the molecular switch cassette designs resulted in co-editing at the guide binding sequence in the landing pad as well as the endogenous loci, along with successful expression of the gene of interest.

It was found that, when there was a stuffer sequence between the guide binding sequence (i.e., TstuffT and Switch TstuffT), deletion events between the two guide binding sites was more successful than two individual+1 bp insertion events. However, this still resulted in switching on of the expression of the gene of interest.

TABLE 9
Results from duplex design

| | | Editing event and percentage of | | | |
|---|---|---|---|---|---|
| | | detected alleles with event | | | |
| Design | % GFP | GBS* | PD1⋄ | TRBC1⋄ | TRBC2⋄ |
| TT | 58 | +1 bp ~60% | +1 bp | +1 bp | +1 bp |
| | | at each GBS | 99% | 99% | 99% |
| | | 22 bp | | | |
| | | deletion ~40% | | | |
| TB | 43 | +1 bp ~80% | +1 bp | +1 bp | +1 bp |
| | | at each GBS | 99% | 99% | 99% |
| | | 13 bp | | | |
| | | deletion ~20% | | | |
| TstuffT | 88 | 58 bp | +1 bp | +1 bp | +1 bp |
| | | deletion ~100% | 99% | 99% | 99% |
| Switch | 60 | 58 bp | +1 bp | +1 bp | +1 bp |
| TstuffT | | deletion ~100% | 99% | 99% | 99% |

*GBS = guide binding site in the molecular switch cassette. ⋄ = endogenous gene targets.
+1 indicates the detected mutation.

Table 10 shows the results for triplex switch designs. Table 10 shows that triplex designs also resulted in co-editing at the guide binding sites and the endogenous loci. However, the landing pad editing was more challenging to interpret as multiple events occurred. The TTT construct repeatably generated a 58-nucleotide deletion over the first two guides, and a 1 nucleotide insertion at the third site in the landing pad. The TTB construct appeared to give a mixture of different outcomes in the landing pad with a deletion across all three guides as a main outcome. There was also evidence of larger rearrangements. In addition to a 1 nucleotide insertion, the third nucleic acid guide produced a larger deletion (8 base pairs) at the endogenous locus, which may have contributed to the larger deletion at the landing pad site.

TABLE 10
Results from triplex design

| | | Editing event and percentage of detected alleles with event | | | | |
|---|---|---|---|---|---|---|
| Design | % GFP | PD1⋄ | TRBC1*⋄ | TRBC2*⋄ | Tim3⋄ | TRAC⋄ |
| TTT | 32% | +1 bp 99% | +1 bp 99% | +1 bp 99% | N.A. | +1 bp 87% |
| | | | | | | −4 bp 6% |
| TTB | 45% | +1 bp 99% | +1 bp 99% | +1 bp 99% | +1 bp 83% | N.A |
| | | | | | −8 bp 10% | |

*targeted using the same nucleic acid guide sequence.
⋄endogenous gene targets.

1. An expression vector comprising:
(a) a coding nucleic acid sequence encoding a polypeptide of interest, wherein the coding nucleic acid sequence is out of frame with a start codon such that the polypeptide of interest is not constitutively expressed from the expression vector; and
(b) a guide-binding sequence located upstream of the coding nucleic acid sequence, wherein the guide-binding sequence comprises a sequence complementary to a nucleic acid guide, and wherein the guide-binding sequence is present in one or more endogenous genes,
wherein binding of a nucleic acid guide to the guide-binding sequence directs a Cas enzyme to produce a frameshift mutation in a nucleic acid sequence of the expression vector resulting in expression of the polypeptide of interest; and
wherein binding of a nucleic acid guide to the guide-binding sequence in an endogenous gene directs a Cas enzyme to disrupt expression of the endogenous gene.

2-11. (canceled)

12. The expression vector of claim 1, wherein the endogenous gene encodes an immune checkpoint molecule; and/or wherein the endogenous gene is selected from: TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2; and/or wherein the coding sequence encodes a chimeric antigen receptor.

13-14. (canceled)

15. The expression vector of claim 1, wherein the guide-binding sequence or nucleic acid guide is 8 to 50 nucleic acid residues in length; and/or wherein the guide-binding sequence comprises a sequence selected from: SEQ ID NO: 13-15, 22, 27-30, and 42; and/or wherein the nucleic acid guide comprises or consists of a sequence selected from: SEQ ID NO: 2-3, 23-26, 36-37, and 41.

16-20. (canceled)

21. The expression vector of claim 1, wherein the Cas enzyme is a Cas9; and/or wherein the guide-binding sequence is located adjacent to a sequence complementary to a protospacer adjacent motif (PAM); and/or wherein the expression vector is an adenovirus, a retrovirus, an adeno-associated virus, or a lentivirus; and/or wherein the expression vector is an integrated expression vector.

22-30. (canceled)

31. A combination comprising:
(a) an expression vector comprising:
(i) a coding nucleic acid sequence encoding a polypeptide of interest, wherein the coding nucleic acid sequence is out of frame with a start codon such that the polypeptide of interest is not constitutively expressed from the expression vector; and
(ii) a guide-binding sequence located upstream of the coding nucleic acid sequence, wherein the guide-binding sequence comprises a sequence complementary to a nucleic acid guide, and wherein the guide-binding sequence is present in one or more endogenous genes; and
(b) a nucleic acid guide that is able to bind to the guide-binding sequence in the expression vector and directs a Cas enzyme to produce a frameshift mutation in a nucleic acid sequence of the expression vector resulting in expression of the polypeptide of interest, and wherein the nucleic acid guide is able to bind to the guide-binding sequence in the one or more endogenous genes and direct a Cas enzyme to disrupt expression of the one or more endogenous genes.

32. An isolated cell comprising an expression vector comprising:
(a) a coding nucleic acid sequence encoding a polypeptide of interest, wherein the coding nucleic acid sequence is out of frame with a start codon such that the polypeptide of interest is not constitutively expressed from the expression vector; and
(b) a guide-binding sequence located upstream of the coding sequence, wherein the guide-binding sequence comprises a sequence complementary to a nucleic acid guide, and wherein the guide-binding sequence is present in one or more endogenous genes,
wherein binding of a nucleic acid guide to the guide-binding sequence directs a Cas enzyme to produce a frameshift mutation in a nucleic acid sequence of the vector resulting in expression of the polypeptide of interest; and
wherein binding of a nucleic acid guide to the guide-binding sequence in an endogenous gene directs a Cas enzyme to disrupt expression of the endogenous gene.

33. The isolated cell of claim 32, further comprising a Cas enzyme and/or a nucleic acid guide that is able to bind to the guide-binding sequence in the expression vector and direct a Cas enzyme to produce a frameshift mutation in a nucleic acid sequence of the expression vector resulting in expression of the polypeptide of interest, and wherein the nucleic acid guide is able to bind to the guide-binding sequence in the one or more endogenous genes and direct a Cas enzyme to disrupt expression of the endogenous gene; and/or
wherein the cell is ex vivo; and/or wherein the cell is a blood cell, a stem cell, an immune cell, a dermal cell, or a T lymphocyte; and/or
wherein the expression vector is integrated into the cell genome.

34-55. (canceled)

56. A method of expressing a polypeptide of interest in a cell, and concurrently disrupting expression of one or more endogenous genes in the cell, said method comprising:
(i) providing a cell with an expression vector comprising:
(a) a coding nucleic acid sequence encoding a polypeptide of interest, wherein the coding nucleic acid sequence is out of frame with a start codon such that the polypeptide of interest is not constitutively expressed from the expression vector; and
(b) a guide-binding sequence located upstream of the coding sequence, wherein the guide-binding sequence comprises a sequence complementary to a nucleic acid guide, and wherein the guide-binding sequence is present in one or more endogenous genes;
(ii) providing the cell with a nucleic acid guide complementary to the guide-binding sequence of the vector and complementary to the guide-binding sequence in one or more endogenous genes; and
(iii) providing the cell with a Cas enzyme,
wherein binding of the nucleic acid guide to the guide-binding sequence in the expression vector directs the Cas enzyme to produce a frameshift mutation in a nucleic acid sequence of the expression vector resulting in expression of the polypeptide of interest; and
wherein binding of the nucleic acid guide to the guide-binding sequence in one or more endogenous genes directs the Cas enzyme to disrupt expression of the endogenous gene(s) in the cell.

57. The method of claim 56, wherein the one or more endogenous gene(s) encode an immune checkpoint molecule; and/or wherein the one or more endogenous genes is/are selected from: TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2.

58-59. (canceled)

60. A method of treating a cancer, autoimmune disorder, skin disease, inflammatory disease, ion channel disease, endocrine disease, extracellular matrix diseases, or metabolic disorder, said method comprising:
(i) providing a population of cells obtained from a donor subject;
(ii) introducing an expression vector comprising:
(a) a coding nucleic acid sequence encoding a polypeptide of interest, wherein the coding nucleic acid sequence is out of frame with a start codon such that the polypeptide of interest is not constitutively expressed from the expression vector; and
(b) a guide-binding sequence located upstream of the coding sequence, wherein the guide-binding sequence comprises a sequence complementary to a nucleic acid guide, and wherein the guide-binding sequence is present in one or more endogenous genes;
(iii) introducing a nucleic acid guide complementary to the guide-binding sequence of the vector and complementary to the guide-binding sequence in the one or more endogenous genes into the cells,
such that binding of the nucleic acid guide to the guide-binding sequence in the expression vector directs a Cas enzyme to produce a frameshift mutation in a nucleic acid sequence of the expression vector resulting in expression of the polypeptide of interest, and wherein binding of the nucleic acid guide to the guide-binding sequence in one or more endogenous genes directs a Cas enzyme to disrupt expression of the endogenous genes; and
(v) administering an effective amount of the cells to a recipient subject in need of treatment.

61-62. (canceled)

63. The method of according to claim 60, wherein the Cas enzyme is a Cas9; and/or wherein (iii) further comprises introducing a Cas enzyme into the cells; and/or wherein the expression vector is integrated into the genomes of the cells.

64. The method of claim 60, wherein the donor subject is the same as the recipient subject; and/or wherein the cells are blood cells, stem cells, immune cells, dermal cells, or lymphocytes.

65-71. (canceled)

72. The method of claim 60, wherein the nucleic acid guide or guide-binding sequence is 8 to 50 nucleic acid residues in length; and/or wherein the nucleic acid guide comprises or consists of a sequence selected from: SEQ ID NO: 2-3, 23-26, 36-37 and 41; and/or wherein the guide-binding sequence comprises a sequence selected from: SEQ ID NO: 13-15, 22, 27-30 and 42; and/or wherein the one or more endogenous gene(s) encode an immune checkpoint molecule; and/or wherein the one or more endogenous genes is/are selected from: TRAC, PD-1, CD38, CD39, TIM3, TIGIT, LAG3, TRBC1, TRBC2, CISH, CD70, B2M, HLA-A, HLA-B, HLA-C, HLA-E, HLA-G, NKG2A, NKG2D, CBLB, TGFBR1, and TGFBR2.

73-79. (canceled)

80. The method according to claim 60, wherein the cancer is selected from: mesothelioma; lung cancer pancreatic cancer; oesophageal adenocarcinoma, ovarian cancer, breast cancer, colorectal cancer, bladder cancer, haematological cancer, leukaemia or lymphoma, chronic lymphocytic leukaemia (CLL), mantle cell lymphoma (MCL), multiple myeloma, acute lymphoid leukaemia (ALL), Hodgkin lymphoma, B-cell acute lymphoid leukaemia (BALL), T-cell acute lymphoid leukaemia (TALL), small lymphocytic leukaemia (SLL), B cell prolymphocytic leukaemia, blastic plasmacytoid dendritic cell neoplasm, Burkitt's lymphoma, diffuse large B cell lymphoma (DLBCL), DLBCL associated with chronic inflammation, chronic myeloid leukaemia, myeloproliferative neoplasms, follicular lymphoma, paediatric follicular lymphoma, hairy cell leukaemia, small cell- or a large cell-follicular lymphoma, malignant lymphoproliferative conditions, MALT lymphoma (extranodal marginal zone lymphoma of mucosa associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukaemia, splenic diffuse red pulp small B-cell lymphoma, hairy cell leukaemia-variant, lymphoplasmacytic lymphoma, a heavy chain disease, plasma cell myeloma, solitary plasmacytoma of bone, extraosseous plasmacytoma, nodal marginal zone lymphoma, paediatric nodal marginal zone lymphoma, primary cutaneous follicle centre lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukaemia (AML), and unclassifiable lymphoma.

81. The method of claim 60, wherein the autoimmune disorder is selected from: rheumatoid arthritis, psoriasis, arthritis, type 1 diabetes mellitus, and lupus.

82. The method of claim 60, wherein the metabolic disorder is selected from: Malnutrition-inflammation atherosclerosis syndrome, Gaucher disease, mucopolysaccharidosis type II (also known as Hunter syndrome), Krabbe's Leukodystrophy (also known as Krabbe's disease), stroke, and type 2 diabetes mellitus.

83. The method of claim 60, wherein the inflammatory disease is selected from: Alzheimer's disease, Parkinson's disease, fatty liver disease, endometriosis, type 2 diabetes mellitus, type 1 diabetes mellitus, inflammatory bowel disease, asthma, rheumatoid arthritis, ankylosing spondylitis, antiphospholipid antibody syndrome, gout, myositis, scleroderma, Sjogren's syndrome, systemic lupus erythematosus, and vasculitis.

84. The method of claim 60, wherein the skin disease is selected from: psoriasis, hives, vitiligo, and ichthyosis.

85-89. (canceled)

90. The method according to claim 56, wherein the Cas enzyme is a Cas9; and/or wherein the cell is ex vivo; and/or wherein the cell is a blood cell, a stem cell, an immune cell, a dermal cell, or a T lymphocyte.

91. The method according to claim 56, wherein the nucleic acid guide or guide binding sequence is 8 to 50 nucleic acid residues in length; and/or wherein the nucleic acid guide (ii) comprises or consists of a sequence selected from: SEQ ID NO: 2-3, 23-26, 36-37, and 41; and/or wherein the guide-binding sequence comprises a sequence selected from: SEQ ID NO: 13-15, 22, 27-30, and 42.
